(19) United States
(12) Patent Application Publication
(10) Pub. No.: US 2024/0011055 A1
(43) Pub. Date: Jan. 11, 2024

(54) PRECISE GENOME DELETION AND REPLACEMENT METHOD BASED ON PRIME EDITING

(71) Applicant: University of Washington, Seattle, WA (US)

(72) Inventors: Jay Ashok Shendure, Seattle, WA (US); Wei Chen, Seattle, WA (US); Junhong Choi, Seattle, WA (US)

(73) Assignee: University of Washington, Seattle, WA (US)

(21) Appl. No.: 18/251,514

(22) PCT Filed: Nov. 4, 2021
(86) PCT No.: PCT/US2021/058079

§ 371 Date: May 2, 2023

Related U.S. Application Data
(60) Provisional application No. 63/110,304, filed on Nov. 5, 2020.

(51) Int. Cl. C12N 15/90; C12N 15/11; C12N 9/22; C12N 9/12

(52) U.S. Cl. CPC C12N 15/907; C12N 15/11; C12N 9/22; C12N 9/1241; C12N 2310/20

(57) ABSTRACT

Disclosed are methods and related compositions for genomic editing. In one aspect, methods of editing double stranded DNA (dsDNA) use first and second editing complexes specific for first and second target sequences on the sense and antisense strands of the dsDNA molecule, respectively. Each editing complex comprises an extended guide RNA associated with a fusion editor protein, which comprises a functional nickase domain and a functional reverse transcriptase domain. The respective guide RNAs guide their associated fusion editor proteins to the dsDNA, which implement single stranded breaks on opposite strands of the dsDNA. The respective reverse transcriptase domains generate 3′ overhangs. Repair of the dsDNA excises the portion of dsDNA disposed between the two single-stranded breaks. A variety of configurations and applications of the method are disclosed, providing flexible, facile, efficient, and precise methods to impose genetic manipulations.

### CROSS-REFERENCE TO RELATED APPLICATION

This application claims the benefit of Provisional Application No. 63/110,304, filed Nov. 5, 2020, the disclosure of which is incorporated herein by reference in its entirety.

### STATEMENT OF GOVERNMENT LICENSE RIGHTS

This invention was made with Government support under Grant No. UM1 HG009408, awarded by the National Institutes of Health. The Government has certain rights in the invention.

### STATEMENT REGARDING SEQUENCE LISTING

The sequence listing associated with this application is provided in text format in lieu of a paper copy and is hereby incorporated by reference into the specification. The name of the text file containing the sequence listing is 3915-P1162WOUW_Seq_List_FINAL_20211101_ST25.txt. The text file is 28 KB; was created on Nov. 1, 2021; and is being submitted via EFS-Web with the filing of the specification.

### BACKGROUND

The ability to precisely manipulate the genome can enable investigations of the function of specific genomic sequences, including genes and regulatory elements. Within the past decade, CRISPR-Cas9-based technologies have proven transformative in this regard, allowing precise targeting of a genomic locus, with a quickly expanding repertoire of editing or perturbation modalities. Among these, the precise and unrestricted deletion of specific genomic sequences is particularly important, with use cases in both functional genomics and gene therapy.

Currently, the leading method for programming genomic deletions uses a pair of CRISPR single-guide RNAs (sgRNAs) that each target a protospacer-adjacent motif (PAM) sequence, generating a pair of nearby DNA double-strand breaks (DSBs). Upon simultaneous cutting of two sites, cellular DNA damage repair factors often ligate two ends of the genome without the intervening sequence through non-homologous end joining (NHEJ) (FIG. 1A). Although powerful, this approach has several limitations: 1) An attempt to induce a deletion, particularly a longer deletion, often results in short insertions or deletions (indels; typically less than 10-bp) near one or both DSBs, with or without the intended deletion; 2) Other unintended mutations including large deletions and more complex rearrangements can frequently occur, and go undetected for technical reasons; 3) DSBs are a cytotoxic insult; and 4) The junctions of genomic deletions programmed by this method are limited by the distribution of naturally occurring PAM sites. Notwithstanding these limitations, various studies have employed this strategy to great effect, e.g. to investigate the function of genes and regulatory elements, as well as towards gene therapy. However, limited precision, DSB toxicity and the inability to program arbitrary deletions have handicapped the utility of CRISPR-Cas9-induced deletions in functional and therapeutic genomics.

Recently “prime editing” has been described, which expands the CRISPR-Cas9 genome editing toolkit in various wayshttps://paperpile.com/c/gGxRnW/t6eb1. Prime editing utilizes a Prime Editor-2 enzyme, which is a Cas9 nickase (Cas9 H840A) fused with a reverse-transcriptase, and a 3′-extended sgRNA (prime-editing sgRNA or pegRNA). The Prime Editor-2 enzyme and pegRNA complex can nick one strand of the genome and attach a 3′ single-stranded DNA flap to the nicked site following the template RNA sequence in the pegRNA molecule. By including homologous sequences to the neighboring region, DNA damage repair factors can incorporate the 3′-flap sequence into the genome. The incorporation rate can be further enhanced using an additional sgRNA, which makes a nick on the opposite strand, boosting DNA repair with the 3′-flap sequence but often with a decrease in precision (strategy referred to as PE3/PE3b) (FIG. 1B). An advantage of prime editing lies with its encoding of both the site to be targeted and the nature of the repair within a single molecule, the pegRNA. The PE3 strategy has been used to show that a single pegRNA/sgRNA pair could be used to program deletions ranging from 5 to 80 bp achieving high efficiency (52-78%) with modest precision (on average, 11% rate of unintended indels). However, even the PE3 strategy faces major difficulties in programming deletions larger than 100 bp. Moreover, observed efficiencies fall precipitously for deletions larger than 20 bp.

Accordingly, despite the advances in the art of genomic editing, a need remains for facile, efficient, and precise methods to impose genetic manipulations (e.g., deletions and insertions). The present disclosure addresses these and related needs.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the disclosure provides a method of editing a double stranded DNA (dsDNA) molecule with a sense strand and antisense strand. The method comprises contacting the dsDNA molecule with a first editing complex specific for a first target sequence on the sense strand of the dsDNA molecule and a second editing complex specific for a second target sequence on the antisense strand of the dsDNA molecule. The first editing complex and the second editing complex each comprise a fusion editor protein and an extended guide RNA molecule associated therewith. The fusion editors each comprise a functional nickase domain and a functional reverse transcriptase domain. The extended guide RNA molecule of the first editing complex comprises a first guide domain with a first sequence that hybridizes to the first target sequence and a first extended domain at the 3′ end. The extended guide RNA molecule of the second editing complex comprises a second guide domain with a second sequence that hybridizes to the second target sequence and a second extended domain at the 3′ end. The method further comprises permitting the functional nickase domain of the first editing complex and the functional nickase domain of the second editing complex to create a first single-stranded break and a second single-stranded break in opposite strands of the dsDNA molecule at the first target sequence and second target sequence, respectively. Next, the method comprises permitting the functional reverse transcriptase domain of the first editing complex to generate a first 3′ overhang from the first single-stranded break using the first extended domain as template, and permitting the functional reverse transcriptase domain of the second editing complex to generate a second 3′ overhang from the second single-stranded break using the second extended domain as template. Finally, the method comprises repairing the dsDNA molecule by excising the portion of the dsDNA originally disposed between the first single-stranded break and second single stranded break and incorporating the first 3′ overhang and second 3′ overhang into the repaired dsDNA molecule.

In some embodiments, the functional nickase domain of the first editing complex and the functional nickase domain of the second editing complex are independently CRISPR-associated (Cas) enzyme, Pyrococcus furiosus Argonaute, and the like, or a functional nickase domain derived therefrom. In some embodiments, the Cas is Cas9, Cas12, Cas13, Cas3, CasED, and the like. In some embodiments, the functional reverse transcriptase domain of the first editing complex and the functional reverse transcriptase domain of the second editing complex are independently M-MLV RT, HIV RT, group II intron RT (TGIRT), superscript IV, and the like, or a functional domain thereof.

In some embodiments, the first target sequence is disposed in a more 5′ location in the sense strand than the reverse complement of the second target sequence. In some embodiments, the first target sequence is disposed in a more 3′ location in the sense strand than the reverse complement of the second target sequence. In some embodiments, the first 3′ overhang and the second 3′ overhang are reverse complements of each other and hybridize in the repairing step.

In some embodiments, the first 3′ overhang comprises a first repair domain with a sequence that corresponds to a sequence immediately 5′ to the second 3′ overhang in the antisense strand, and wherein the second 3′ overhang comprises a second repair domain with a sequence that corresponds to sequence immediately 5′ to the first 3′ overhang in the sense strand. In some embodiments, the first 3′ overhang further comprises an insertion sequence 5′ to the first repair domain, and wherein the second 3′ overhang comprises a reverse complement sequence of the insertion sequence 5′ to the second repair domain.

In some embodiments, the first 3′ overhang comprises a first repair domain with a sequence that corresponds to a sequence immediately 3′ to the second single stranded break, and wherein the second 3′ overhang comprises a second repair domain with a sequence that corresponds to a sequence immediately 3′ to the first single stranded break, whereby the repairing step results in an inversion of the sequence corresponding to the portion of the dsDNA originally disposed between the first single-stranded break and second single stranded break.

In some embodiments, the first 3′ overhang comprises a first repair domain with a sequence that corresponds to a first end domain of an insertion DNA fragment, wherein the second 3′ overhang comprises a second repair domain with a sequence that corresponds to a second end domain of the insertion DNA fragment, and wherein the first end domain and second end domain are at opposite ends of the insertion DNA fragment or are at distinct sites within a larger dsDNA molecule.

In some embodiments, the portion of the dsDNA molecule originally disposed between the first single-stranded break and second single stranded break that is excised is at least 5 nucleotides long. In some embodiments, the portion of the dsDNA molecule originally disposed between the first single-stranded break and second single stranded break that is excised is between about 10 nucleotides and 1,000,000 nucleotides long.

In some embodiments, the first editing complex and/or the second editing complex comprise(s) an additional functional domain configured to enhance the efficiency of 3′-overhang generation. In some embodiments, the fusion editor protein of the first editing complex and/or the second editing complex comprise(s) an additional functional domain configured to enhance the efficiency of DNA repair using generated 3′ overhangs.

In some embodiments, the first guide domain and second guide domain are independently between about 20 and about 200 nucleotides long. In some embodiments, the first guide domain and second guide domain are independently between about 25 and 100 nucleotides long, between about 25 and 50 nucleotides long, or between about 25 and nucleotides long.

In some embodiments, the first guide domain and the second guide domain are configured to be compatible with the first editing complex and the second editing complex, respectively, and/or one or more nucleotide residues in the first guide domain and/or the second guide domain are modified with 2′-O-methylation, locked nucleic acids, peptide nucleic acids, or a similar functionally modified nucleic acid moiety.

In some embodiments, the e first extended domain and the second extended domain are independently at least about 10 nucleotides long. In some embodiments, the first extended domain and the second extended domain are independently about 10 nucleotides to about 40 nucleotides long.

In some embodiments, the method is performed in a cell in vitro. In some embodiments, the method is performed in a cell in vivo. In some embodiments, the method is a therapeutic method comprising deletion of a genomic sequence, inverting a genomic sequence, interchromosomal rearrangement, and/or inserting a new sequence into a target region or site of the genome.

In some embodiments, the method is expanded to encompass multiple pairs of first and second editing complexes to implement edits at multiple locations in the dsDNA molecule. The method can comprise contacting the dsDNA with multiple pairs of first and second editing complexes, wherein each pair of first and second editing complexes targets different pairs of first and second target sequences within the dsDNA.

In some embodiments, the method comprises pooling a plurality of pegRNAs or a plurality of nucleic acid molecules encoding the pegRNAs, and contacting a cell comprising the dsDNA molecule with the pool of the plurality of pegRNAs or a plurality of nucleic acid molecules encoding the pegRNAs. In some embodiments, the method also comprises contacting the cell with one or more fusion editor proteins or one or more nucleic acid molecules encoding the one or more fusion editor proteins, and permitting the fusion editor proteins to express and/or complex within the cell.

In another aspect, the disclosure provides a method of editing one or more double stranded DNA (dsDNA) molecules in a cell. The method comprises contacting the cell with one or more pairs of first and second editing complexes, or one or more nucleic acids encoding components of the one or more pairs of first and second complexes and permitting the components to be expressed and assembled in the cell. For each pair of the one or more pairs first and second editing complexes, the following applies:
the first editing complex is specific for a first target sequence on the sense strand of the dsDNA molecule and the second editing complex specific for a second target sequence on the antisense strand of the dsDNA molecule;
the first editing complex and the second editing complex each comprise a fusion editor protein and an extended guide RNA molecule associated therewith, wherein the fusion editors each comprise a functional nickase domain and a functional reverse transcriptase domain;
the extended guide RNA molecule of the first editing complex comprises a first guide domain with a first sequence that hybridizes to the first target sequence and a first extended domain at the 3′ end; and
the extended guide RNA molecule of the second editing complex comprises a second guide domain with a second sequence that hybridizes to the second target sequence and a second extended domain at the 3′ end.

The method comprises (for each pair of first and second editing complexes) permitting the functional nickase domain of the first editing complex and the functional nickase domain of the second editing complex to create a first single-stranded break and a second single-stranded break in opposite strands of the dsDNA molecule at the first target sequence and second target sequence, respectively; permitting the functional reverse transcriptase domain of the first editing complex to generate a first 3′ overhang from the first single-stranded break using the first extended domain as template, and permitting the functional reverse transcriptase domain of the second editing complex to generate a second 3′ overhang from the second single-stranded break using the second extended domain as template; and repairing the dsDNA molecule by excising the portion of the dsDNA originally disposed between the first single-stranded break and second single stranded break and incorporating the first 3′ overhang and second 3′ overhang into the repaired dsDNA molecule.

In some embodiments, the method comprises contacting the cell with a plurality of pairs of first and second editing complexes, or a plurality of nucleic acids encoding components of the plurality of pairs of first and second complexes and permitting the components to be expressed and assembled in the cell. Each pair of first and second editing complexes targets different first and second target sequences on the one or more dsDNA molecules in the cell.

In another aspect, the disclosure provides a kit comprising a first editing complex and the second editing complex as described herein, wherein the first target sequence on the sense strand and second target sequence on the antisense strand are separated by an intervening sequence. The first editing complex and the second editing complex are configured to delete intervening sequence, to invert the intervening sequence, and/or inserting one or more new sequences at the first and/or second single stranded breaks induced by the first editing complex and the second editing complex in the target dsDNA molecule.

### DESCRIPTION OF THE DRAWINGS

The foregoing aspects and many of the attendant advantages of this disclosure will become more readily appreciated as the same become better understood by reference to the following detailed description, when taken in conjunction with the accompanying drawings, wherein:

FIGS. 1A-1H. Precise episomal deletions using PRIME-Del. (1A-1C) Schematic of Cas9/paired-sgRNA deletion strategy (1A), PE3 (1B), and PRIME-Del (1C). For PRIME-Del in 1C, a pair of pegRNAs encodes the sites to be nicked at each end of the intended deletion but on opposing strands, as well as 3′ flaps. In the illustrated embodiment, the 3′ flaps contain sequence that is complementary to the region targeted by the other pegRNA. Letter designations are imposed indicting how the flaps hybridize with the targeted dsDNA sequence and are integrated into the repaired, edited sequence. (1D) Cartoon representation of deletions programmed within the episomally-encoded eGFP gene (not drawn to a scale). (1E) PRIME-Del-mediated deletion efficiencies and error frequencies (with or without intended deletion) were measured for 24-bp, 91-bp, and 546-bp deletion experiments in HEK293T cells (mean over n=5 transfection replicates). Sequencing reads were classified as without indel modifications (“No editing”), indel errors without the intended deletion, indel errors with the intended deletion, and correct deletion without error. (1F) PRIME-Del-mediated deletion efficiency was measured for the 546-bp deletion experiment using three methods. (mean±SD over n=3 transfection replicates) (1G) Insertion, deletion and substitution error frequencies across sequencing reads from 546-bp deletion experiment. Reads were aligned to reference sequence either without (top) or with (bottom) deletion. Plots are from single-end reads with collapsing of UMIs to reduce sequencing errors; also shown with additional replicates and error-class-specific scales in FIG. 6E. Note that only one of the two 3′-DNA-flaps is covered by the sequencing read in amplicons lacking the deletion (labeled as ‘wild-type’). (1H) Insertion, deletion and substitution error frequencies across the amplicons from 546-bp deletion experiment after merging paired-end sequencing reads.

FIGS. 2A-2F. Concurrent programming of deletion and insertion using PRIME-Del. (2A) Schematic of strategy PRIME-Del variation configured to insert a sequence between the break sites. The encoded 3′ flaps contain sequence that is complementary to the region targeted by the other pegRNA, as in FIG. 1C, but also contain additional sequence to be inserted. The additional sequence is presented in reverse complementary format corresponding to the pair of corresponding 3′ flaps such that they anneal during the repair step, resulting in inserted dsDNA sequence. The regions of correspondence are indicated with letter designations, specifically with the inserted sequence designated by B/b. (2B) Conventional strategy for deletion with Cas9 and pairs of sgRNAs. Potential deletion junctions are restricted by the natural distribution of PAM sites. (2C) Pairs of pegRNAs were designed to encode five insertions, ranging in size from 3 to 30 bp, together with a 546 bp deletion in eGFP. (2D) Estimated deletion efficiencies and indel error frequencies (with or without intended deletion) in using these pegRNA pairs to induce concurrent deletion and insertion in HEK293T cells. (mean over n=3 transfection replicates) (2E) Representative insertion, deletion and substitution error frequencies plotted across sequencing reads from concurrent 546-bp deletion and 30-bp insertion condition. Plots are from single-end reads without UMI correction. Note that only one of the two 3′-DNA-flaps is covered by the sequencing read in amplicons lacking the deletion (labeled as ‘wild-type’). (2F) The percentage of reads containing the programmed deletion that also contain the programmed insertion. (mean±SD over n >3 transfection replicates)

FIGS. 3A-3G. Precise genomic deletions using PRIME-Del. (3A) Schematic of generation of the eGFP-integrated HEK293T cell line. (3B) Estimated deletion efficiencies and error frequencies in using PRIME-Del for concurrent deletion and insertion on genomically integrated eGFP in HEK293T cells. (mean over n=3 transfection replicates) (3C) Representative insertion, deletion and substitution error frequencies plotted across sequencing reads from concurrent 546-bp deletion and 30-bp insertion condition on genome-integrated eGFP. Plots are from single-end reads without UMI correction. (3D) Cartoon representation of deletions programmed within the HPRT1 gene. (3E) Deletion efficiencies measured for the 118-bp and 252-bp deletion using either PRIME-Del or Cas9/paired-sgRNA (abbreviated to Cas9) strategies in HEK293T cells, quantified using either the unique-molecular identifier-based sequencing assay (UMI) or the droplet-digital PCR (ddPCR) assay. (mean±SD over n=3 transfection replicates). (3F) Representative insertion, deletion and substitution error frequencies plotted across sequencing reads from 118-bp deletion (left) and 252-bp deletion (right) at HPRT exon 1, using the Cas9/paired-sgRNA strategy. Different error classes are colored the same as in (3C). (3G) Same as (3F), but for PRIME-Del strategy.

FIGS. 4A-4E. Characterizing PRIME-Del across the genome. (4A) Estimated deletion efficiencies and indel error frequencies for different deletions across the genome for PRIME-Del (left) and Cas9/paired-sgRNA (right) methods. (mean over n=3 transfection replicates) UMI-based sequencing assay was used for quantification (except the GC-rich amplicon of FMR1*, where added DMSO interfered with the UMI-addition reaction). (4B) Schematic of a sequence inversion event, which is a known error mode in Cas9/paired-sgRNA-mediated deletion. (4C) Estimated inversion frequencies for different deletions across the genome for PRIME-Del (left) and Cas9/paired-gRNA (right) methods. (mean over n=3 transfection replicates) Note that whereas they are observed for all but one of the Cas9/paired-sgRNA-mediated deletions at an appreciable frequency, virtually no inversions are observed for any of these ten deletions using PRIME-Del. (4D) Deletion efficiencies measured for 1-kb and 10-kb deletions at HPRT1 using either PRIME-Del (left) or Cas9/paired-sgRNA (right) with ddPCR-based assay in HEK293T cells. (mean±SD over n=3 transfection replicates). (4E) Fraction of reads with precise deletion measured for the 1-kb and 10-kb deletion on HPRT1 gene with either PRIME-Del (left) or Cas9/paired-sgRNA (right) using sequencing of the deletion amplicons. (mean±SD over n=3 transfection replicates).

FIG. 5. Potential advantages of using PRIME-Del in various genome editing applications. The PRIME-Del strategy can be used to program precise genomic deletions without generation of short indel errors at Cas9 target sequences. Precision deletion, combined with ability to insert a short arbitrary sequence at the deletion junction, may allow robust gene knockout of active protein domains without generating a premature in-frame stop codon, which can trigger the nonsense-mediated decay (NMD) pathway. PRIME-Del may also allow replacement of genomic regions up to 10 kb with arbitrary sequences such as epitope tags or RNA transcription start sites. Single-stranded breaks generated during PRIME-Del are likely to be less toxic to the cell when multiple regions are edited in parallel potentially facilitating its multiplexing.

FIGS. 6A-6E. Error profiles with PRIME-Del deletions targeting episomally encoded eGFP. (6A) Sample preparation schematic for amplicon sequencing. Region around the segment targeted for deletion is amplified from the genomic DNA using two-step PCR amplification that appends sequencing adaptors in the second step. (6B-6D) Insertion, deletion and substitution error frequencies across sequencing reads for 24-bp deletion (6B), 91-bp deletion (6C), and 546-bp deletion (6D). These are based on single-end sequencing, with five replicates per experiment, all sequenced on one run, overlaid. Note that except for 24-bp deletion, only one of the two 3′-DNA-flaps is covered by the sequencing read in amplicons lacking the deletion (labeled as ‘wild-type’). Y-axis scaling is different for each plot. (6E) Error frequencies across 546-bp deletion after repeating amplification to allow unique molecular identifier (UMI) correction. PCR duplicates identified by UMIs were collapsed into a single read by taking the most frequent sequence sharing the same UMI. These are based on single-end sequencing, with three replicates per experiment, all sequenced on one run, overlaid. Y-axis scaling is different for each plot.

FIGS. 7A-7C. Error profiles with concurrent deletion and insertion at episomally or genornically encoded eGFP. (7A) Insertion, deletion and substitution error frequencies plotted across sequencing reads from concurrent 546-bp deletion and various insertion conditions, targeting episomally encoded eGFP. These are based on single-end sequencing, with three replicates per experiment, all sequenced on one run, overlaid. Note that only one of the two 3′-DNA-flaps is covered by the sequencing read in amplicons lacking the deletion (labeled as ‘wild-type’). Locations within read corresponding to insertions at deletion junction are highlighted between the nick-site (black dotted line) and end of insertion (red dotted line). Y-axis scaling is different for each plot. (7B) Same as (7A), but for experiments targeting a genomically integrated copy of eGFP. (7C) The percentage of reads containing the programmed deletion that also contain the programmed insertion. Similar to FIG. 2F, but for experiments targeting a genomically integrated copy of eGFP. Error bars represent standard deviation for at least three transfection replicates.

FIGS. 8A-8D. Quantifying deletion efficiency and error frequency on native HPRT1 gene. (8A, 8B) Insertion, deletion and substitution error frequencies plotted across sequencing reads from: (8A) 118-bp or 252-bp deletion on HPRT1 using the Cas9/paired-gRNA strategy and (B) 118-bp or 252-bp deletion on HPRT1 using the PRIME-Del strategy. Sequencing reads aligning to the ‘deletion’ reference for HPRT1 condition are based on paired-end sequencing, while all the other conditions are based on the single-end sequencing. Each experiment has three replicates sequenced on one run, overlaid. Note that only one of the two 3′-DNA-flaps is covered by the sequencing read in amplicons lacking the deletion (labeled as ‘wild-type’) and that y-axis scaling is different for each insertion, deletion and substitution plots. (8C, 8D) Droplet fluorescence level in Droplet digital PCR (ddPCR) assay for: (C) 118-bp deletion and (D) 252-bp deletion. Ratio of FAM-positive droplets (detecting precise-deletion; upper panels) to HEX-positive droplets (detecting genomic DNA concentration; bottom panels) was used for measuring deletion efficiencies with PRIME-Del (left three wells) and Cas9/paired-gRNA (middle three wells) methods. For each probe set, negative control (NTC) was performed to ensure specific signal from precise deletion. It is noted that the separation is less clear (with more substantial ‘raining’ patterns between negative and positive levels) in the FAM channel compared to HEX channel, possibly due to inefficient PCR amplification within the droplet. This phenomenon is more pronounced in Cas9/paired-gRNA samples, possibly due to annealing of FAM-probe to deletion junction with short (1 bp) mismatches as described previously (Watry et al. Rapid, precise quantification of large DNA excisions and inversions by ddPCR, Scientific Reports 2020).

FIGS. 9A-9H. Rare long insertions upon PRIME-Del editing of the HPRT1 exon 1. (9A) paired-end sequencing was performed of amplicons derived from the PRIME-Del-edited HPRT1 locus to bidirectionally cover the deletion junction and facilitate removal of PCR duplicates using 15-bp UMI sequences. This revealed recurrent long insertions that upon inspection appear to be chimeras of the two 3′ flap sequences, with overlap at their GC-rich ends (highlighted in purple). Shown here is a representative insertion from the 118-bp deletion condition. Sequence identifiers are indicted. (9B-9D) Histograms of insertion sequence lengths for HPRTI 118-bp deletion with Cas9/paired-gRNA (9B), HPRTI 118-bp deletion with PRIME-Del (9C), or eGEP 546-bp deletion with PRIME-Del (9D). Red vertical lines denote the mean insertion lengths. (9E) Same as (9A), but representative insertion from the 252-bp deletion condition, also a chimera of the two 3′ flap sequences, with overlap at their GC-rich ends. Sequence identifiers are indicted. (9F, 9G) Histogram of insertion sequence lengths for HPRTI 252-bp deletion with PRIME-Del (9F) or Cas9/paired-gRNA (9G). (914) Potential mechanism of long insertions with PRIME-Del. GC-rich ends of 3′-flaps of paired pegRNAs (GCCCT in case of 118-bp deletion and CGGC in case of 252-bp deletion) anneal to one another, or to another GC-rich stretch, resulting in insertion upon repair.

FIGS. 10A-10E. PRIME-Del efficiency and accuracy depends on homology arm lengths. (10A) Paired pegRNAs can be designed with different RT-template lengths, which effectively alters the homology arm lengths to guide the editing in PRIME-Del. (10B, 10C) Deletion efficiencies from using different homology arm lengths for (109) 118-bp and (10C) 252-bp deletions of HPRTI exonl, normalized to the standard designs (32-bps RT templates; used in FIGS. 3A-3G). (mean±SD over n=3 transfection replicates). Using a non-homologous RT template sequence from making 546-bp deletion on eGFP (used in FIGS. 1A-2F; denoted as 30/30 eGFP) does not result in deletion. (10D, 10E) Long-insertion frequency in PRIME-Del from using different homology arm lengths for (10D) 118-bp and (10E) 252-bp deletions of HPRTI exonl, normalized to the standard designs. (mean±SD over n=3 transfection replicates).

FIGS. 11A-11C. Pooled deletion using PRIME-Del. (11A) Cartoon representation of four deletions programmed within the HPRTI gene, pooled together for transfection. (11B) Deletion efficiencies and error frequencies for 3 overlapping-deletions (118, 252 and 469 bps) on HPRTI gene using PRIME-Del in HEK293T cells. Three transfection replicates are plotted separately. (11C) 1064-bp deletion efficiencies compared between single-deletion (left three wells) and pooled PRIME-Del (middle three wells). Estimated editing efficiencies for 1064-bp deletion in pooled PRIME-Del are 1.7%, 1.9% and 2.0% for three transfection replicates.

FIGS. 12A-12F. Extending the editing time window enhances prime editing and PRIME-Del efficiency. (12A) Schematic for stably expressing both Prime Editor-2 enzyme and pegRNAs via two-step genome integration. (12B, 12C) Editing efficiencies measured for the 118-bp and 252-bp deletions at genomic HPRT1 exon 1 using PRIME-Del (paired-pegRNA construct) or CTT-insertion using prime editing (single-pegRNA construct) in K562(PE2) cells (12B) or HEK293T(PE2) cells (12C), as a function of time after initial transduction of pegRNA(s). (mean±SD over n=3 transfection replicates) (12D) Editing efficiencies measured for the 118-bp and 252-bp deletions at genomic HPRT1 exon 1 using PRIME-Del (paired-pegRNA construct) or CTT-insertion using prime-editing (single-pegRNA construct), as a function of time after initial transduction of pegRNA(s). Plasmids bearing paired-pegRNAs and Prime Editor-2 enzyme were transfected 3 times (days 0, 9, 18; highlighted in yellow) into Prime Editor-2 enzyme-expressing HEK293T cells. (mean±SD over n=3 transfection replicates) (12E) Same as (12A), but first with integration of pegRNAs to PE2-expressing HEK293T via piggyBAC transposon system on Day 0 (highlighted in green), followed by two additional transfections of plasmid bearing Prime Editor-2 enzyme only on Day 9 and 18 (highlighted in yellow). (mean±SD over n=3 transfection replicates) (12F) Second replicate for experiment shown in (12C), where deletion efficiencies are measured for the 118-bp and 252-bp deletions at HPRT1 exon 1 using PRIME-Del as a function of time after initial transduction of pegRNA(s). (mean±SD over n=3 transfection replicates).

FIG. 13 schematically illustrates an embodiment of PRIME-Del configured to insert a sequence between the break sites after removal of the intervening sequence. The 3′ flaps have the sequence to be inserted, with each flap (A and a) having the sequence in reverse complementary format such that they anneal during the repair step, resulting in inserted dsDNA sequence after the repair step. The regions of correspondence are indicated with letter designations A/a.

FIG. 14 schematically illustrates an embodiment of PRIME-Del configured to circularize a fragment of dsDNA. The first target sequence (top strand) is disposed in a more 3′ location along the sense strand than the reverse complement sequence in the sense strand corresponding to the second target sequence of the antisense sense strand (bottom strand). In this embodiment, the first 3′ overhang flap (B) and the second 3′ overhang flap (a) point outwardly and away from each other. In this orientation, the repair results in excision of dsDNA fragment(s) on either side of the single-stranded breaks, preserving the portion of the dsDNA sequence disposed between the first single-stranded break of the sense strand and second single stranded break in the second strand. In this illustrated embodiment, each 3′ flap (B and a) contains sequence that is complementary to the preserved dsDNA region targeted by the other pegRNA, as in FIG. 1C, although additional insertion sequence can be included or substituted entirely, such as in FIGS. 2A and 13, respectively.

### DETAILED DESCRIPTION

Current methods to delete genomic sequences are based on CRISPR-Cas9 and pairs of single-guide RNAs (sgRNAs), but can be inefficient and imprecise, with errors including small indels as well as unintended large deletions and more complex rearrangements. This disclosure provides a prime editing-based method, called “PRIME-Del” that induces a deletion using a pair of prime editing sgRNAs (pegRNAs) that target opposite DNA strands. The pegRNAs program not only the sites that are nicked but also the outcome of the repair. As described in more detail below, PRIME-Del achieves markedly higher precision than CRISPR-Cas9 and sgRNA pairs in programming deletions up to 10 kb with 1-30% editing efficiency. PRIME-Del can also be used to couple genomic deletions with insertions, enabling deletions whose junctions do not fall at protospacer-adjacent motif (PAM) sites. Finally, extended expression of prime editing components can substantially enhance efficiency without compromising precision. PRIME-Del will be broadly useful for reliable, precise, and flexible programming of genomic deletions and insertions, for epitope tagging, and for programming genomic rearrangements.

In accordance with the foregoing, in one aspect the disclosure provides a method of editing a double stranded DNA (dsDNA) molecule. The target dsDNA can be characterized as having a sense strand and antisense strand, which have sequences that are typically reverse complements of each other. The opposing strands mutually hybridize via Watson-Crick base pairing, conferring stability of the dsDNA molecule in the canonical double helix configuration. Any dsDNA molecules can be targeted with the present methods. Exemplary dsDNA is genomic DNA from any cell, organism, or virus. In somebody embodiments, the dsDNA is genomic DNA from a human cell. The terms sense and antisense can be assigned arbitrarily to either strand and, unless indicated otherwise, are used simply to differentiate the opposing strands from each other.

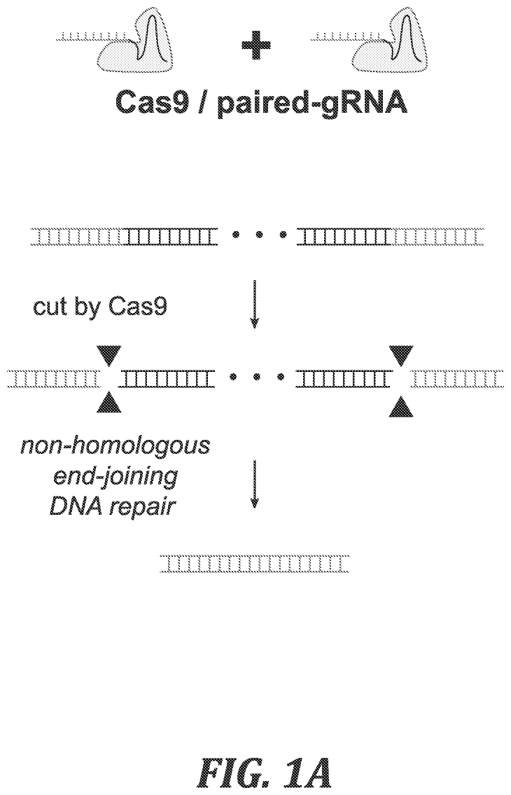

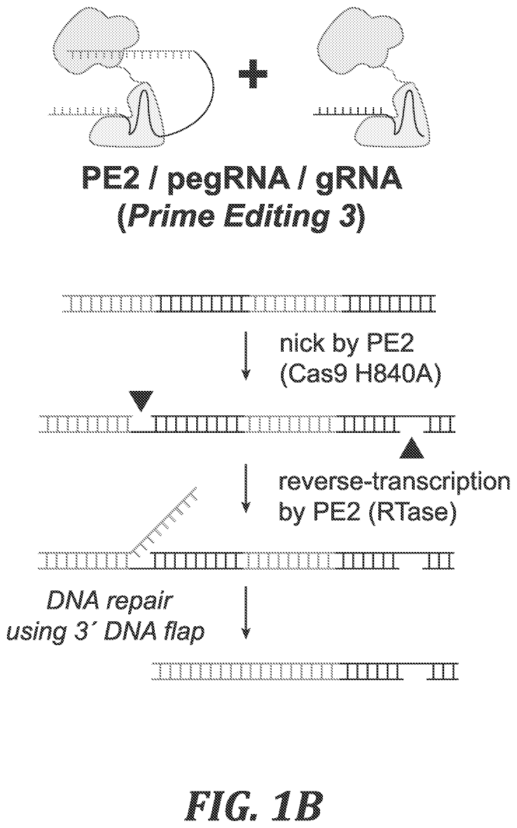

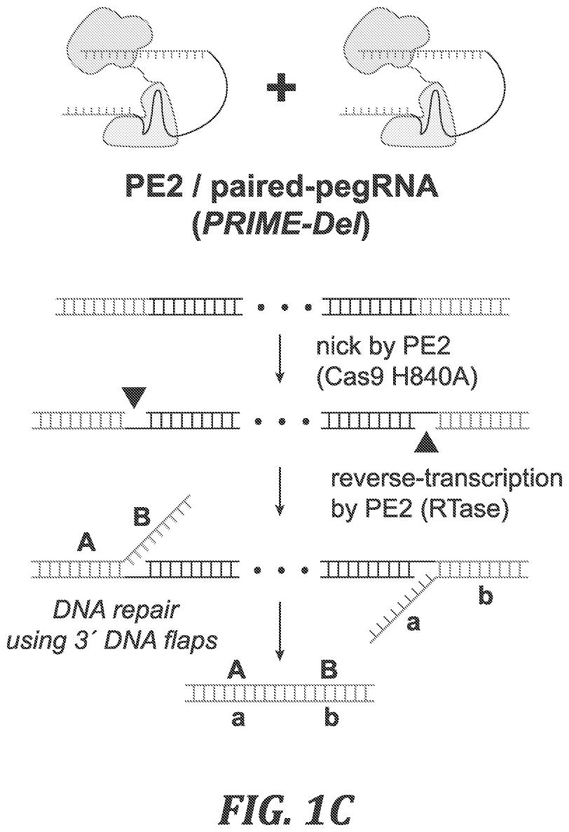

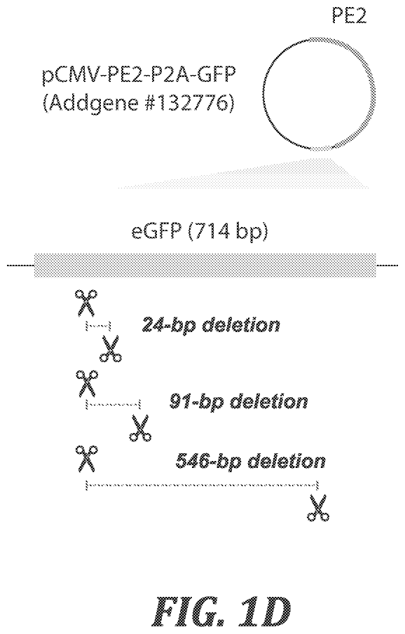

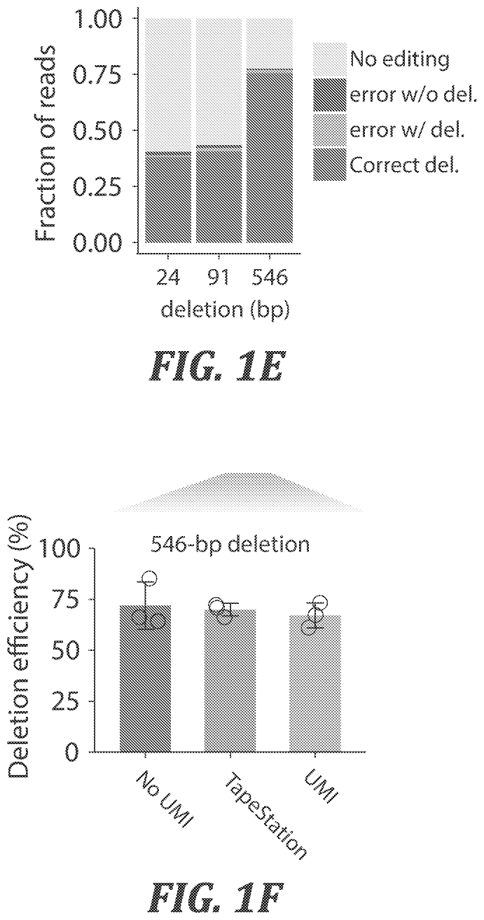

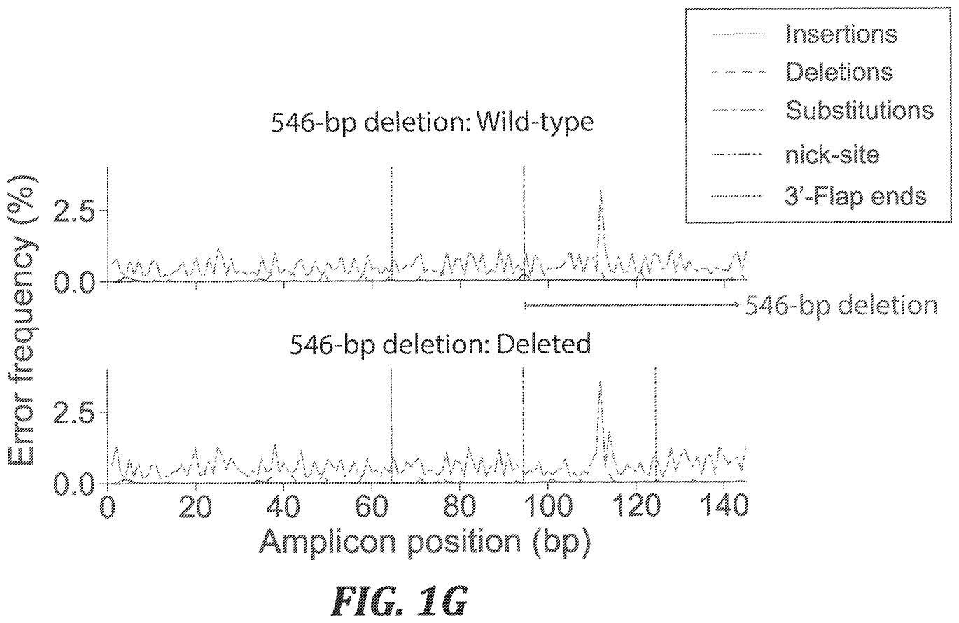

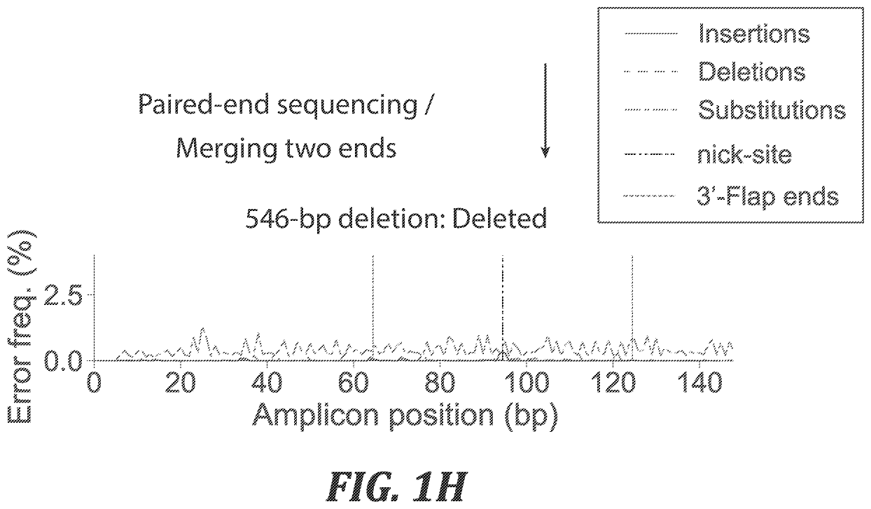

The method comprises contacting the dsDNA molecule with at least one pair of editing complexes. Each editing complex of the pair is based on prime editing constructs, previously disclosed by Anzalone et al. Search-and-replace genome editing without double-strand breaks or donor DNA. Nature 576, 149-157 (2019) and Lin, Q. et al. Prime genome editing in rice and wheat. Nat. Biotechnol. 38, 582-585 (2020), each of which is expressly incorporated herein by reference in its entirety. As explained in more detail below and illustrated in FIG. 1B, prime editing utilizes an editor enzyme with nickase capability fused to a reverse transcriptase. The prime editing construct further includes a 3′-extended sgRNA, also referred to as a prime-editing sgRNA or pegRNA). When coupled, the pegRNA confers binding specificity to a target sequence and the fusion editor nicks (i.e., causes a break in the phospho-diester linkage joining neighboring nucleotides in) one strand of the dsDNA molecule. A 3′ single stranded DNA flap is attached to the nicked site by reverse transcription of a portion of the pegRNA by the transcriptase domain of the fusion editor protein.

In the disclosed method, however, a pair of editing complexes are used, each of which are specifically targeted to portions of the dsDNA on opposing strands. An overview illustrating some embodiments of the approach is provided in FIG. 1C. In particular, the dsDNA is contacted with a first editing complex and a second editing complex. The first editing complex is specific for a first target sequence on the sense strand of the dsDNA molecule and a second editing complex specific for a second target sequence on the antisense strand of the dsDNA molecule. The term “specific for” means that the editing complex contains a structural element (e.g., RNA sequence) that can selectively bind (e.g., hybridize to) the target sequence under normal conditions. The first editing complex and the second editing complex each independently comprise a fusion editor protein and an extended guide RNA molecule associated therewith.

It is noted that for purposes of simplicity this description addresses the components of the editing complexes, their implementation, and their use in the general context of a single pair of editing complexes. However, this disclosure also encompasses embodiments comprising use of a plurality of editing complex pairs. For these embodiments, it will be understood that each pair of editing complexes can be distinct from other pairs of editing complexes, thus leading to different targeting and/or editing functionality. For example, the structure that confers specific targeting of the editing complexes (described below) can vary among the pairs of editing complexes. The result is implementation of multiple, distinct edits at multiple target locations in the same dsDNA molecule or in different dsDNA molecules in the same environment (e.g., in different chromosomes of the same cell). In view of the following description, it will become apparent how to implement multiplexed editing with multiple pairs of editing complexes. For example by pooling just distinct extended guide RNA molecules (or nucleic acid sequences encoding the extended guide RNA molecules) such that they can complex with the fusion editor proteins, where the fusion editor proteins can all be the same or different.

Generally described, fusion editor proteins each comprise a functional nickase domain and a functional reverse transcriptase domain, in any orientation with respect to each other so long as they retain their functional capacities (as described below). It will be understood that the respective functional nickase domains and a functional reverse transcriptase domains, with respect to the first and second editing complex, can be the same or different as long as they retain their functional capacities. The general organization of the respective extended guide RNA molecules includes a guide domain containing a sequence that hybridizes to a desired target sequence in the dsDNA and an extended domain at the 3′ end with a desired sequence to be incorporated into the edited DNA or otherwise to facilitate a desired mode of repair. In some embodiments, the first and/or second extended domain comprises two subdomains. The first subdomain comprises a primer-binding sequence (PBS), that hybridizes with the nicked strand. The first subdomain is at the 3′-end of the extended domain (and typically the entire extended guide RNA molecule as well). The second subdomain comprises a reverse-transcription template (RTT), which serves as the template for the 3′ overhang such that it is reverse-transcribed from RNA to DNA to add the 3′-overhang. The RTT is between the PBS and the guide domain. The RTT sequence is the reverse-complement of the 3′ overhang.

In many implementations, the respective extended guide RNA molecules of the first editing complex and the second editing complex contain different sequences depending on their respective target sequences or 3′ end sequences. With more particularity, the extended guide RNA molecule of the first editing complex comprises a first guide domain with a first sequence that hybridizes to the first target sequence and a first extended domain at the 3′ end. The extended guide RNA molecule of the second editing complex comprises a second guide domain with a second sequence that hybridizes to the second target sequence and a second extended domain at the 3′ end.

Upon specific binding of the first editing complex and second editing complex to their respective targets in the dsDNA molecule, the method comprises permitting the functional nickase domain of the first editing complex and the functional nickase domain of the second editing complex to create a first single-stranded break and second single stranded break (e.g., nick) in opposite strands of the dsDNA molecule at the first target sequence and second target sequence, respectively. In some embodiments, the functional nickase domain of the first editing complex nicks the sense strand within the first target sequence (e.g., within about 3 bases upstream of a protospacer adjacent motif (PAM) sequence). Similarly, in some embodiments, the functional nickase domain of the second editing complex nicks the anti-sense strand within the second target sequence (e.g., within about 3 bases upstream of a protospacer adjacent motif (PAM) sequence).

After the first and second single stranded breaks are induced by the first and second editing complexes (i.e., via the respective nickase domains) on the sense and anti-sense strands, respectively, the method comprises permitting the functional reverse transcriptase domain of the first editing complex to generate a first 3′ overhang from the first single stranded break using the first extended domain as template. Similarly, the method comprises permitting the functional reverse transcriptase domain of the second editing complex to generate a second 3′ overhang from the second single stranded break using the second extended domain as template.

After extension of the first and second 3′ overhangs at the first and second nicks, respectively, the dsDNA molecule is repaired. The result of the repair can depend on the relative position of the first and target sequences, and therefore the relative orientation first and second breaks and resulting positioning of the first and second 3′ overhangs. To addresses these configuration, the relative positions can be expressed in the context of the 5′ to 3′ axis of the sense strand. In one embodiment, the first target sequence is disposed in a more 5′ location along the sense strand than the reverse complement sequence in the sense strand corresponding to the second target sequence of the antisense sense strand. This embodiment is illustrated in FIG. 1C. In this embodiment, the first 3′ overhang and the second 3′ overhang point inwardly and towards each other. In this orientation the dsDNA repair results in excision of the portion of the dsDNA originally disposed between the first single-stranded break of the sense strand and second single stranded break in the second strand. The first 3′ overhang and the second 3′ overhang are integrated into the repaired dsDNA molecule. An embodiment of this repair scheme is illustrated in FIG. 1C. In some embodiments, both 3′ overhang can be further extended via innate cellular DNA damage repair capabilities during this process.

In an alternative embodiment, the first target sequence is disposed in a more 3′ location along the sense strand than the reverse complement sequence in the sense strand corresponding to the second target sequence of the antisense sense strand. In this embodiment, the first 3′ overhang and the second 3′ overhang point outwardly and away from each other. In this orientation, the repair results in excision of dsDNA fragment(s) on either side of the single-stranded breaks, preserving the portion of the dsDNA sequence disposed between the first single-stranded break of the sense strand and second single stranded break in the second strand. The first 3′ overhang and the second 3′ overhang can be integrated back into the repaired dsDNA molecule, thereby circularizing the portion of the dsDNA sequence disposed between the first single-stranded break of the sense strand and second single stranded break in the second strand. FIG. 14 is a schematic representing an embodiment of this circularization process using PRIME-del.

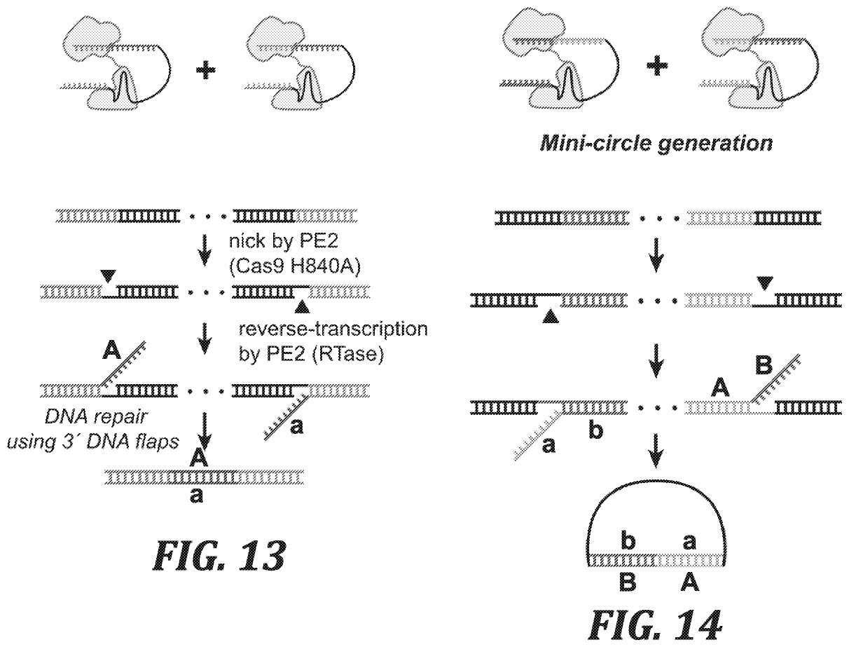

In some embodiments, the first 3′ overhang and the second 3′ overhang each comprise nucleic acid sequences that are reverse complements of each other and that hybridize in the repairing step. A representation of this embodiment is provided in FIG. 13. The portion of the dsDNA previously present between the two single stranded breakpoints is excised during the repair. The two overhangs with reverse complementary sequences hybridize and result in a double stranded molecule that is functionally inserted in the dsDNA in place of the excised portion. This results in an insertions sequence disposed between the original dsDNA molecule sequence “upstream” of the first single stranded break and the original dsDNA molecule sequence “downstream” (with respect to sense strand orientation) of the second single stranded break.

In other embodiments, the first 3′ overhang comprises a first repair domain with a sequence that corresponds to a sequence adjacent to and immediately 5′ to the second 3′ overhang in the antisense strand. Similarly, the second 3′ overhang comprises a second repair domain with a sequence that corresponds to a sequence adjacent to and immediately to the first 3′ overhang in the sense strand. In this embodiment, during the repair step the first 3′ overhang and the second 3′ overhang in the opposing strand reach past each other and hybridize to the remaining dsDNA portion adjacent to the opposing break points. A version of this embodiment is illustrated in FIG. 1C.

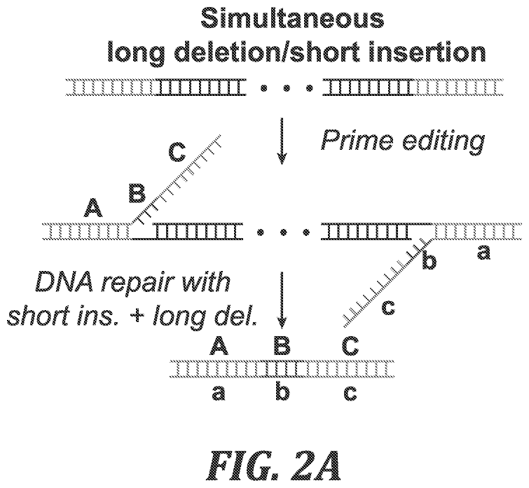

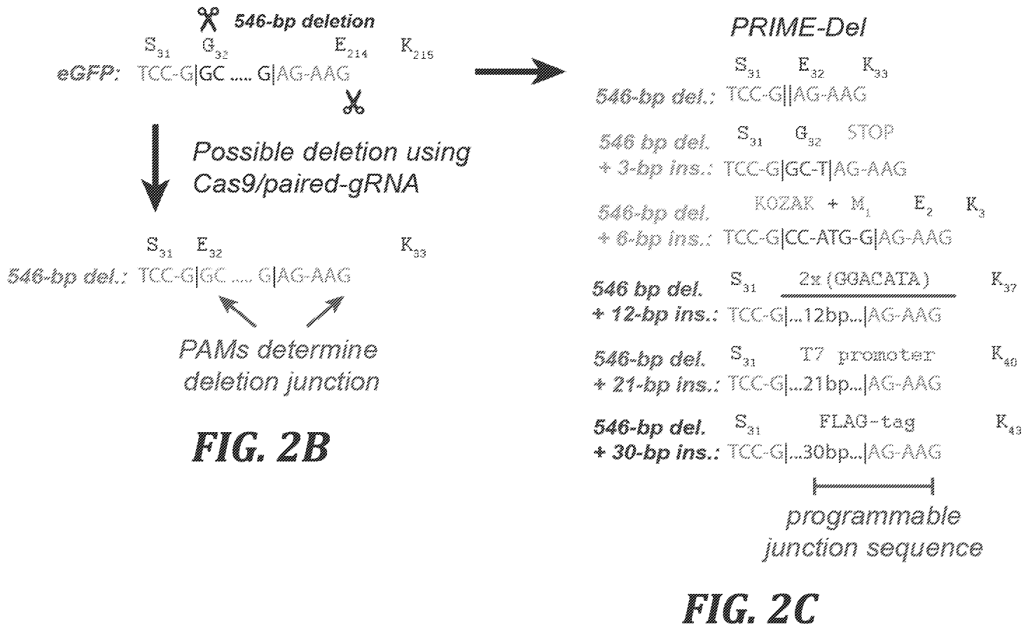

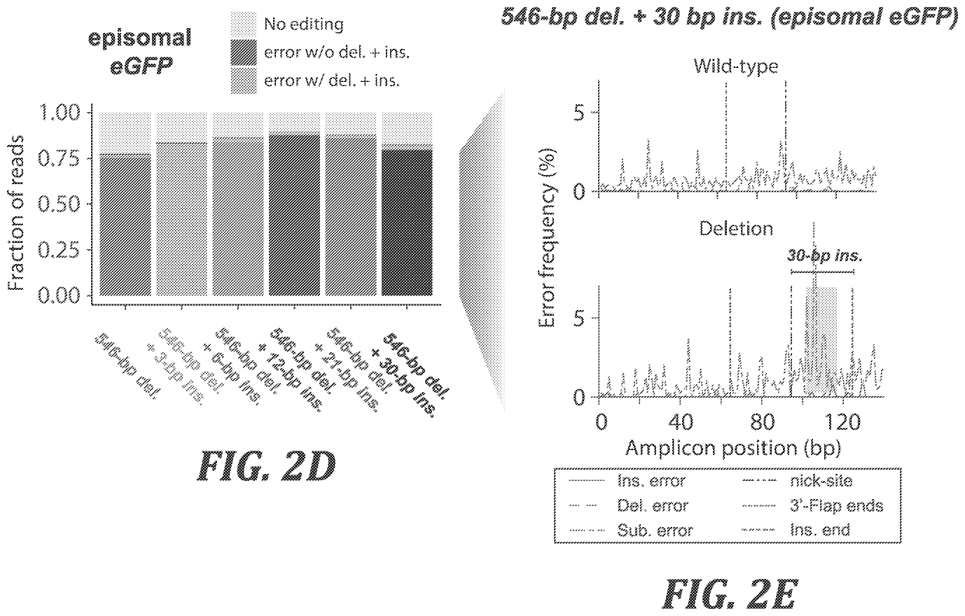

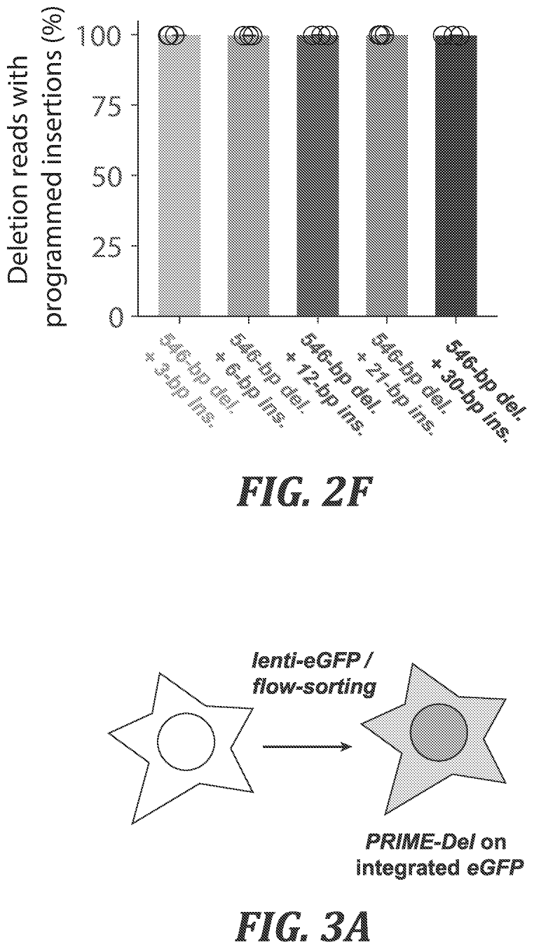

In a further embodiment, the overhang sequences can comprise multiple sequences, e.g., sequence that corresponds to a portion of the dsDNA that facilitates repair and sequence constituting a new sequence that will be incorporated as a new sequence. For example, the first 3′ overhang can further comprise an insertion sequence disposed 5′ to the first repair domain. Similarly, the second 3′ overhang comprises a corresponding insertion sequence, i.e., that is the reverse complement of the insertion sequence in the first 3′ overhang, and which is disposed 5′ to the second repair domain within the second 3′ overhang. During repair, the two insertion sequence domain hybridize. The first repair domain of the first 3′ overhang reaches past the second break point and hybridizes to the remaining dsDNA portion adjacent to the second breakpoint. Similarly, the second repair domain of the second 3′ overhang reaches past the first break point and hybridizes to the remaining dsDNA portion adjacent to the first breakpoint. An example of this embodiment is illustrated in FIG. 2A.

The method comprises other variations that can be implemented by design of the overhang sequences. For example, the method can be implemented in a manner that inverts the orientation sequence displeased between the first and second target domains. In one embodiment to implement such an inversion, the first 3′ overhang comprises a first repair domain with a sequence that corresponds to a sequence immediately 3′ to the second single stranded break (i.e., in the anti-sense strand). Similarly, the second 3′ overhang comprises a second repair domain with a sequence that corresponds to a sequence immediately 3′ to the first single stranded break (e.g., in the sense strand). Stated otherwise, the 3′ overhangs each contain a sequence that hybridizes to the opposing end of the intervening dsDNA fragment. As a result, the repairing step results in an inversion of the sequence corresponding to the portion of the dsDNA originally disposed between the first single-stranded break and second single stranded break. In some embodiments, the first repair domain has a sequence that is identical (or substantially identical) to a sequence immediately 3′ to the second single stranded break. Similarly, in some embodiments, the second repair domain has a sequence that is identical (or substantially identical) to a sequence immediately 3′ to the first single stranded break.

In some embodiments, the method can be used to insert a DNA fragment (“insertion DNA fragment”) from an exogenous source between the first and second target domains in the target dsDNA molecule. The insertion DNA fragment being inserted can be a linear DNA fragment or be derived from a circular DNA molecule. To facilitate the insertion, the first 3′ overhang comprises a first repair domain with a sequence corresponding to a first domain of the insertion DNA fragment. Similarly, the second 3′ overhang comprises a second repair domain with a sequence corresponding to a second end domain of the insertion DNA fragment. The first domain and second domain can be end domains at opposite ends of the insertion DNA fragment. Alternatively, one or both of the first domain and second domain are at distinct sites, e.g., internal sites, within a larger dsDNA molecule that ultimately contains the insertion DNA fragment. In this alternative embodiment, the first domain and second domain define the ends of the portion of insertion DNA fragment within the larger exogenous dsDNA source molecule.

As indicated below, the various embodiments of the method can be leveraged to delete a wide range of internal dsDNA fragments sizes from a target dsDNA molecule. The disclosed method can be used to delete intervening sequence of almost any length, for example from as shorts as about 5 or 10 nucleotides to a long as about 1 million nucleotides or more, although the reaction may exhibit some reduction in efficiency at the longer deletions. To illustrate, in some embodiments, the portion of the dsDNA originally disposed between the first single-stranded break and second single stranded break that is excised is from about 5 nucleotides to about 1 million nucleotides, from about 10 nucleotides to about 900,000 nucleotides, from about 10 nucleotides to about 800,000 nucleotides, from about 10 nucleotides to about 700,000 nucleotides, from about 10 nucleotides to about 700,000 nucleotides, from about 10 nucleotides to about 600,000 nucleotides, from about 10 nucleotides to about 500,000 nucleotides, from about 10 nucleotides to about 400,000 nucleotides, from about 10 nucleotides to about 300,000 nucleotides, from about 10 nucleotides to about 200,000 nucleotides, from about 10 nucleotides to about 100,000 nucleotides, from about 10 nucleotides to about 90,000 nucleotides, from about 10 nucleotides to about 80,000 nucleotides, from about 10 nucleotides to about 70,000 nucleotides, from about 10 nucleotides to about 60,000 nucleotides, from about 10 nucleotides to about 50,000 nucleotides, from about 10 nucleotides to about 40,000 nucleotides, from about 10 nucleotides to about 30,000 nucleotides, from about 10 nucleotides to about 20,000 nucleotides, from about 10 nucleotides to about 10,000 nucleotides, from about 10 nucleotides to about 9,000 nucleotides, from about 10 nucleotides to about 8,000 nucleotides, from about 10 nucleotides to about 7,000 nucleotides, from about 10 nucleotides to about 6,000 nucleotides, from about 10 nucleotides to about 5,000 nucleotides, from about 10 nucleotides to about 4,000 nucleotides, from about 10 nucleotides to about 3,000 nucleotides, from about 10 nucleotides to about 2,000 nucleotides, from about 10 nucleotides to about 1,000 nucleotides, or any subrange therein. For example, the portion of the dsDNA originally disposed between the first single-stranded break and second single stranded break that is excised is at least 5 nucleotides in length, such as about 5, 6, 7, 8, 9, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, 550, 600, 650, 700, 750, 800, 850, 900, 950, 1000, 1500, 2000, 2500, 3000, 3500, 4000, 4500, 5000, 5500, 6000, 6500, 7000, 7500, 8000, 8500, 9000, 9500, 10,000, 20,000, 30,000, 40,000, 50,000, 60,000, 80,000, 90,000, 100,000, 200,000, 300,000, 400,000, 500,000, 600,000, 700,000, 800,000, 900,000, 1,000,000 or more nucleotides, or any number or range therein, in length.

In some embodiments, the first guide domain and second guide domain are independently between about 15 and about 200 nucleotides long. In exemplary, non-limiting examples, the first guide domain and second guide domain are independently between about nucleotides long, between about 15 and 150 nucleotides long, between about and 125 nucleotides long, between about 15 and 75 nucleotides long, between about 15 and 50 nucleotides long, between about 15 and 40 nucleotides long, between about 15 and nucleotides long, between about 15 and 25 nucleotides long, between about 15 and 20 nucleotides long, between about 20 and 200 nucleotides long, between about 20 and 175 nucleotides long, between about 20 and 150 nucleotides long, between about 20 and 125 nucleotides long, between about 20 and 100 nucleotides long, between about 20 and 75 nucleotides long, between about 20 and 50 nucleotides long, between about 20 and 40 nucleotides long, between about 20 and 30 nucleotides long, between about 20 and 25 nucleotides long, between about 25 and 50 nucleotides long, between about 25 and 40 nucleotides long, between about 25 and 30, nucleotides long, and any number or subrange therein. Illustrative lengths include about 18, 19, 20, 21, 22, 23, 24, 25, 26, 27, 28, 29, 30, 40, 45, 50, 55, 60, 65, 70, 75, 80, 85, 90, 95, 100, 125, 150, 175, 200 nucleotides long.

In some embodiments, one or both of the first and second guide domains is/are configured to be compatible with the first and second editing complex, respectively. In this context, “compatible” refers to the ability of the guide domain to be recognized by the fusion editor protein to form the editing complex. For example, in some embodiments the guide domain(s) can comprise one or more nucleotide residues that are modified with 2′-locked nucleic acids, peptide nucleic acids, or a similar functionally modified nucleic acid moiety. These illustrative modification and others are known to facilitate recognition and association with the fusion editor proteins in prime editing and are encompassed by the present disclosure.

The first extended domain and second extended domain can independently at least about 10 nucleotides long. Any practical upper limit to the length of either extended domain is likely to be imposed by the capacity of the functional reverse transcription domain in the prime-editing-based approach to create a 3′ overhang from the extended domain template. Such functional reverse transcription domains can readily reverse transcribe 1000-2000 nucleotide lengths. Thus, the extended domains can independently be between about 10 to about 2000 nucleotides in length. It may be more typical for the extended domains to be on the shorter end of the range for certain applications. Illustrative, nonlimiting ranges include between about 10 and 500 nucleotides long, between about 10 and 400 nucleotides long, between about 10 and 300 nucleotides long, between about 10 and 200 nucleotides long, between about 10 and 100 nucleotides long, between about 10 and 75 nucleotides long, between about 10 and 50 nucleotides long, between about 10 and 40 nucleotides long, between about 10 and 30 nucleotides long, and between about 10 and 20 nucleotides long, or any length or subrange therein. Illustrative lengths include about 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 85, 90, 95, 100, 125, 150, 175, 200 nucleotides long.

It will be appreciated that, in some embodiments, the first extended guide RNA molecule can and/or the second extended guide RNA molecule can be engineered to include additional functional domains. For example, the (first and/or second) extended guide RNA molecule can further comprise a domain that aids in the efficiency of 3′-overhang generation. In one embodiment, the extended guide RNA has incorporated structured RNA motifs at the 3′ terminus (i.e., in the extended domain, described herein) that enhance their stability and prevent degradation of the 3′ extension. Such “anti-degradation” structure motifs are described, for example, in Nelson, J. W., et al. Engineered pegRNAs improve prime editing efficiency. Nat Biotechnol pp. 1-9 (2021), incorporated herein by reference in its entirety, and include modified prequeosinel-1 riboswitch aptamer (evopreQ 1; Roth, A. et al. A riboswitch selective for the queuosine precursor preQ1 contains an unusually small aptamer domain. Nat. Struct. Mol. Biol. 14, 308-317 (2007); and Anzalone, A. V., et al. Reprogramming eukaryotic translation with ligand-responsive synthetic RNA switches. Nat. Methods 13, 453-458 (2016), each of which is incorporated herein by reference in its entirety) and pseudoknots (e.g., from Moloney murine leukemia virus).

The functional nickase domain can be any functional domain that catalyzes a single stranded break in a target dsDNA sequence. To illustrate, examples of the functional nickase domain encompassed by the disclosure include CRISPR-associated (Cas) enzyme, Pyrococcus furiosus Argonaute, and the like, or a functional nickase domain derived therefrom. In some embodiments, the nickase domain is derived from an enzyme that has been modified, such as to ablate double stranded nuclease functionality. Non-limiting examples of Cas enzymes useful in this aspect include Cas9 (dCas9 or nCas9), Cas12, Cas13, Cas3, CasED, and the like. See, e.g., Pauch, P, et al., CRISPR-Cas0 from huge phages is a hypercompact genome editor, Science, 369(6501):333-337 (2020), and WO 2020/191242, each of which is incorporated herein by reference in its entirety. A plasmid sequence encoding a useful Cas9 (with H804A modification for nickase capability) and M-MLV-rt with 5 point mutations is available at Addgene depository, catalogue No. 132775. Other useful Cas9 sequences, structures, and optimizations useful for this disclosure are known in the art Cas9 nuclease sequences and structures are well known to those of skill in the art (see, e.g., Ferretti el al. Complete genome sequence of an Ml strain of Streptococcus pyogenes, Proc. Natl. Acad. Sci. U.S.A. 98:4658-4663(2001); Deltcheva E., et al. CRISPR RNA maturation by trans-encoded small RNA and host factor RNase III. Nature 471:602-607(2011); and Jinek M., et al. A programmable dual-RNA-guided DNA endonuclease in adaptive bacterial immunity. Science 337:816-821(2012), each of which is incorporated herein by reference in its entirety.) Additionally, Cas (e.g., Cas9) orthologs have been described in various species, including, but not limited to, S. pyogenes and S. thermophilus. As indicated, the nickase domain can comprise a modification to ensure that the domain does not impose double stranded breaks but rather single stranded breaks.

Exemplary modifications include having one (of multiple) nuclease domains in the enzyme domain (e.g., Cas9 nuclease) being inactivated, leaving only the ability to impose single stranded breaks.

The fusion editor domain also comprises a functional reverse transcriptase (RT) domain. The functional RT domain can be any functional domain that catalyzes reverse transcription reactions. “Reverse transcriptase” generally refers to a class of polymerases characterized as RNA-dependent DNA polymerases. Historically, reverse transcriptase has been used primarily to transcribe mRNA into cDNA which can then be cloned into a vector for further manipulation and many such enzyme (and functional domains thereof) are known and encompassed by this disclosure. For example, avian myeloblastosis virus (AMV) reverse transcriptase was the first widely used RNA-dependent DNA polymerase (Verma, Biochem. Biophys. Acta 473:1 (1977)). RNase H is a processive 5′ and 3′ ribonuclease specific for the RNA strand for RNA-DNA hybrids (Perbal, A Practical Guide to Molecular Cloning, New York: Wiley & Sons (1984)). Another reverse transcriptase which is used extensively in molecular biology is reverse transcriptase originating from Moloney murine leukemia virus (M-MLV). See, e.g., Gerard, G. R., DNA 5:271-279 (1986) and Kotewicz, M. L., et ah, Gene 35:249-258 (1985). M-MLV reverse transcriptase substantially lacking in RNase H activity has also been described. See, e.g., U.S. Pat. No.

Other exemplary, non-limiting embodiments the functional reverse transcriptase domain include, HIV RT, group II intron RT (TGIRT) (see, e.g., InGex, St. Louis, MO), superscript IV (e.g., from ThermoFisher Scientific, Waltham, MA) and the like, or a functional domains thereof. Anzalone, A. V. et al. Search-and-replace genome editing without double-strand breaks or donor DNA. Nature 576, 149-157 (2019), incorporated herein by reference in its entirety, describes a fusion protein that has functional nickase and RT domains that are encompassed by the present disclosure. For example, wild-type M-MLV RT and engineered M-MLV RT domains can be useful embodiments. Furthermore, engineered RT domains can improve the prime-editing and prime-deletion disclosed herein. WO 2020/191242, incorporated herein in its entirety, describes additional examples of useful RT domain. This disclosure contemplates the use of any such reverse transcriptases, variants, mutants, or fragments thereof.

In some embodiments, the fusion editor protein can comprise additional functional domains. For example, the additional functional domain can be a functional enzymatic domain, such as a DNA repair protein domain. Inclusion of a DNA repair domain in the fusion editor protein can enhance the efficiency of DNA repair after generation of the 3′ overhang. An illustrative, nonlimiting example of such a domain is the functional DNA-binding domain from Rad15, or homologs thereof. See, e.g., Song, M., et al. Generation of a more efficient prime editor 2 by addition of the Rad51 DNA-binding domain. Nat Commun 12, 5617 (2021), incorporated herein by reference in its entirety.

The disclosed method can be used to accomplish many modifications to a specifically targeted dsDNA molecule, such as to accomplish a deletion, deletion combined with an insertion, an inversion of intervening sequence, a translocation of sequence (e.g., interchromosomal rearrangements), programming frame retention into the sequence, accessing a deletion boundary that cannot be accessed with conventional CRISPR-based approaches because there is no appropriate PAM sequence. The disclosed method can be performed in a cell, for example in a cell maintained in culture. Alternatively, the aforementioned methods can be performed in vivo. For example, the method can be a therapeutic method comprising deletion of a genomic sequence, inverting a genomic sequence, interchromosomal rearrangement, and/or inserting a new sequence into a target region or site of the genome. In therapeutic embodiments, the compositions are formulated for appropriate administration (e.g., systemic) according to standard and known practices in the art.

The editing complexes can be delivered to the cells directly, or can be delivered/administered in the form of encoding nucleic acids incorporated into suitable vectors for cell delivery and expression. Thus, in some embodiments, the method comprises delivering one or more fusion editor protein-encoding and extended guide RNA molecule-encoding polynucleotides, such incorporated into one or more vectors, one or more transcripts thereof, and/or one or proteins transcribed therefrom, to a target cell. Appropriate viral and nonviral vector systems are known and can be implemented by persons of ordinary skill in the art. For example, exemplary non-viral vector delivery systems include DNA plasmids, RNA (e.g. a transcript of a vector described herein), naked nucleic acid, and nucleic acid complexed with a delivery vehicle, such as a liposome. Non-viral delivery of nucleic acids includes lipofection, nucleofection, microinjection, biolistics, virosomes, liposomes, immunoliposomes, polycation or lipidnucleic acid conjugates, naked DNA, artificial virions, and agent-enhanced uptake of DNA.

Viral vector delivery systems include DNA and RNA viruses, which have either episomal or integrated genomes after delivery to the cell. The use of RNA or DNA viral based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro, and the modified cells can optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. A variety of delivery and formulation strategies appropriate for implementation in the present methods with respect to the described editing complexes, or fusion editor and extended guide RNA components (or encoding nucleic acids) are described in WO 2020/191242, the entire contents of which are incorporated herein by reference.

In another aspect, the disclosure provides a kit. The kit comprises any combination of the compositions described herein. In some embodiments, the kit comprises a pair of distinct editing complexes (i.e., first and second editing complexes) as described herein, one or more nucleic acids encoding the first and second fusion editor proteins and/or the first and second extended guide RNA molecules, or one or more vectors comprising the nucleic acids. As described above, the first and second editing complexes are specific for a first and second target sequence on a target dsDNA molecule, by virtue of the first and second guide domains of the first and second extended guide RNA molecules, respectively. The first target sequence is on the sense strand of the target dsDNA and second target sequence is on the antisense strand of the dsDNA. The two target sequences are separated by an intervening sequence. The first editing complex and the second editing complex are configured to delete intervening sequence, to invert the intervening sequence, and/or inserting one or more new sequences at the first and/or second single stranded breaks induced by the first editing complex and the second editing complex in the target dsDNA molecule, as described above in more detail. The kit can also optionally comprise various buffers and reagents to facilitate the reactions described herein. For example, the kit can comprise dNTPs, RNase inhibitors, cofactors (e.g., MgCl₂), and the like.

In some embodiments the kit can include one or more containers containing the various components for performing the basic methods described herein. Each of the components of the kits, where applicable, can be provided in liquid form (e.g., a solution) or solid form (e.g., powdered or lyophilized). In some embodiments some of the components may be reconstitute able or processable, for example by the addition of a suitable solvent.

In some embodiment, the kit further comprises written indicia addressing how to perform the methods described herein.

Additional Definitions

Unless specifically defined herein, all terms used herein have the same meaning as they would to one skilled in the art of the present disclosure. Practitioners are particularly directed to Sambrook J., et al. (eds.), Molecular Cloning: A Laboratory Manual, 3rd ed., Cold Spring Harbor Press, Plainsview, New York (2001); Ausubel, F. M., et al. (eds.), Current Protocols in Molecular Biology, John Wiley & Sons, New York (2010); Ran, F. A., et al., Genome engineering using the CRISPR-Cas9 system, Nature Protocols, 8:2281-2308 (2013), and Jiang, F. and Doudna, J. A., CRISPR—Cas9 Structures and Mechanisms, Annual Review of Biophysics, 46:505-529 (2017) for definitions and terms of art.

The use of the term “or” in the claims is used to mean “and/or” unless explicitly indicated to refer to alternatives only or the alternatives are mutually exclusive, although the disclosure supports a definition that refers to only alternatives and “and/or.”

Following long-standing patent law, the words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like, are to be construed in an inclusive sense as opposed to an exclusive or exhaustive sense; that is to indicate, in the sense of “including, but not limited to.” Words using the singular or plural number also include the plural and singular number, respectively. Additionally, the words “herein,” “above,” and “below,” and words of similar import, when used in this application, shall refer to this application as a whole and not to any particular portions of the application. The word “about” indicates a number within range of minor variation above or below the stated reference number. For example, “about” can refer to a number within a range of 10%, 9%, 8%, 7%, 6%, 5%, 4%, 3%, 2%, or 1% above or below the indicated reference number.

The terms “subject,” “individual,” and “patient” are used interchangeably herein to refer to a mammal being assessed for treatment and/or being treated. In certain embodiments, the mammal is a human. The terms “subject,” “individual,” and “patient” encompass, without limitation, individuals having cancer or disease comprising a genetic aberration. While subjects may be human, the term also encompasses other mammals, particularly those mammals useful as laboratory models for human disease, e.g., mouse, rat, dog, non-human primate, and the like.

The term “treating” and grammatical variants thereof may refer to any indicia of success in the treatment or amelioration or prevention of a disease or condition (e.g., a cancer, infectious disease, or autoimmune disease), including any objective or subjective parameter such as abatement; remission; diminishing of symptoms or making the disease condition more tolerable to the patient; slowing in the rate of degeneration or decline; or making the final point of degeneration less debilitating.

The treatment or amelioration of symptoms can be based on objective or subjective parameters; including the results of an examination by a physician. Accordingly, the term “treating” includes the administration of the compounds or agents of the present disclosure to prevent or delay, to alleviate, to improve clinical outcomes, to decrease occurrence of symptoms, to improve quality of life, to lengthen disease-free status, to stabilize, to prolong survival, to arrest or inhibit development of the symptoms or conditions associated with a disease or condition (e.g., a cancer or genetic disease), or any combination thereof. The term “therapeutic effect” refers to the reduction, elimination, or prevention of the disease or condition, symptoms of the disease or condition, or side effects of the disease or condition in the subject.

As used herein, the term “nucleic acid” refers to a polymer of nucleotide monomer units or “residues”. The nucleotide monomer subunits, or residues, of the nucleic acids each contain a nitrogenous base (i.e., nucleobase) a five-carbon sugar, and a phosphate group. The identity of each residue is typically indicated herein with reference to the identity of the nucleobase (or nitrogenous base) structure of each residue. Canonical nucleobases include adenine (A), guanine (G), thymine (T), uracil (U) (in RNA instead of thymine (T) residues) and cytosine (C). However, the nucleic acids of the present disclosure can include any modified nucleobase, nucleobase analogs, and/or non-canonical nucleobase, as are well-known in the art. Modifications to the nucleic acid monomers, or residues, encompass any chemical change in the structure of the nucleic acid monomer, or residue, that results in a noncanonical subunit structure. Such chemical changes can result from, for example, epigenetic modifications (such as to genomic DNA or RNA), or damage resulting from radiation, chemical, or other means. Illustrative and nonlimiting examples of noncanonical subunits, which can result from a modification, include uracil (for DNA), 5-methylcytosine, 5-hydroxymethylcytosine, 5-formethylcytosine, 5-carboxycytosine b-glucosyl-5-hydroxy-methylcytosine, 8-oxoguanine, 2-amino-adenosine, 2-amino-deoxyadenosine, 2-thiothymidine, pyrrolo-pyrimidine, 2-thiocytidine, or an abasic lesion.

An abasic lesion is a location along the deoxyribose backbone but lacking a base. Known analogs of natural nucleotides hybridize to nucleic acids in a manner similar to naturally occurring nucleotides, such as peptide nucleic acids (PNAs) and phosphorothioate DNA.

Reference to sequence identity addresses the degree of similarity of two polymeric sequences, such as nucleic acid or protein sequences. Determination of sequence identity can be readily accomplished by persons of ordinary skill in the art using accepted algorithms and/or techniques. Sequence identity is typically determined by comparing two optimally aligned sequences over a comparison window, where the portion of the peptide or polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity. Various software driven algorithms are readily available, such as BLAST N or BLAST P to perform such comparisons.

Disclosed are materials, compositions, and components that can be used for, can be used in conjunction with, can be used in preparation for, or are products of the disclosed methods and compositions. It is understood that, when combinations, subsets, interactions, groups, etc., of these materials are disclosed, each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

Publications cited herein and the subject matter for which they are cited are hereby specifically incorporated by reference in their entireties.

### EXAMPLES

The following examples are set forth so as to provide those of ordinary skill in the art with a complete disclosure and description of how to make and use the various aspects and embodiments of the disclosure, and are not intended to limit the scope of what the inventors regard as their innovation nor are they intended to represent that the experiments below are all or the only experiments performed.

### Example 1

This Example describes the development of a prime editing-based method, referred to as PRIME-Del, which induces a precise deletion using a paired prime-editing gRNA (pegRNA) that targets the two opposite DNA strands.

### INTRODUCTION

Investigations were conducted to determine whether a pair of pegRNAs could be used to specify not only the sites that are nicked but also the outcome of the repair. It was demonstrated that, as a result of the novel approach, deletions longer than 100 bp can be programmed (FIG. 1C). This strategy, referred to as PRIME-Del, is demonstrated to induce the efficient deletion of sequences up to 10 kb in length with much higher precision than observed or expected with either the Cas9/paired-sgRNA or extant PE3-strategies. It is further shown that PRIME-Del can concurrently program short insertions at the deletion site. Concurrent deletion/insertion can be used to introduce in-frame deletions, to introduce epitope tags concurrently with deletions, and, more generally, to facilitate the programming of deletions unrestricted by the endogenous distribution of PAM sites. By filling these gaps, PRIME-Del expands toolkits to investigate the biological function of genomic sequences at single nucleotide resolution.

### Results & Discussion

PRIME-Del Induces Precise Deletions in Episomal DNA

The feasibility of the PRIME-Del strategy was tested by programming deletions to an episomally encoded eGFP gene. Pairs of pegRNAs were designed specifying 24-, 91-and 546-bp deletions within the eGFP coding region of the pCMV-PE2-P2A-GFP plasmid (Addgene #132776) (FIG. 1D). Each pair of pegRNAs was cloned into a single plasmid with separate promoters, the human U6 and H1 sequences (Gasperini, M. et al. CRISPR/Cas9-Mediated Scanning for Regulatory Elements Required for HPRT1 Expression via Thousands of Large, Programmed Genomic Deletions. Am. J. Hum. Genet. 101, 192-205 (2017)). HEK293T cells were transfected with eGFP-targeting paired-pegRNA and pCMV-PE2-P2A-GFP plasmids. DNA (including both genomic DNA and residual plasmid) was harvested from cells 4-5 days after transfection and PCR amplified the eGFP region. PCR amplicons were then sequenced to quantify the efficiency of the programmed deletion as well as to detect unintended edits to the targeted sequence.

Deletion efficiency was calculated as the number of reads aligning to a reference sequence of the intended deletion, out of the total number of reads aligning to reference sequences either with or without the deletion. Estimated deletion efficiencies ranged from 38% (24-bp deletion) to 77% (546-bp deletion), and were consistent across replicates (note: throughout this Example, the term ‘replicate’ is used to refer to independent transfections) (FIG. 1E). This result clearly indicates that the PRIME-Del strategy outlined in FIG. 1C can work. It is was possible that these were overestimates of efficiency due to the shorter, edited templates being favored by both PCR and Illumina-based sequencing, particularly for the 546-bp deletion, because it has the largest difference between amplicon sizes (766-bp vs. 220-bp for wild-type and deletion amplicons, respectively). To address this, the amplification was repeated on DNA from the 546-bp deletion experiment with a two-step PCR, first adding 15 bp unique molecular identifiers (UMIs) via linear amplification before a second, exponential phase. The addition of UMIs via linear PCR was intended to minimize PCR and sequencing biases in the estimates of deletion efficiencies (Kivioja, T. et al. Counting absolute numbers of molecules using unique molecular identifiers. Nat. Methods 9, 72-74 (2011)). PRIME-Del efficiency was assessed based on the sequencing data after collapsing of reads with identical UMIs, as well as on the product size distribution (Agilent TapeStation). A slight decrease in deletion efficiency was observed after duplicate removal, from 73% to 66%, comparable to the 70% efficiency measured on the TapeStation (FIG. 1F). These results suggest that the initial estimates of efficiency are only modestly impacted by size-dependent biases.

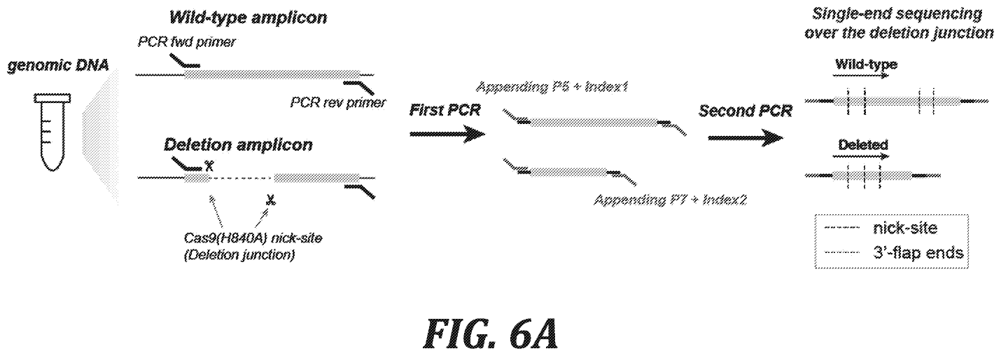

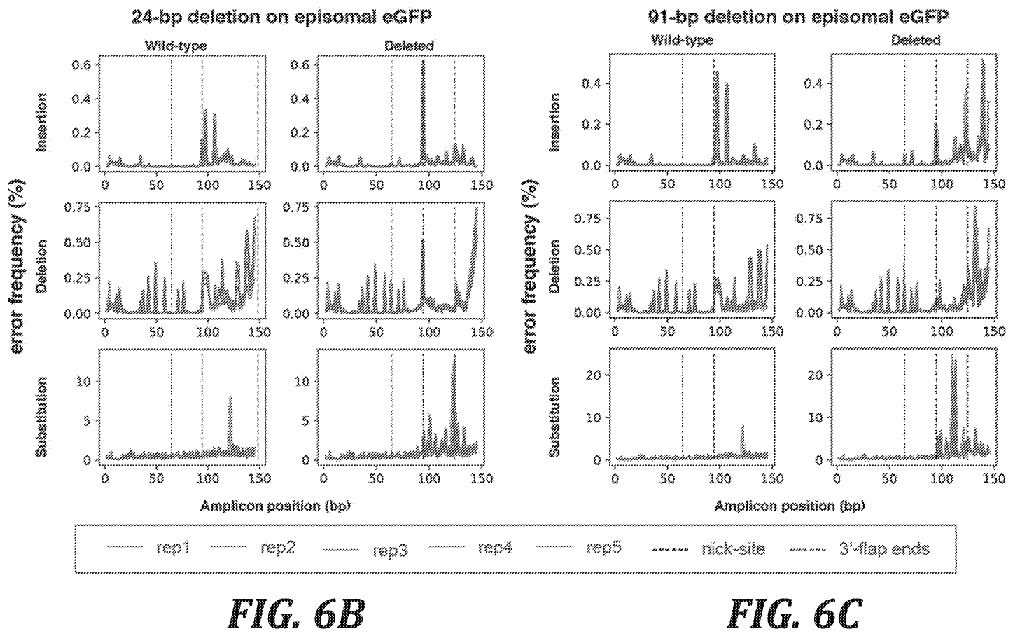

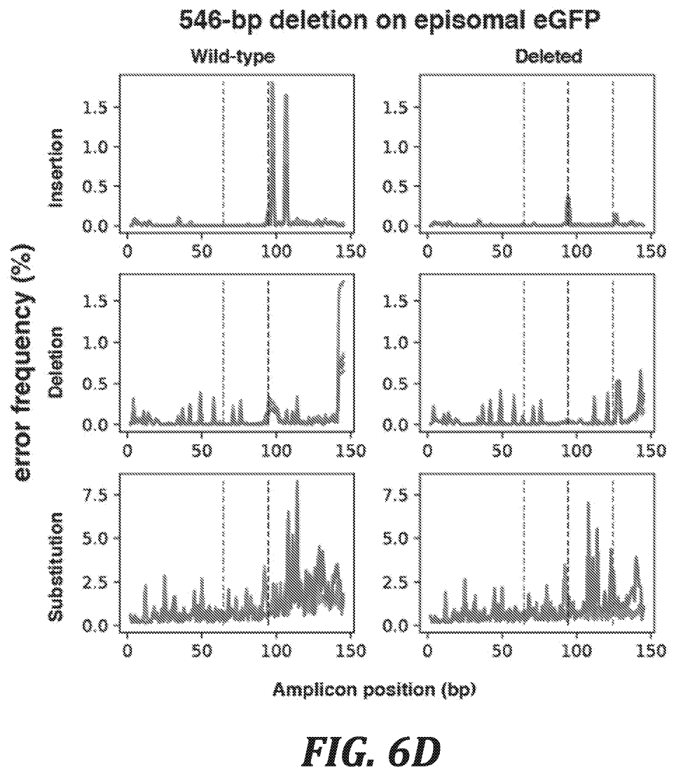

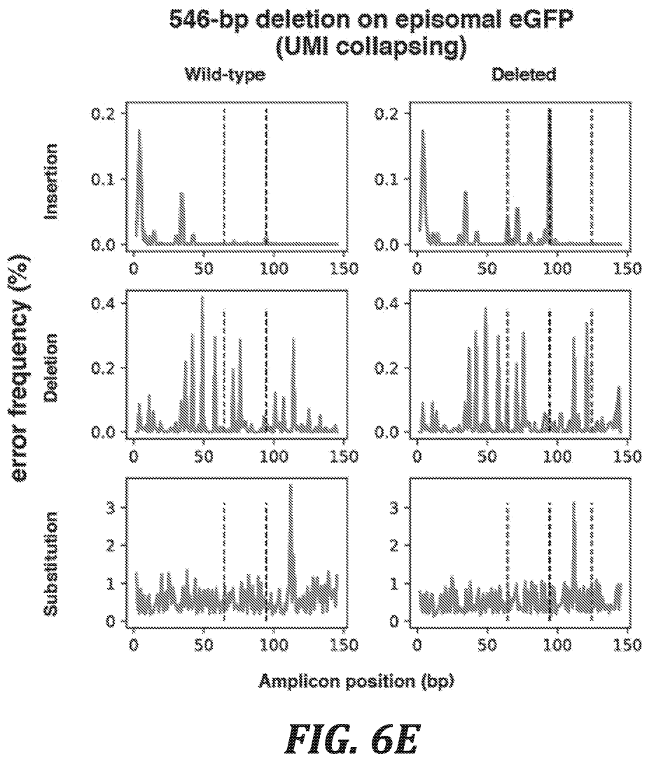

For most of these sequencing data, only a single read extended over the intended deletion site. As such, it was difficult to distinguish unintended editing outcomes (e.g. indels at the nick sites) from PCR or sequencing errors. To address this in part, frequencies of different classes of errors (substitutions, insertions, deletions) were plotted for sequences aligning either to the unedited sequence (FIG. 1G, top) or the intended deletion (FIG. 1G, bottom), along the length of the sequencing read. For all replicates of the three deletion experiments (FIGS. 6A-6E), these profiles showed low rates of substitutions and indels, with nearly identical profiles and no consistent increase in the rate of any class of error at either the positions of the Prime Editor-2 enzyme nick sites or 3° flap ends above 1%, particularly after collapsing by UMI (FIGS. 1G and 6E) or repeating sequencing with longer, paired-end sequencing reads (FIG. 1H).

Simultaneous Deletion and Short Insertion Using PRIME-Del

It was reasoned that because the homology sequences in the 3′-flaps program the deletion, PRIME-Del could potentially be used to concurrently introduce a short insertion at the deletion junction (FIG. 2A). The desired insertion would be encoded into the pair of pegRNAs in a reverse complementary manner, just 5′ to the deletion-specifying homology sequences. With the conventional strategy for programming deletions, i.e. with Cas9 and paired sgRNAs, the deletion junctions are determined by the sgRNA targets, the selection of which is limited by the natural distribution of PAM sites (FIG. 2B). Simultaneous deletion and short (less than 100 bps) insertion with PRIME-Del would offer at least three advantages over this conventional strategy. First, an arbitrary insertion of 1-3 bases could enable a reading frame to be maintained after editing, e.g. for deletions intended to remove a protein domain. Second, an arbitrary insertion could be used to effectively move one or both deletion junctions away from the cut-sites determined by the PAM, increasing flexibility to program deletions with base-pair precision. Third, insertion of functional sequences at the deletion junction could allow genome editing with PRIME-Del to be coupled to other experimental goals (e.g. protein tagging or insertion of a transcriptional start site).

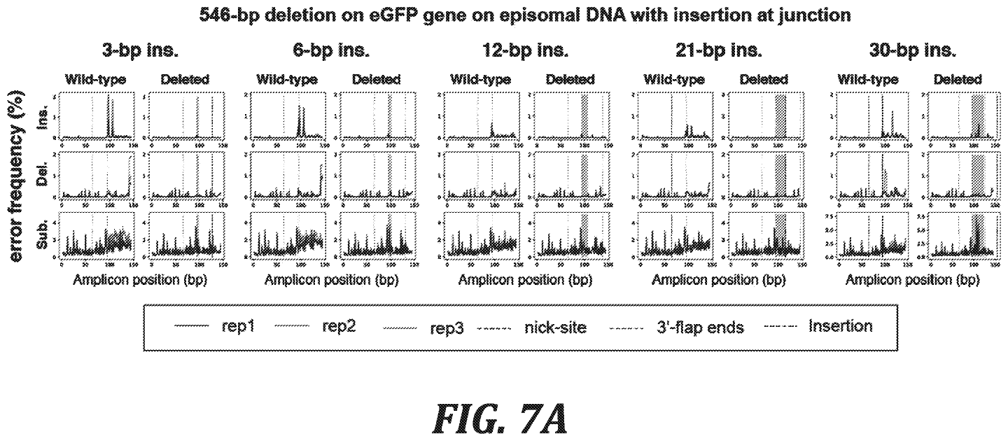

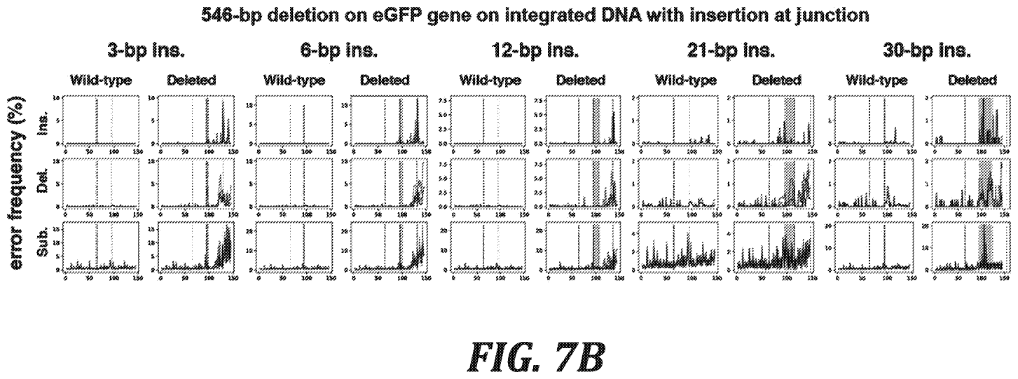

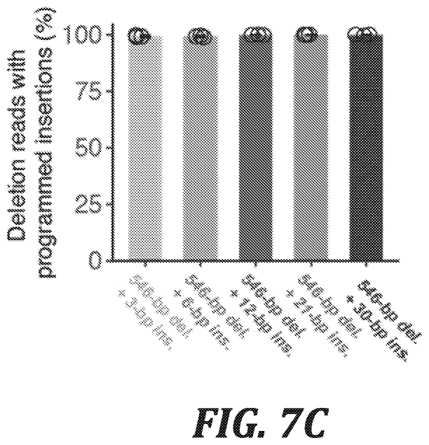

To test this concept, pegRNA pairs were designed that encoded five insertions ranging from 3 to 30 bp at the junction of a 546-bp programmed deletion within eGFP (FIG. 2C). While the main objective was to test the effect of insertion length on deletion efficiency, insertion sequences were selected for their importance in molecular biology, considering that the 3-bp insertion sequence generates an in-frame stop codon. The 6-bp insertion sequence includes the start codon with the surrounding Kozak consensus sequence. The 12-bp insertion sequence includes tandem repeats of m6A post-transcriptional modification consensus sequence of GGACAT (Dominissini, D. et al. Topology of the human and mouse m6A RNA methylomes revealed by m6A-seq. Nature 485, 201-206 (2012).). The 21-bp insertion sequence includes T7 RNA polymerase promoter sequence. The 30-bp insertion sequence encodes for the in-frame FLAG-tag peptide sequence when translated. The estimated efficiencies for simultaneous short insertion and deletion within the episomal eGFP gene in HEK293T cells were comparable to the 546-bp deletion alone, ranging from 83% to 90% for the various programmed insertions (FIG. 2D). Also, insertion, deletion and substitution error rates at deletion junctions and across programmed insertions were comparable to the background error frequencies (FIGS. 2E and 7A). As expected, the vast majority (>99%) of reads containing the programmed deletion also contained the insertion (FIG. 2F), indicating that the full lengths of the pair of 3′-DNA flaps generated following the programmed pegRNA sequences specify the repair outcome (FIG. 2A).

PRIME-Del Induces Precise Deletions in Genomic DNA

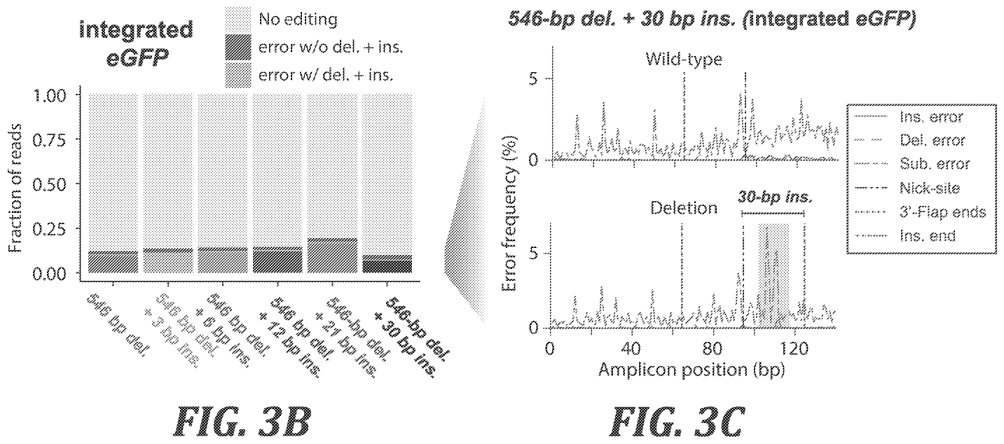

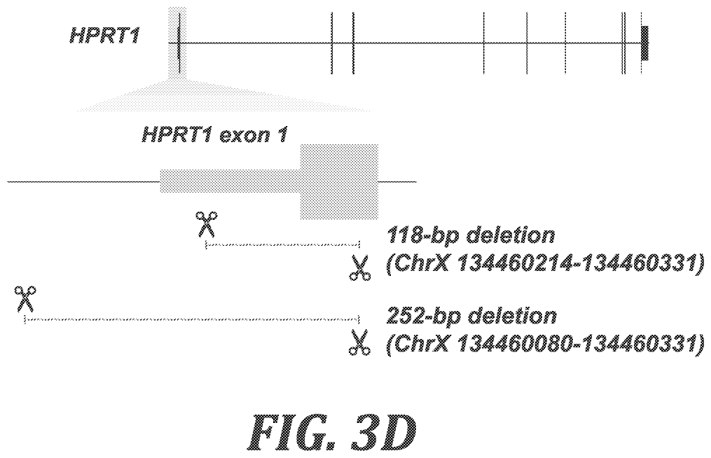

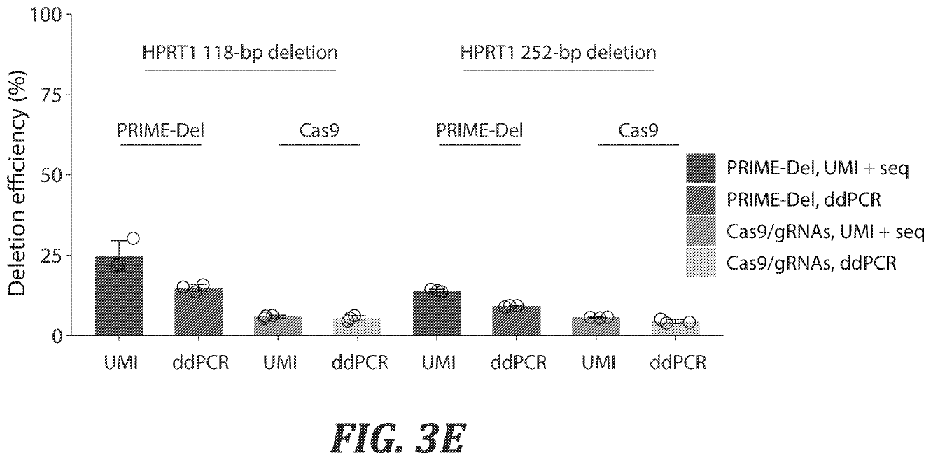

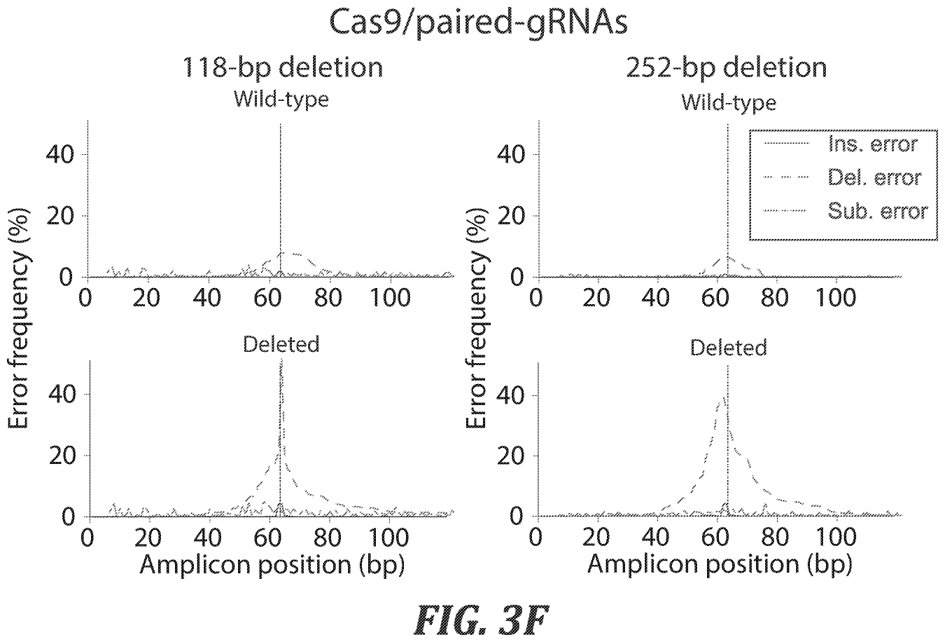

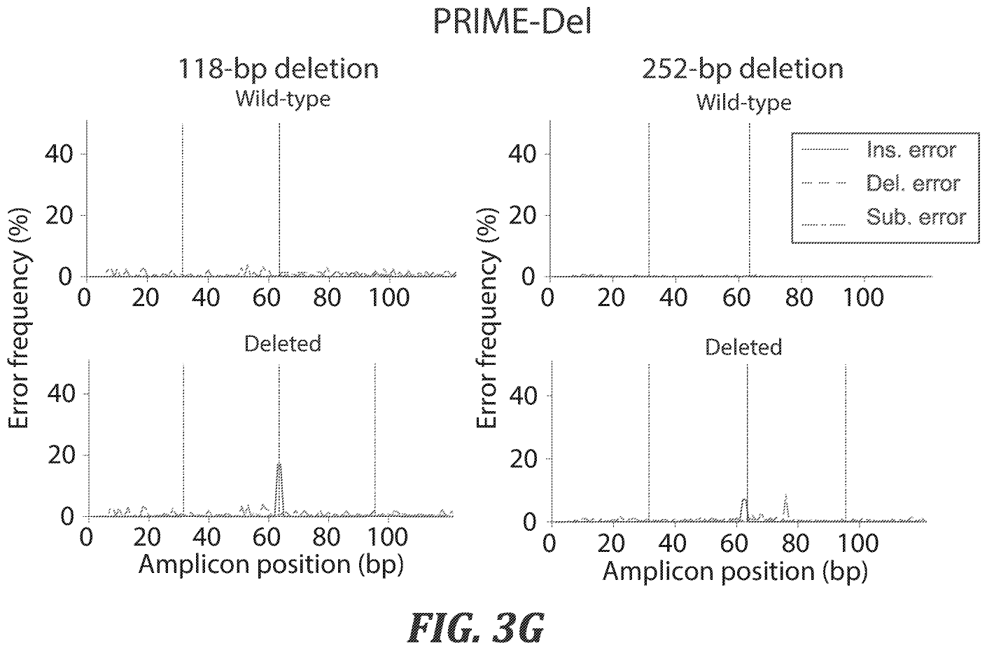

Encouraged by these initial results on editing episomal DNA, PRIME-Del was next tested on a copy of the eGFP gene integrated into the genome. First, the polyclonal HEK293T cells that carry the eGFP gene was generated by lentiviral transduction, followed by flow-sorting to select GFP-positive cells (FIG. 3A). Then the same pairs of pegRNAs encoding concurrent deletion and insertions (546-bp deletion with or without short insertions at the deletion junction) were tested by transfecting pegRNAs and Prime Editor-2 enzyme without eGFP (pCMV-PE2; Addgene #132775) to these cells. Although editing efficiencies decreased substantially in comparison to episomal eGFP (7-17%; FIG. 3B), errors that were clearly associated with editing remained undetectable (FIGS. 3C and 7B). Specifically, there was no consistent pattern of error classes above background level accumulating at the nick-site or 3′-DNA-flap incorporation sites. Also, as previously noted, the vast majority of reads with the 546-bp deletion also contained programmed insertions (FIG. 7C).

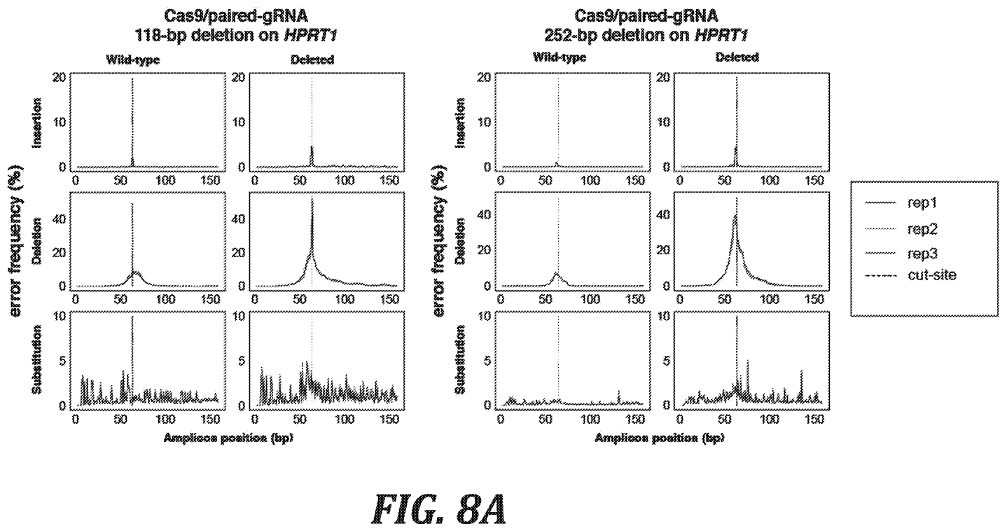

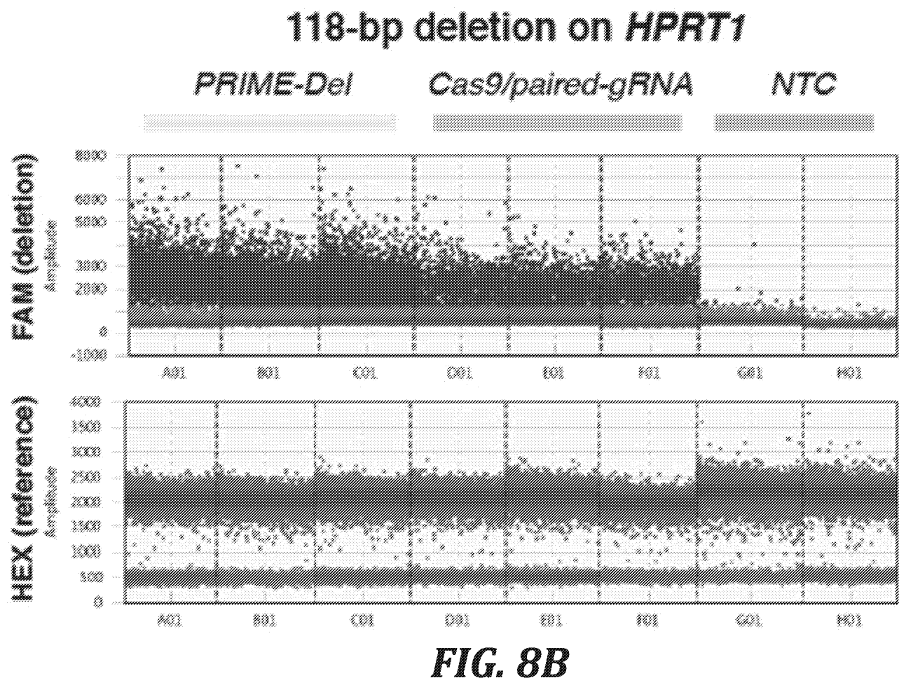

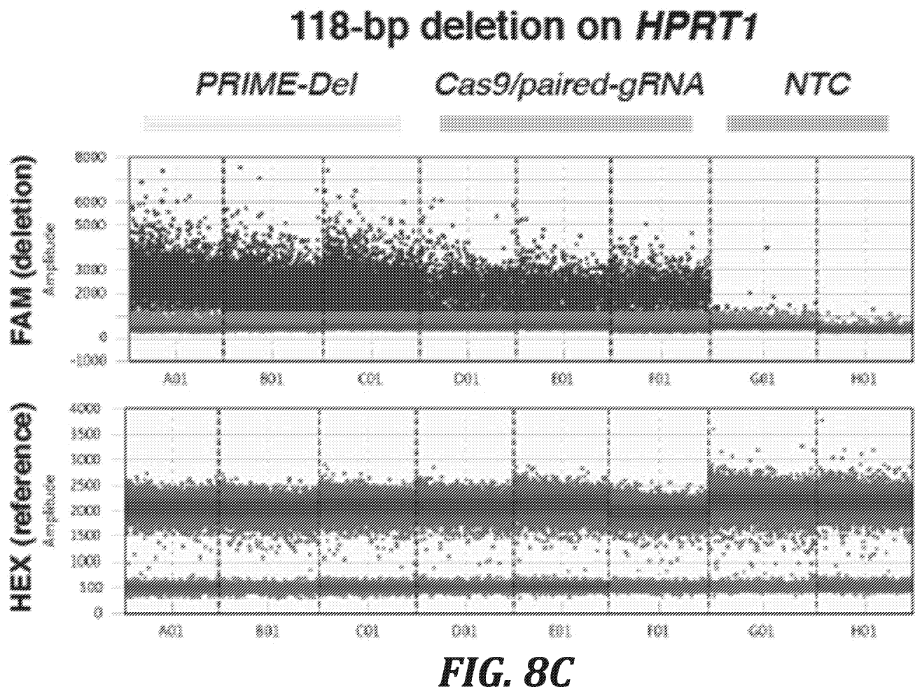

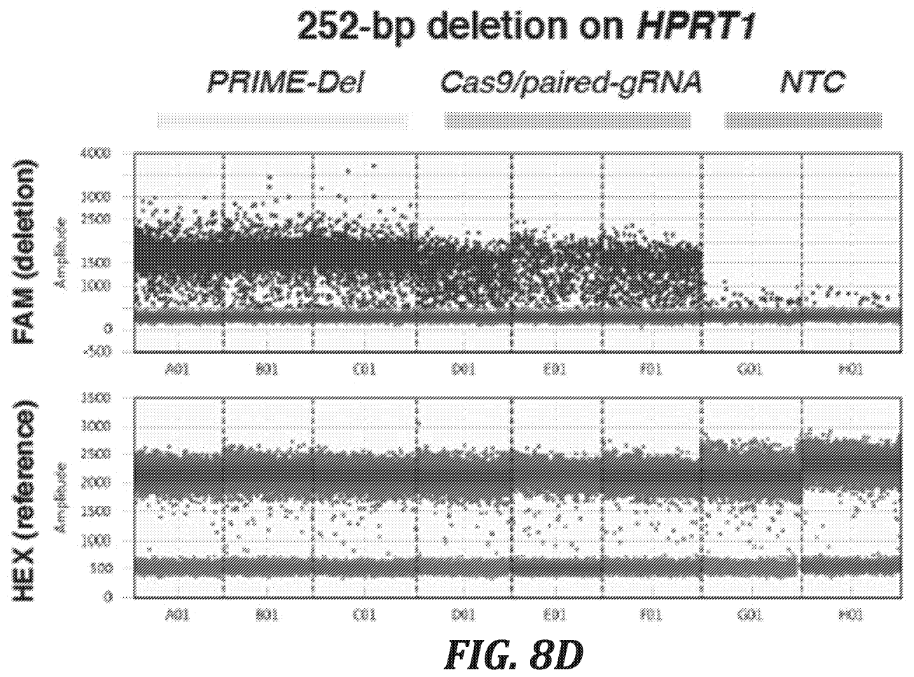

To test PRIME-Del on native genes, two pairs of pegRNAs were designed that respectively specified 118 and 252-bp deletions within exon 1 of HPRT1 (FIG. 3D). Scanning deletion screen across the HPRT1 locus was previously performed a using a Cas9/paired-sgRNA strategy (Gasperini, M. et al. CRISPR/Cas9-Mediated Scanning for Regulatory Elements Required for HPRT1 Expression via Thousands of Large, Programmed Genomic Deletions. Am. J. Hum. Genet. 101, 192-205 (2017)). To directly compare PRIME-Del with Cas9/paired-sgRNAs in programming genomic deletions, the same deletions were attempted with the same guides but substituting Prime Editor-2 enzyme with Cas9 in transfection of HEK293T cells. The resulting deletion efficiencies were quantified using two independent methods: First, the aforedescribed strategy of appending 15-bp unique molecular identifier (UMI) sequence via linear PCR step was used, before the standard PCR and sequencing readout. Resulting sequencing reads are collapsed by shared UMIs to minimize possible biases introduced in the PCR amplification and sequencing cluster generation steps. Second, droplet-digital PCR (ddPCR), which partitions genomic DNA into emulsion droplets before PCR amplification and fluorescence read-out of TaqMan probes within each droplet was used. The probe was designed to bind at the deletion junction, which would generate fluorescence signals specifically in the presence of the deletion. The design of reporter probe aims to quantify the precise editing efficiencies, as errors introduced at the deletion junction are less likely to induce efficient binding of the probe during PCR (Watry, H. L. et al. Rapid, precise quantification of large DNA excisions and inversions by ddPCR. Sci. Rep. 10, 14896 (2020)). Signals from deletions were normalized to the reference signal from detecting the copy-number of RPP30 gene, which has been previously characterized and often used as a standard in ddPCR assay (Watry, H. L. et al., Sci. Rep. 10, 14896 (2020), supra). At exon 1 of HPRT1, comparable deletion efficiencies were observed for the PRIME-Del and Cas9/paired-sgRNA strategies in HEK293T, ranging from 5% to 30% efficiencies for 118-bp and 252-bp deletions (FIG. 3E). Of note, consistently lower efficiencies with the ddPCR assay were observed compared to the UMI-based sequencing assay. While this could be due to overestimation of efficiencies by the UMI-based approach, it is also noted that PCR amplification of the target region may be inefficient in the ddPCR assay based on the lack of clear separation of fluorescence intensities between positive and negative droplets (FIGS. 8C and 8D).

As is well established (see, e.g., Canver, M. C. et al. Characterization of genomic deletion efficiency mediated by clustered regularly interspaced short palindromic repeats (CRISPR)/Cas9 nuclease system in mammalian cells. J. Biol. Chem. 289, 21312-21324 (2014); Byrne, S. M., et al. Multi-kilobase homozygous targeted gene replacement in human induced pluripotent stem cells. Nucleic Acids Res. 43; and Gasperini, M. et al. CRISPR/Cas9-Mediated Scanning for Regulatory Elements Required for HPRT1 Expression via Thousands of Large, Programmed Genomic Deletions. Am. J. Hum. Genet. 101, 192-205 (2017)), the Cas9/paired-sgRNA strategy often resulted in errors (mostly short deletions), whether with or without the intended deletion (FIGS. 3F, 3G, and 8A). Of reads lacking the intended 118-bp or 252-bp deletions, 12% or 12% also contained an unintended indel at the observable target site, respectively (these are underestimates, because they only account for one of two target sites) (FIG. 3F, top). Of reads containing the intended 118-bp or 252-bp deletions, 38% or 34% also contained an unintended indel at the deletion junction, respectively (FIG. 3F, bottom). Such junctional errors are an established consequence of error-prone repair by NHEJ. In contrast, unintended indels were far less common with PRIME-Del (FIGS. 3G and 8B). Of reads lacking the intended 118-bp or 252-bp deletions, 1.1% or 0.5% also contained an unintended short indel at the observable target site, respectively (FIG. 3G, top). Of reads containing the intended 118-bp or 252-bp deletions, 12% or 2.7% also contained an unintended indel at the deletion junction, respectively (FIG. 3G, bottom). The pattern of higher correct editing efficiencies for PRIME-Del over the Cas9/paired-sgRNA strategy is also suggested by the ddPCR measurements, where the PRIME-Del reports a nearly 2-fold higher precisely edited population for both deletions.

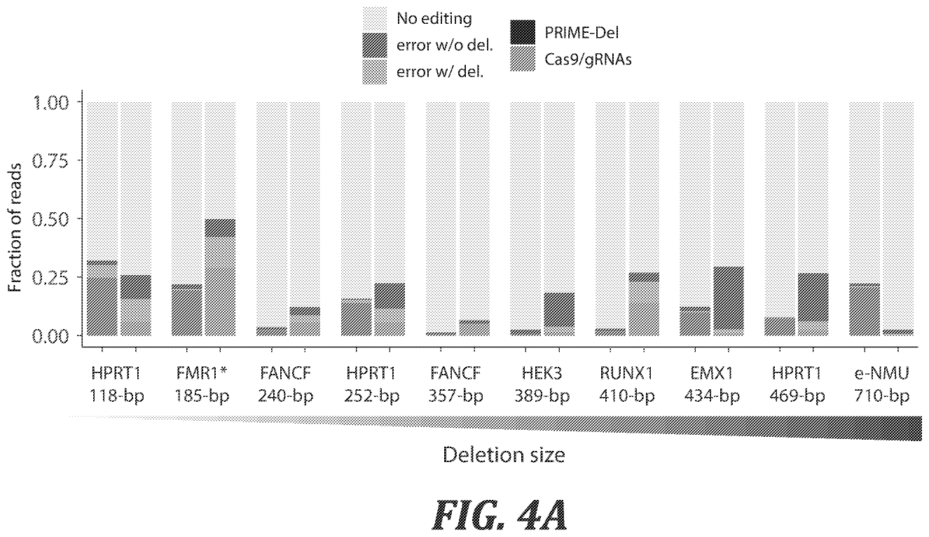

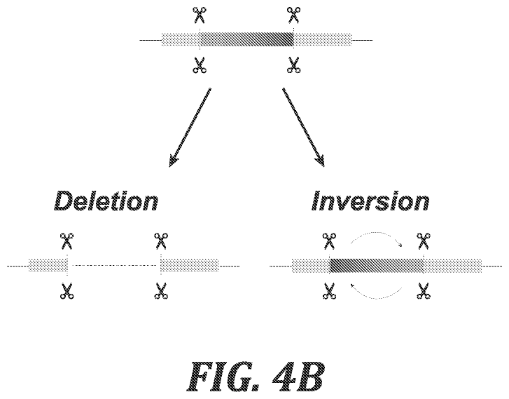

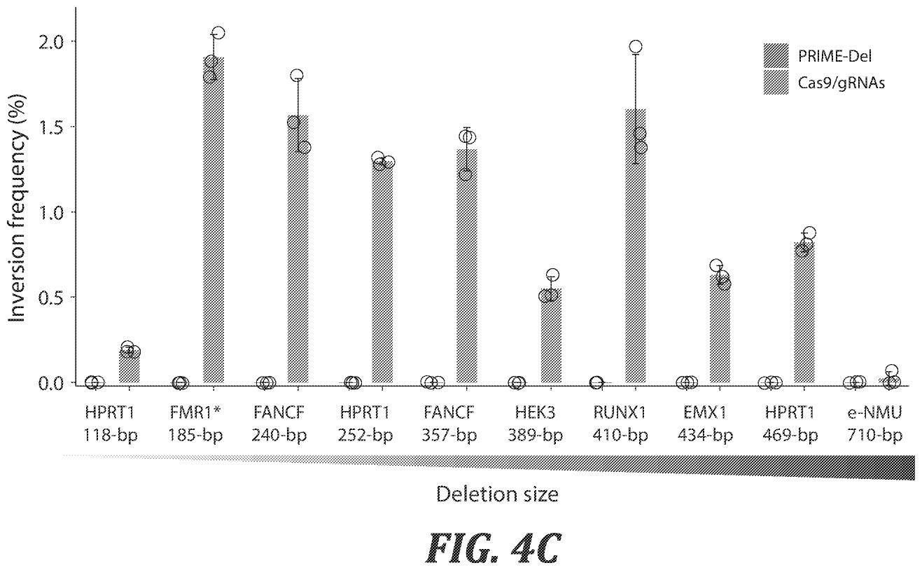

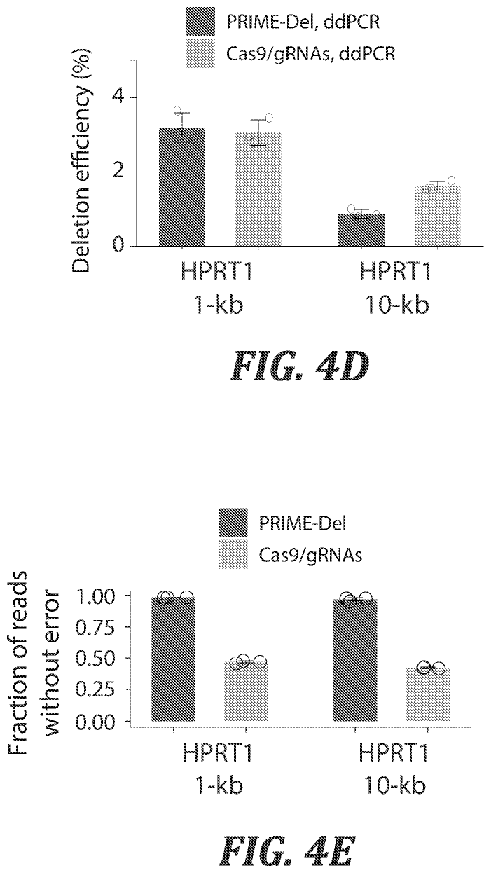

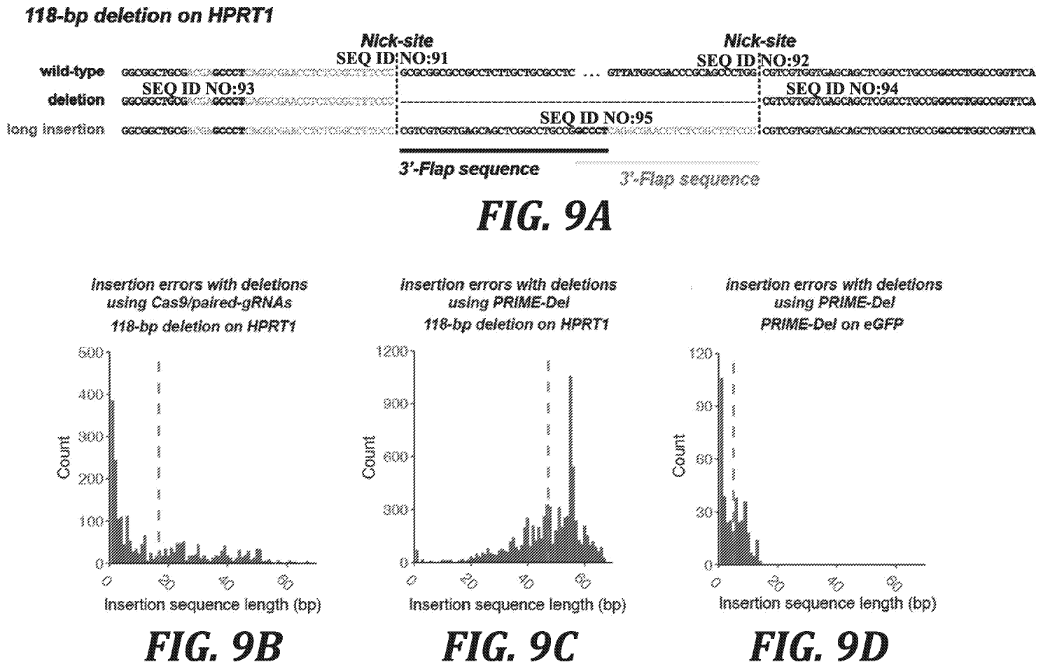

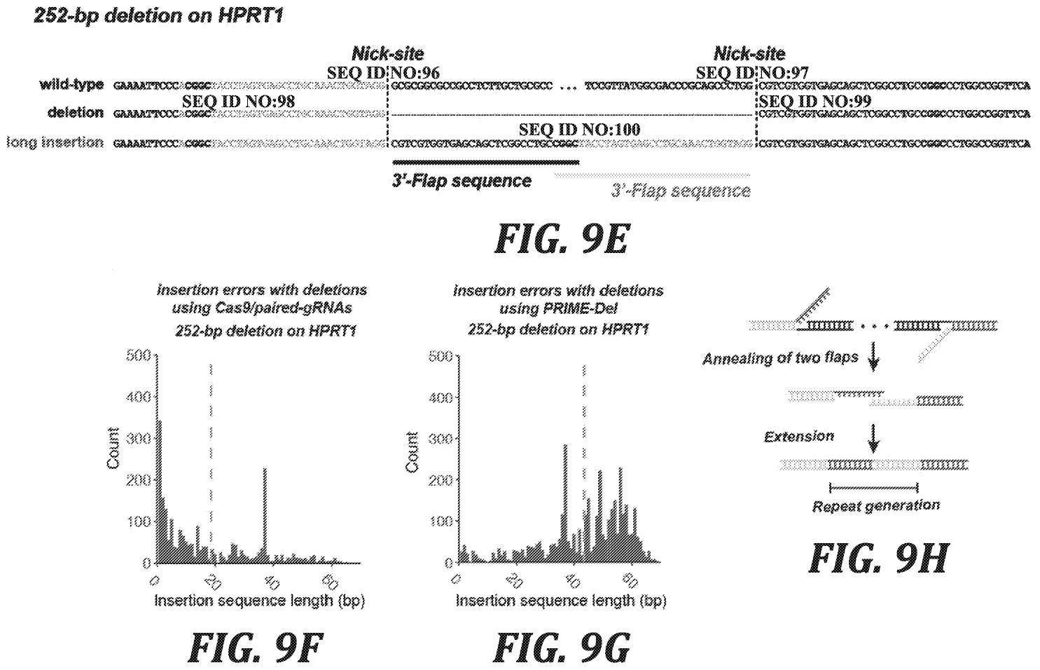

For PRIME-Del, e.g., with the 118-bp deletion on HPRT1, the observation of an appreciable rate of insertions at the deletion junction in association with intended deletions (FIGS. 3G, bottom, and 8B) contrasts with the earlier observations at eGFP, where these rates were consistently equivalent to background. Further investigation of the error mode revealed that these errors corresponded to long insertions (mean 47-bp+/−12-bp; FIGS. 9A-9H). The most frequent long insertion at the 118-bp deletion junction was 55-bp, a chimeric sequence between two 32-bp 3′-DNA flap sequences, overlapping at a ‘GCCCT’ sequence, suggesting its origin from the annealing of GC-rich ends of 3′-DNA flaps. Similar chimeric sequences were observed as insertions at the 252-bp deletion junction, overlapping at ‘GCCG’ within their 3′-DNA flaps. Nonetheless, even with these long insertions, 82% and 91% of all reads containing an indel matched the intended deletion exactly with PRIME-Del, but only 38% and 49% with the Cas9/paired-sgRNA strategies (FIG. 4A). Indel errors from the Cas9/paired-sgRNA strategy are likely underestimated because errors at only one of two Cas9 cut-sites are captured by this sequencing strategy.

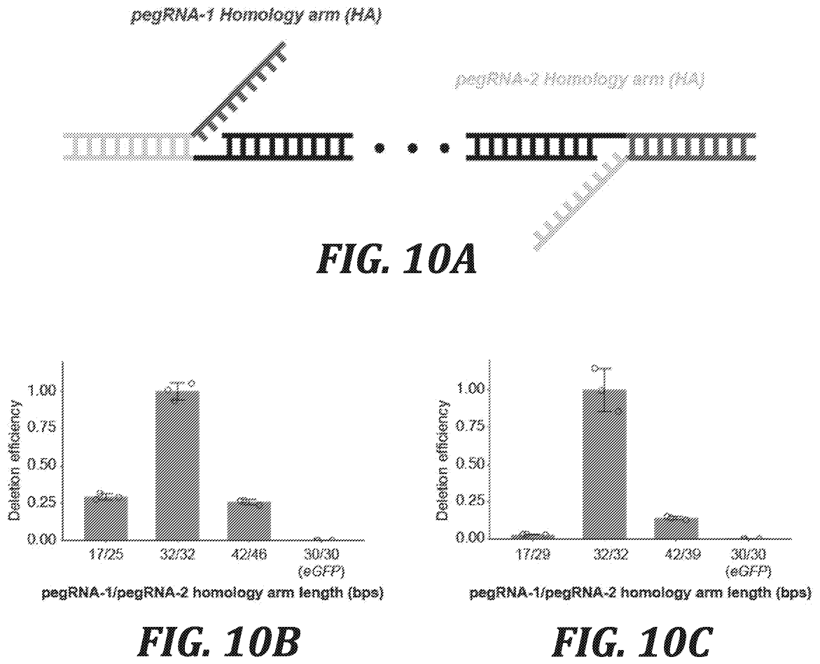

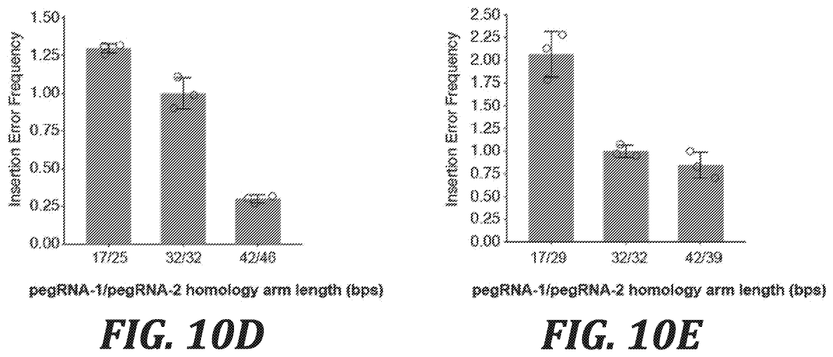

The structure of the observed insertions and the lack of similar errors in applying PRIME-Del to the eGFP locus suggested that this issue might be addressable through alternative pegRNA designs. As one approach, the RT template portion of both pegRNAs was either shortened or lengthened. For 118-bp deletion that used 32-bp RT template lengths for both pegRNAs, homology arms were shortened to either 17- and 25-bp long or lengthened to 42- and 46-bp long (FIG. 10A). Both lengthening and shortening homology arms resulted in decreased deletion efficiencies (29% and 26% of the efficiencies observed with the standard designs for short and long homology arms, respectively) (FIG. 10B). However, among deleted products, lengthening the homology arms also tended to decrease the long-insertion error frequency (to 30% of the standard design), while shortening the homology arms increased the insertion error frequency (to 129% of the standard design) (FIG. 10D). Similar trends were observed with the 252-bp deletion, where shortening or lengthening homology arms decreased the deletion efficiency (FIG. 10C), while lengthening the homology arm increased precision (FIG. 10E). As a further control, substituting the sequence of the RT template to that used for programming a 546-bp deletion at eGFP failed to induce deletions for both 118-bp and 252-bp constructs targeting HPRT1 (FIGS. 10B and 10C), fortifying the conclusion that PRIME-Del deletions are specific to DNA repair guided by the homology arm sequences.

Genomic deletion was further applied using PRIME-Del at additional native loci, altogether testing 10 different deletions at 7 loci (FIG. 4A). All deletions were performed in HEK293T cells, quantified deletion efficiencies and error frequencies using UMI-based sequencing assay, and directly compared PRIME-Del with the Cas9/paired-sgRNA method (i.e. using the same guides but substituting in Cas9). Deletion sizes ranged from 118 bp at HPRT1 exon 1 to 710 bp at e-NMU (enhancer for NMU gene) locus. In all 10 cases, substantially lower error rates were observed with PRIME-Del compared to the Cas9/paired-sgRNA method. In five out of ten cases, it was observed that the precise deletion is more efficient with PRIME-Del compared to the Cas9/paired-sgRNA method, suggesting that higher precision does not compromise the deletion efficiencies in general. A strong relationship between the deletion size and efficiency in this range (118 to 710 bps) was not observed for either method.

Inversion of the sequence between two DSBs is a well-documented phenomenon when using the Cas9/paired-sgRNA method (Canver, M. C. et al. Characterization of genomic deletion efficiency mediated by clustered regularly interspaced short palindromic repeats (CRISPR)/Cas9 nuclease system in mammalian cells. J. Biol. Chem. 289, 21312-21324 (2014); Mandal, P. K. et al. Efficient ablation of genes in human hematopoietic stem and effector cells using CRISPR/Cas9. Cell Stem Cell 15, 643-652 (2014); FIG. 4B). To understand the frequency of inversion events using PRIME-Del, sequencing reads were aligned to a reference that was generated by inverting the sequence between two nick-sites. Across 10 deletions in 7 loci at which PRIME-Del was performed, it was observed that virtually no reads aligned to the inverted reference (FIG. 4C), while for Cas9/paired-sgRNA controls, inversions were detected up in up to 2% of reads (FIG. 4C).

To evaluate the length limits of PRIME-Del, two additional deletions were designed, sized 1,064 bps (1 kb) and 10,204 bps (10 kb) at the HPRT1 locus. Since the sequencing-based assay is not well suited to detect amplicons greater than 1 kb, sequencing was used to quantify error frequencies in the deletion product alone, and ddPCR was used to measure the efficiency of precise deletion, again comparing Prime Editor-2 and Cas9 side-by-side. It was observed that while deletion efficiencies between PRIME-Del and the Cas9/paired-sgRNA method were comparable in HEK293T cells (FIG. 4D), PRIME-Del achieves much higher precision, consistent with the observations while inducing shorter deletions. For the 1-kb deletion, both PRIME-Del and the Cas9/paired-sgRNA method achieved nearly 3% deletion efficiency. For the 10-kb deletion, PRIME-Del and the Cas9/paired-sgRNA method achieved 0.8% and 1.6% deletion efficiency, respectively. Upon sequencing amplicons derived from a PCR specific to the post-deletion junction, 98% and 97% of reads lacked indel errors at the junction with PRIME-Del for the 1-kb and 10-kb deletions, respectively, while only 47% and 42% of reads lacked indel errors with the Cas9/paired-sgRNA strategy (FIG. 4E).

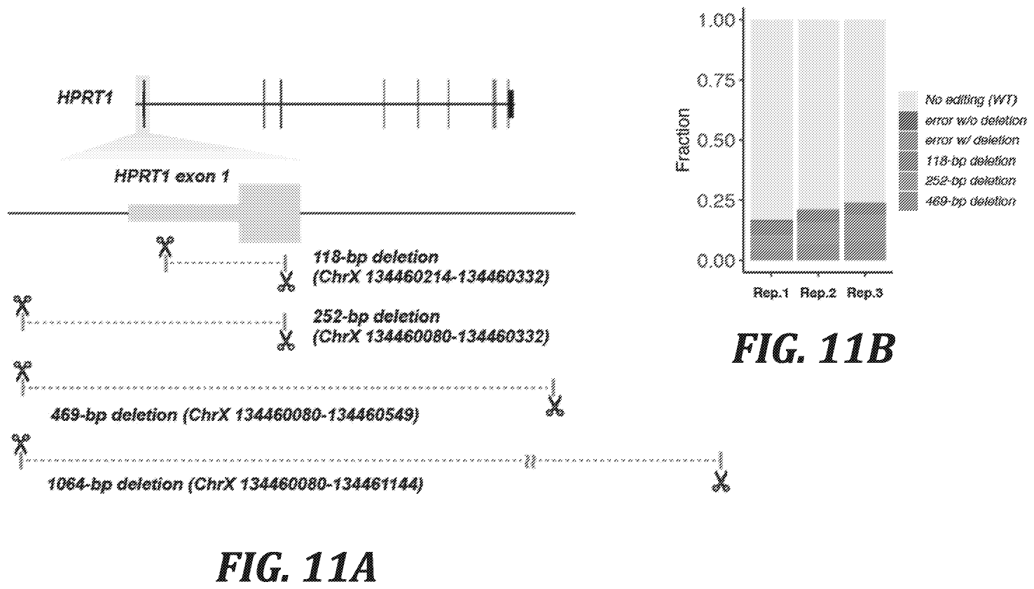

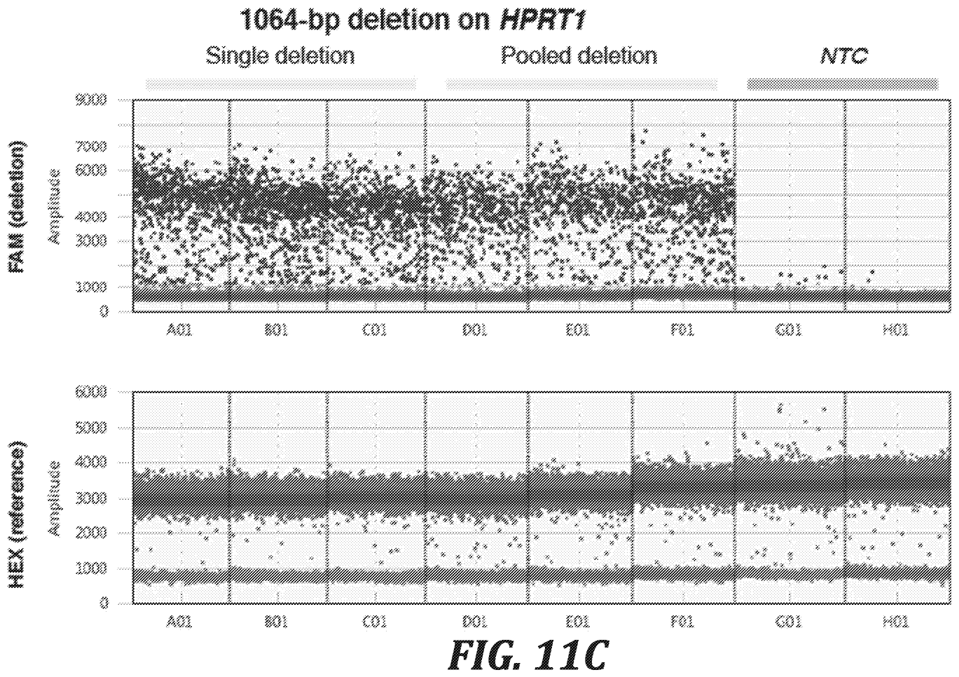

To test whether the PRIME-Del can be “multiplexed”, plasmids encoding paired-pegRNAs programming four different but overlapping deletions (118, 252, 469 and 1064 bps) at the HPRT1 locus were pooled. HEK293T cells were transfected with these plasmids together with a plasmid encoding the Prime Editor-2 enzyme. After incubating cells for 4 days and extracting genomic DNA, sequencing-based quantification was used to estimate 8.5% and 2.8% efficiencies for the 118-, 252-, and 469-bp deletions, and ddPCR was used to estimate 2% efficiency for the 1064-bp deletion (FIGS. 11A-11C). Altogether, it is estimated that 18% of HPRT1 loci carry one of the four programmed deletions, which is comparable to the averaged efficiency of four deletions performed by transfecting a single construct of paired-pegRNA plasmid separately (12%). These results demonstrate that PRIME-Del can be used to concurrently program multiple deletions by using pooled paired-pegRNA constructs similar to Cas9/paired-sgRNA method.

Extending Editing Time Enhances Prime Editing Efficiency

In contrast to Cas9-mediated DSBs followed by NHEJ, both prime editing and PRIME-Del have high editing precision, producing an intended edit or conserving the original editable sequence. It was reasoned that if the editing efficiencies of prime editing and PRIME-Del are limited by the transient availability of PE2/pegRNA molecules in the cell, extending Prime Editor-2 enzyme and pegRNA expression through stable genomic integration or, alternatively, repetitive transfection, would boost the rates of successful editing over time, particularly if uneditable “dead ends” outcomes are not concurrently accruing.

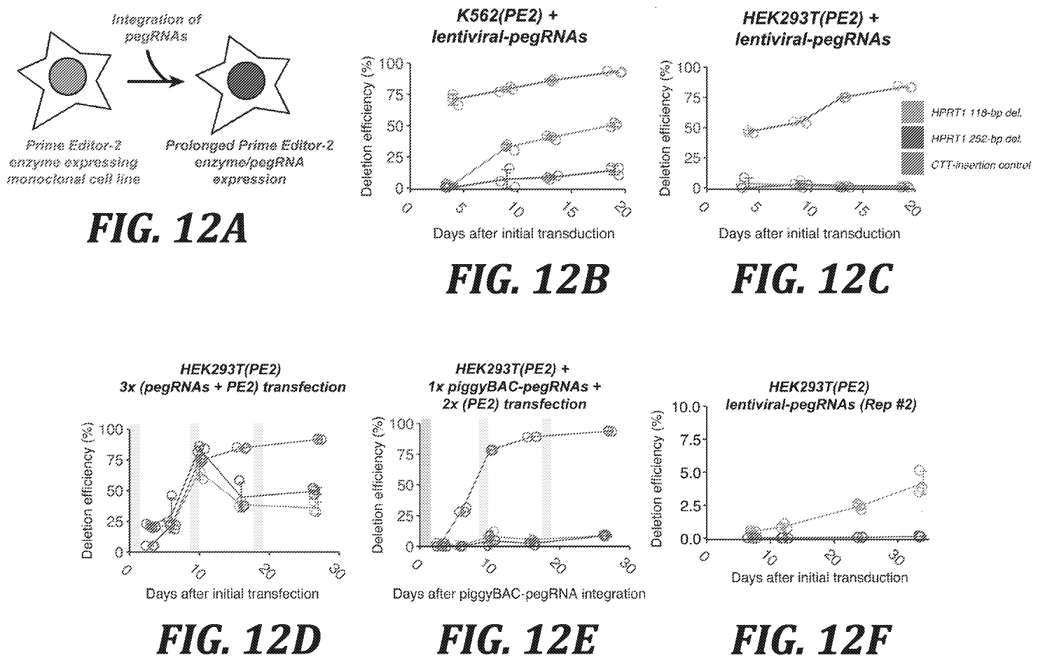

To facilitate prolonged expression, monoclonal HEK293T and K562 cell lines expressing Prime Editor-2 enzyme (termed HEK293T(PE2) and K562(PE2), respectively) were generated and transduced with lentiviral vectors bearing pegRNAs (FIG. 12A). Two different deletions at HPRT1 were tested using PRIME-Del (the aforedescribed 118-bp and 252-bp deletions at exon 1), along with standard prime editing to insert 3-bp (CTT) into the synthetic HEK3 target sequence (Anzalone, A. V. et al. Search-and-replace genome editing without double-strand breaks or donor DNA. Nature 576, 149-157 (2019).). In K562(PE2), a steady increase of the correctly edited population was observed over time, both for CTT-insertion using prime editing and for 118- or 252-bp deletions using PRIME-Del. The end-point prime editing efficiencies for the CTT-insertion were very high, reaching 90% of targets with correct edits by 19 days after the first transduction of pegRNA into K562(PE2) cells (FIG. 12B). The rate of precise deletions using PRIME-Del also reached nearly 50% and 25% for the 118-bp and 252-bp deletions, respectively, by 19 days. In HEK293T(PE2) cells, lower CTT-insertion efficiencies were observed for the first 10 days, but eventually reaching 80-90% by day 19 (FIG. 12C). Unexpectedly, the near-absence of PRIME-Del-induced deletions was observed in HEK293T(PE2) cells (FIG. 12C). While cell-type-specific differences in prime editing cannot be ruled out, the expression levels of Prime Editor-2 enzyme and pegRNAs are suspected to heavily affect the editing efficiency because subsequent attempts in HEK293T(PE2) cells have resulted in accumulating deletions over time (FIGS. 12D and 12F). Together, these results confirm that extended expressions of prime editing or PRIME-Del components can boost efficiency, although it may induce greater off-target effects of prime editing.

Applications of PRIME-Del

This work introduces PRIME-Del, a paired pegRNA strategy for prime editing, and demonstrate that it achieves high precision for programming deletions, both with and without short, programmed insertions. Deletions were tested ranging from 20 to ˜10,000-bp in length at episomal, synthetic genomic, and native genomic loci. The editing efficiency on native genes ranged from 1-30% with a single round of transient transfection in HEK293T cells, although it was also observed that prolonged, high expression of prime editing or PRIME-Del components enhanced editing efficiency in K562 cells. For 12 deletions at seven genomic loci targeted with PRIME-Del, high precision of editing was observed except at HPRT1 exon 1, where long insertions were sometimes observed at the deletion junction (˜5% of total reads). The GC-rich ends of 3′-DNA flap sequences of the pegRNA pairs used at HPRT1 exon 1 appear to underlie the long insertions. Optimizing pegRNA design may be able to eliminate this error mode, and it is shown that lengthening homology arms tends to decrease the frequency of long insertion errors. To facilitate avoidance of this particular error mode, an accompanying Python-based webtool was developed for designing PRIME-Del paired-pegRNA sequences, which notifies the user if such sequences are present in designed pegRNA pairs.

However, even with these insertion errors, PRIME-Del consistently demonstrated higher precision than the Cas9/paired-sgRNA strategy, i.e. for all 12 genomic deletions tested here, PRIME-Del resulted in fewer erroneous outcomes. For these same 12 cases, PRIME-Del exhibited markedly higher precise-deletion efficiencies for five (greater than a factor of two), comparable efficiencies for five (within a factor of two), and markedly lower efficiencies for two (less than half), compared to the Cas9/paired-sgRNA method. Overall, these observations support the view that PRIME-Del achieves higher precision than the Cas9/paired-sgRNA method without compromising editing efficiency.

A potential design-related limitation of PRIME-Del is that relative to the conventional Cas9/paired-sgRNA strategy, it constrains the useable pairs of genomic protospacers, as they need to occur on opposing strands with the PAM sequences oriented towards one another (FIG. 1C). However, the development and optimization of a near-PAMless (Walton, R. T., et al. Unconstrained genome targeting with near-PAMless engineered CRISPR-Cas9 variants. Science 368, 290-296 (2020)) prime editing enzyme (Kweon, J. et al. Engineered prime editors with PAM flexibility. Mol. Ther. (2021) doi:10.1016/j.ymthe.2021.02.022) would relax this constraint. A further limitation is that because of their longer length, cloning a pair of pegRNAs in tandem is more challenging than cloning sgRNA pairs. Each pegRNA used here is 135 to 140 bp in length, such that synthesizing their unique components in tandem as a single, long oligonucleotide approaches the limits of conventional DNA synthesis technology, particularly for goals requiring array-based synthesis of paired pegRNA libraries.

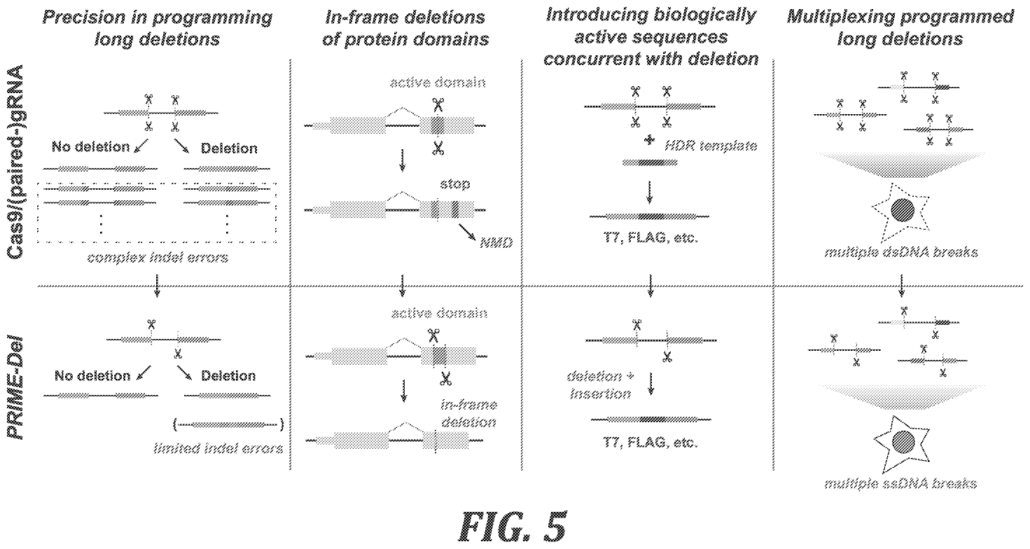

Notwithstanding these limitations, PRIME-Del offers significant advantages over alternatives across several potential areas of application (FIG. 5). Most straightforwardly, PRIME-Del can be used for precise programming of deletions up to at least 10 kb; there are no indications yet establishing an upper limit. In addition to the much lower indel error rate observed at the deletion junction compared to the Cas9/paired-sgRNA strategy, inducing paired nicks is less likely to result in large, unintended deletions locally, rearrangements genome-wide (chromothripsis; see Leibowitz, M. L. et al. Chromothripsis as an on-target consequence of CRISPR—Cas9 genome editing. Nature Genetics (2021) doi:10.1038/s41588-021-00838-7), or off-target editing (Kosicki, M., et al. Repair of double-strand breaks induced by CRISPR-Cas9 leads to large deletions and complex rearrangements. Nat. Biotechnol. 36, 765-771 (2018), Anzalone, A. V. et al. Search-and-replace genome editing without double-strand breaks or donor DNA. Nature 576, 149-157 (2019), Schene, I. F. et al. Nature communications 11.1 (2020): 1-8, Owens, D. D. G. et al. Microhomologies are prevalent at Cas9-induced larger deletions. Nucleic Acids Res. 47, 7402-7417 (2019), and Kim, D. Y. et al. Unbiased investigation of specificities of prime editing systems in human cells. Nucleic Acids Research (2020) doi:10.1093/nar/gkaa764). These characteristics are advantageous for developing therapeutic approaches, e.g. where the PRIME-Del deletes pathogenic regions such as CGG-repeat expansions in 5′-UTR of FMR1, without undesired perturbation of nearby or distant sequences (Khosravi, M. A. et al. Targeted deletion of BCL11A gene by CRISPR-Cas9 system for fetal hemoglobin reactivation: A promising approach for gene therapy of beta thalassemia disease. Eur. J. Pharmacol. 854, 398-405 (2019), Dastidar, S. et al. Efficient CRISPR/Cas9-mediated editing of trinucleotide repeat expansion in myotonic dystrophy patient-derived iPS and myogenic cells. Nucleic Acids Res. 46, 8275-8298 (2018)).

PRIME-Del also allows simultaneous insertion of short sequences at the programmed deletion junction without substantially compromising its efficiency or precision. Inserting short sequences allows for precise deletions of protein domains while preserving the native reading frame, i.e. avoiding a premature stop codon that might otherwise elicit a complex nonsense-mediated decay (NMD) response (El-Brolosy, M. A. et al. Genetic compensation triggered by mutant mRNA degradation. Nature 568, 193-197 (2019), Ma, Z. et al. PTC-bearing mRNA elicits a genetic compensation response via Upf3a and COMPASS components. Nature 568, 259-263 (2019)). Furthermore, inserting biologically active sequences upon deletion is likely to be advantageous in coupling PRIME-Del with technologies, i.e. by inserting epitope tags or T7 promoter sequences that can be used as molecular handles within edited genomic loci.

Additionally, less toxicity via DNA damage by prime editing-based PRIME-Del is expected compared with the conventional Cas9/paired-sgRNA strategy, which may facilitate multiplexing of programmed genomic deletions for frameworks such as scanDel and crisprQTL (Gasperini, M. et al. CRISPR/Cas9-Mediated Scanning for Regulatory Elements Required for HPRT1 Expression via Thousands of Large, Programmed Genomic Deletions. Am. J. Hum. Genet. 101, 192-205 (2017), Gasperini, M. et al. A Genome-wide Framework for Mapping Gene Regulation via Cellular Genetic Screens. Cell 176, 1516 (2019)). For studying the non-coding elements in transcription, efficient and precise deletions up to −10 kb complements the current use of deactivated Cas9-tethered KRAB domain for CRISPR-interference (CRISPRi), which cannot control the range of epigenetic modifications around target regions. As such, it is anticipated that PRIME-Del can be broadly applied in massively parallel functional assays to characterize native genetic elements at base-pair resolution.

### Methods

pegRNA/sgRNA Design

For pegRNA/sgRNA design, CRISPOR (Concordet, J.-P. & Haeussler, M. CRISPOR: intuitive guide selection for CRISPR/Cas9 genome editing experiments and screens. Nucleic Acids Res. 46, W242—W245 (2018)) was initially used to select for 20-bp CRISPR-Cas9 spacers within a given region of interest. Spacers annotated as inefficient were avoided, including U6/H1 terminator and GC-rich sequences, and spacers that had higher predicted efficiencies (Doench scores for U6 transcribed sgRNAs (Doench, J. G. et al. Optimized sgRNA design to maximize activity and minimize off-target effects of CRISPR-Cas9. Nat. Biotechnol. 34, 184-191 (2016)) were generally selected. The length of the RT-template portion of a pegRNA was initially set to 30-bp and extended by 1 to 2-bp if it ended in G or C (Kim, Hui Kwon, et al. “Predicting the efficiency of prime editing guide RNAs in human cells.” Nature Biotechnology 39.2, 198-206(2021), Anzalone, A. V. et al. Search-and-replace genome editing without double-strand breaks or donor DNA. Nature 576, 149-157 (2019).).

Web Tool for PRIME-Del Paired-pegRNA Design

To facilitate PRIME-Del paired-pegRNA design, a Python-based web tool was developed that automates the design process. The software takes a FASTA-formatted sequence file as the input, identifies all possible PAM sequences within the provided region, and initially generates all potential paired pegRNA sequences to program deletions. The software can also optionally take as input scored sgRNA files generated using Flashfry (McKenna, A. & Shendure, J. FlashFry: a fast and flexible tool for large-scale CRISPR target design. BMC Biol. 16, 74 (2018))https://paperpile.com/c/gGxRnW/aYplb, CRISPOR or GPP sgRNA designer(Concordet, J.-P. & Haeussler, M. CRISPOR: intuitive guide selection for CRISPR/Cas9 genome editing experiments and screens. Nucleic Acids Res. 46, W242—W245 (2018)); this is highly recommended to identify effective CRISPR-Cas9 spacers. For FlashFry and CRISPOR, sgRNA spacers with MIT specificity scores (Hsu, P. D. et al. DNA targeting specificity of RNA-guided Cas9 nucleases. Nat. Biotechnol. 31, 827-832 (2013)) below 50 are filtered out as recommended by CRISPOR. From initially generated pegRNA pairs, the software selects relevant ones based on additional user-provided design parameters. For example, the user can define the deletion size range. The user can also define the start and end position of desired deletion, and the software will filter to pegRNA pairs present windows centered at those coordinates. pegRNAs for deletions whose junctions do not fall at PAM sites can be designed using the option ‘--precise’ (-p), which adds insertion sequences to both pegRNAs to facilitate the desired edit.

The PRIME-Del design software also enables additional design constraints to be specified. The pegRNA RT-template length (also known as the homology arm) is set to 30-bp by default, unless specified otherwise by the user. The pegRNA PBS length is set to 13-bp from the PE2 nick-site by default, unless specified otherwise by the user. The nick position relative to the PAM sequence is predicted using previously identified parameters (Lindel (Chen, W. et al. Massively parallel profiling and predictive modeling of the outcomes of CRISPR/Cas9-mediated double-strand break repair. Nucleic Acids Research vol. 47, 7989-8003 (2019))), and RT-template length is adjusted accordingly if the predicted likelihood of generating a nick at a non-canonical position is greater than 25%. PegRNA sequences that include RNA polymerase III terminator sequences (more than four consecutive T's) are filtered out. The software generates warning messages if more than 4 out of 5 bp in either 3′-DNA-flap are either G or C. Code is available at gituhub (github.com/shendurelab/Prime-del), and interactive webpage is available at primedel.uc.r.appspot.com/.

pegRNA cloning

After designing pegRNA pairs, the Golden-Gate cloning strategy outlined by Anzalone et al. (Anzalone, A. V. et al. Nature 576, 149-157 (2019)) was followed, assembling three dsDNA fragments and one plasmid backbone. The first dsDNA fragment contains the pegRNA-1 spacer sequence, annealed from two complementary synthetic single-strand DNA oligonucleotides (IDT) with 4-bp 5′-overhangs. The second dsDNA fragment contains the pegRNA-1 sgRNA scaffold sequence, annealed from two DNA oligonucleotides with 5′-end phosphorylation at the end of 4-bp overhang. The third dsDNA fragment contains the pegRNA-1 RT template sequence and primer binding sequence (PBS), pegRNA-1 terminator sequence (six consecutive T's), and pegRNA-2 sequence with H1 promoter sequence. This was generated by appending pegRNA-1 portion and pegRNA-2 portion to two ends of gene fragments (purchased as gBlocks from IDT) by PCR amplification. The gene fragments contained the pegRNA-1 terminator sequence, H1 promoter sequence, pegRNA-2 spacer sequence, and pegRNA-2 sgRNA scaffold sequences. The forward primer included the BsmBI or Bsal restriction site, pegRNA-1 RT template sequence and PBS. The reverse primer included pegRNA-2 RT template, PBS, and BsmBI or Bsal restriction site. PCR fragments (sized between 300 and 400 bp) were purified using 1.0X AMPure (Beckman Coulter) and mixed with two other dsDNA fragments and linearized backbone vector with corresponding overhangs for Golden-Gate-based assembly mix (BsmBI or Bsal golden-gate assembly mix from New England Biolabs). For the pegRNA cloning backbone, either the GG-acceptor plasmid (Addgene #132777) or piggyBAC-cargo vector that carries the blasticidin-resistance gene were used. Each construct plasmid was transformed into Stbl Competent E. coli (NEB C3040H) for amplification and purified using a miniprep kit (Qiagen). Cloning was verified using Sanger sequencing (Genewiz).

Tissue Culture, Transfection, Lentiviral Transduction, and Monoclonal Line Generation

HEK293T and K562 cells were purchased from ATCC. HEK293T cells were cultured in Dulbecco's modified Eagle's medium with high glucose (GIBCO), supplemented with 10% fetal bovine serum (Rocky Mountain Biologicals) and 1% penicillin-streptomycin (GIBCO). K562 cells were cultured in RPMI 1640 with L-Glutamine (Gibco), supplemented with 10% fetal bovine serum (Rocky Mountain Biologicals) and 1% penicillin-streptomycin (GIBCO). HEK293T and K562 cells were grown with 5% CO 2 at 37 C.

For transient transfection, about 50,000 cells were seeded to each well in a 24-well plate and cultured to 70-90% confluency. For prime editing, 375 ng of Prime Editor-2 enzyme plasmid (Addgene #132775) and 125 ng of pegRNA or paired-pegRNA plasmid were mixed and prepared with transfection reagent (Lipofectamine 3000) following the recommended protocol from the vendor. For deletion using Cas9/paired-sgRNA, 375 ng of Cas9 plasmid (Addgene #52962) was used instead of Prime Editor-2 enzyme plasmid. Cells were cultured for four to five days after the initial transfection unless noted otherwise, and its genomic DNA was harvested either using DNeasy Blood and Tissue kit (Qiagen) or following cell lysis and protease protocol from Anzalone et al. (Anzalone, A. V. et al. Nature 576, 149-157 (2019)).

For lentiviral generation, about 300,000 cells were seeded to each well in a 6-well plate and cultured to 70-90% confluency. Lentiviral plasmid was transfected along with the ViraPower lentiviral expression system (ThermoFisher) following the recommended protocol from the vendor. Lentivirus was harvested following the same protocol, concentrated overnight using Peg-it Virus Precipitation Solution (SBI), and used within 1-2 days to transduce either K562 or HEK293T cells without a freeze-thaw cycle.

For transposase integration, 500 ng of cargo plasmid and 100 ng of Super piggyBAC transposase expression vector (SBI) were mixed and prepared with transfection reagent (Lipofectamine 3000) following the recommended protocol from the vendor. Prime Editor-2 enzyme-expressing single-cell clones were generated by integrating PE2 using piggyBAC transposase system, selected by marker (puromycin resistance gene), single-cell sorted into 96-well plates using flow-sort apparatus, cultured for 2-3 weeks until confluency, and screened for PE activity by transfecting CTT-inserting pegRNA alone (Addgene #132778) and sequencing the HEK3-target loci.

DNA Sequencing Library Preparation

To quantify programmed deletion efficiency and possible errors generated by PRIME-Del, the targeted region was amplified from purified DNA (˜200 to 1000 bp in length) using two-step PCR and sequenced using Illumina sequencing platform (NextSeq or MiSeq) (FIG. 6A). Each purified DNA sample contains wild-type and edited DNA molecules, which were amplified together using the same pairs of primers through each PCR reaction. For the PCR-amplification, a pair of primers was designed for each genomic locus (amplicon) where entire amplicon sizes, with or without deletion, were greater than 200 bp to avoid potential problems in PCR-amplification, in purifying of PCR products, and in clustering onto the sequencing flow-cell.

The first PCR reaction (KAPA Robust) included 300 ng of purified genomic DNA or 2 uL of cell lysate, 0.04 to 0.4 uM of forward and reverse primers in a final reaction volume of 50 uL. The first PCR reaction was programmed to be: 1) 3 minutes at 95° C., 2) seconds at 95° C., 3) 10 seconds at 65° C., 4) 45 seconds at 72° C., 25-28 cycles of repeating step 2 through 4, and 5) 1 minute at 72° C. Primers included sequencing adapters to their 3′-ends, appending them to both termini of PCR products that amplified genomic DNA. After the first PCR step, products were assessed on 6% TBE-gel and purified using 1.0X AMPure (Beckman Coulter) and added to the second PCR reaction that appended dual sample indexes and flow cell adapters. The second PCR reaction program was identical to the first PCR program except 5-10 cycles were run. Products were again purified using AMPure and assessed on the TapeStation (Agilent) before denatured for the sequencing run. For long deletions that generate amplicons sized 200 to 300 bp, Miseq sequencing platform was used at low (8 pM) input DNA concentration to minimize the short amplicons replacing the long amplicons during clustering, aiming cluster density of 300-400 k/mm 2. Denatured libraries were sequenced using either Illumina NextSeq or MiSeq instruments following the vendor protocols.

For appending 15-bp unique molecular identifiers (UMI), the first PCR reaction was performed in two-steps: First, genomic DNA was linearly amplified in the presence of 0.04 to 0.4 uM of single forward primer in two PCR cycles using KAPA Robust polymerase. The UMI-appending linear PCR reaction was programmed to be: 1) 3 minutes and 15 seconds at 95° C., 2) 1 minute at 65° C., 3) 2 minutes at 72° C., 5 cycles of repeating step 2 and 3, 4) 15 seconds at 95° C., 5) 1 minute at 65° C., 6) 2 minutes at 72° C., and another cycles of repeating step 5 and 6. This reaction was cleaned up using 1.5X AMPure, and subject to the second PCR with forward and reverse primers. In this case, the forward primer anneals to the upstream of UMI sequence and is not specific to the genomic loci. After PCR amplification, products were cleaned up and added to another PCR reaction that appended dual sample indexes and flow cell adapters, similar to other samples.

Sequencing Data Processing and Analysis

The sequencing layout was designed to cover at least 50-bp away from the deletion junction in each direction (FIG. 6A). In case of the paired-end sequencing, PEAR (Zhang, J., et al. PEAR: a fast and accurate Illumina Paired-End reAd mergeR. Bioinformatics 30, 614-620 (2014)) was used to merge the paired-end reads with default parameters and ‘-e’ flag to disable the empirical base frequencies. When 15-bp UMI was present in the sequencing reads, a custom Python script was used to find all reads that share the same UMI, which were collapsed into a single read with the most frequent sequence. The resulting sequencing reads were aligned to two reference sequences (with or without deletion) generally using the CRISPResso2 software (Clement, K. et al. CRISPResso2 provides accurate and rapid genome editing sequence analysis. Nat. Biotechnol. 37, 224-226 (2019))https://paperpile.com/c/gGxRnW/2BRib. Default alignment parameters were used in CRISPResso2, with the gap-open penalty of −20, the gap-extension penalty of −2, and the gap incentive value of 1 for inserting indels at the cut/nick sites. The minimum homology score for a read alignment was explored between 50 and 95 for different amplicon length. Custom python and R scripts were used to analyze the alignment results from CRISPResso2.

Alignment was done using two reference sequences (wild-type and deletion) of same sequence length, generating two sets of reads with respective reference sequences. Deletion efficiencies were calculated as the fraction of total number of reads aligning to the reference sequence with deletion over the total number of reads aligning to either references. Genome editing has three types of error modes: substitution, insertion, and deletion. Each error frequency was plotted across two reference sequences, highlighting in each such plot the Cas9(H840A) nick-site and the 3′-DNA flap incorporation sites.

Droplet Digital PCR (ddPCR) Assay

ddPCR probes were designed following the recommended parameters by Bio-Rad Laboratories. Pre-mixed reference probes and primers for the RPP 30 gene were purchased from Bio-Rad Laboratories. Probes and PCR primers were purchased from Integrated DNA Technologies (IDT). Probes were modified with FAM on their 5′-ends and included double quenchers (IDT PrimeTime qPCR probes). Probe sequences were specifically designed to cover the deletion junction for detecting precise deletion products (Hsu, P. D. et al. DNA targeting specificity of RNA-guided Cas9 nucleases. Nat. Biotechnol. 31, 827-832 (2013)). For detecting each deletion, a 20X primer mix was prepared composed of 18 uM forward-primer, 18 uM reverse-primer, and 5 uM FAM-labeled probe in 50 mM Tris-HCl buffer (pH 8.0 at room temperature). 25 uL of ddPCR reaction mixes were composed of 12.5 uL of 2X Supermix for Probes (no dUTP) (Bio-Rad Laboratories), 1.25 uL of 20X HEX-modified RPP 30 reference mix (Bio-Rad Laboratories), 1.25 (IL of 20X FAM-modified primer nix, 0.5 uL of cell lysate containing genomic DNA, and 9.5 uL of DNAse-free water. 20 uL of ddPCR reaction mix was added to 70 uL of Droplet generation oil for probes and used QX200 Droplet generator (Bio-Rad. Laboratories) to generate droplets. Droplets were transferred to ddPCR 96-well plates (Bio-Rad Laboratories) and run on 96-well thermocyclers (Eppendorf) with the following program: 1) 10 minutes at 95° C., 2) 30 seconds at 94° C., 3) 1 minute at 50° C., 41 cycles of repeating step 2 and 3, 4) 10 minutes on 98° C., and 5) cooled down to 4° C. before loading to QX200 Droplet reader. Temperature ramps were limited to 1° C. per second on all steps on thermocyclers. QX200 Droplet reader and Bio-Rad QuantaSoft Pro software were used to visualize and analyze ddPCR experiments. The deletion efficiencies were taken from the ratio of FAM+(precise-deletion) over HEX+(RPP 30 reference for genomic DNA loading) events.

### Data Availability

Raw sequencing data have been uploaded on Sequencing Read Archive (SRA) and made available to the public with associated BioProject ID PRJNA692623. Selected plasmids used for programming genomic deletions are available from Addgene (ID 172655, 172656, 172657, and 172658).

### Code Availability

Source code for PRIME-Del is available at github.com/shendurelab/Prime-del. An interactive webpage for designing pegRNAs for PRIME-Del is available at primedel.uc.r.appspot.com/.

### Sequence Tables

TABLE 1
Sequences of pegRNA and gRNA used in experiments.

| pegRNA | | SEQ ID | |
|---|---|---|---|
| name | pegRNA Sequence | NO: | Appears in |
| eGFP-24 bp- | cagggtcagcttgccgtagggttttagagctagaaatagcaagttaaaataaggcta | 1 | FIG. 1E |
| pegRNA-1 | gtccgttatcaacttgaaaaagtggcaccgagtcgGTGCacgtaaacggccac | | |
| | aagttcagcgtgtccgacggcaagctgac | | |
| eGFP-24 bp- | caagttcagcgtgtccggcggttttagagctagaaatagcaagttaaaataaggcta | 2 | FIG. 1E |
| pegRNA-2 | gtccgttatcaacttgaaaaagtggcaccgagtcggtgctgcagatgaacttcagg | | |
| | gtcagcttgccgtcggacacgctgaa | | |
| eGFP-91 bp- | cAtaggtcagggtggtcacggttttagagctagaaatagcaagttaaaataaggct | 3 | FIG. 1E |
| pegRNA-1 | agtccgttatcaacttgaaaaagtggcaccgagtcgGTGCacgtaaacggcca | | |
| | caagttcagcgtgtccggaccaccctgacc | | |
| eGFP-91 bp- | caagttcagcgtgtccggcggttttagagctagaaatagcaagttaaaataaggcta | 4 | FIG. 1E |
| pegRNA-2 | gtccgttatcaacttgaaaaagtggcaccgagtcggtgcaagcactgcacTccAt | | |
| | aggtcagggtggtccggacacgctgaa | | |
| eGFP- | catgtgatcgcgcttctcgtgttttagagctagaaatagcaagttaaaataaggctag | 5 | FIGS. 1E- |
| 546 bp- | tccgttatcaacttgaaaaagtggcaccgagtcgGTGCacgtaaacggccaca | | 1H, 2D |
| pegRNA-1 | agttcagcgtgtccgagaagcgcgatca | | |
| eGFP- | caagttcagcgtgtccggggttttagagctagaaatagcaagttaaaataaggcta | 6 | FIGS. 1E- |
| 546 bp- | gtccgttatcaacttgaaaaagtggcaccgagtcggtgcactccagcaggaccatg | | 1H, 2D |
| pegRNA-2 | tgatcgcgcttctcggacacgctgaa | | |
| eGFP- | catgtgatcgcgcttctcgtgttttagagctagaaatagcaagttaaaataaggctag | 7 | FIGS. 2D, |
| 546 bp-INS- | tccgttatcaacttgaaaaagtggcaccgagtcgGTGCacgtaaacggccaca | | 3B |
| 3 bp- | agttcagcgtgtccgGCTagaagcgcgatca | | |
| pegRNA-1 | | | |
| eGFP- | caagttcagcgtgtccggcggttttagagctagaaatagcaagttaaaataaggcta | 8 | FIGS. 2D, |
| 546 bp-INS- | gtccgttatcaacttgaaaaagtggcaccgagtcggtgcactccagcaggaccatg | | 3B |
| 3 bp- | tgatcgcgcttctAGCcggacacgctgaa | | |
| pegRNA-2 | | | |
| eGFP- | catgtgatcgcgcttctcgtgttttagagctagaaatagcaagttaaaataaggctag | 9 | FIGS. 2D, |
| 546 bp-INS- | tccgttatcaacttgaaaaagtggcaccgagtcgGTGCacgtaaacggccaca | | 3B |
| 6 bp- | agttcagcgtgtccgCCATGGagaagcgcgatca | | |
| pegRNA-1 | | | |
| eGFP- | caagttcagcgtgtccggcggttttagagctagaaatagcaagttaaaataaggcta | 10 | FIGS. 2D, |
| 546 bp-INS- | gtccgttatcaacttgaaaaagtggcaccgagtcggtgcactccagcaggaccatg | | 3B |
| 6 bp- | tgatcgcgcttctCCATGGcggacacgctgaa | | |
| pegRNA-2 | | | |
| eGFP- | catgtgatcgcgcttctcgtgttttagagctagaaatagcaagttaaaataaggctag | 11 | FIGS. 2D, |
| 546 bp-INS- | tccgttatcaacttgaaaaagtggcaccgagtcgGTGCacgtaaacggccaca | | 3B |
| 12 bp- | agttcagcgtgtccgGACATAGGACTAagaagcgcgatca | | |
| pegRNA-1 | | | |
| eGFP- | caagttcagcgtgtccggcggttttagagctagaaatagcaagttaaaataaggcta | 12 | FIGS. 2D, |
| 546 bp-INS- | gtccgttatcaacttgaaaaaGTGGCACCGAGTCGGTGCactccag | | 3B |
| 12 bp- | caggaccatgtgatcgcgcttctTAGTCCTATGTCcggacacgctgaa | | |
| pegRNA-2 | | | |
| eGFP- | catgtgatcgcgcttctcgtgttttagagctagaaatagcaagttaaaataaggctag | 13 | FIGS. 2D, |
| 546 bp-INS- | tccgttatcaacttgaaaaagtggcaccgagtcgGTGCacgtaaacggccaca | | 3B |
| 21 bp- | agttcagcgtgtccgTAATACGACTCACTATAGGGAagaagcg | | |
| pegRNA-1 | cgatca | | |
| eGFP- | caagttcagcgtgtccggcggttttagagctagaaatagcaagttaaaataaggcta | 14 | FIGS. 2D, |
| 546 bp-INS- | gtccgttatcaacttgaaaaaGTGGCACCGAGTCGGTGCactccag | | 3B |
| 21 bp- | caggaccatgtgatcgcgcttctTCCCTATAGTGAGTCGTATTA | | |
| pegRNA-2 | cggacacgctgaa | | |
| eGFP- | catgtgatcgcgcttctcgtgttttagagctagaaatagcaagttaaaataaggctag | 15 | FIGS. 2D, |
| 546 bp-INS- | tccgttatcaacttgaaaaagtggcaccgagtcgGTGCacgtaaacggccaca | | 3B |
| 30 bp- | agttcagcgtgtccgGCGGAGGTGACTACAAAGACGATGA | | |
| pegRNA-1 | CGACAagaagcgcgatca | | |
| eGFP- | caagttcagcgtgtccggcggttttagagctagaaatagcaagttaaaataaggcta | 16 | FIGS. 2D, |
| 546 bp-INS- | gtccgttatcaacttgaaaaaGTGGCACCGAGTCGGTGCactccag | | 3B |
| 30 bp- | caggaccatgtgatcgcgcttctTGTCGTCATCGTCTTTGTAGT | | |
| pegRNA-2 | CACCTCCGCcggacacgctgaa | | |
| HPRT1- | AACCTCTCGGCTTTCCCGCGgttttagagctagaaatagcaagtta | 17 | FIG. 3E- |
| 118 bp- | aaataaggctagtccgttatcaacttgaaaaagtggcaccgagtcgGTGCAG | | 3H4B, 4C |
| pegRNA-1 | GGCCGGCAGGCCGAGCTGCTCACCACGACGGGGA | | |
| | AAGCCGAGA | | |
| HPRT1- | AGCTGCTCACCACGACGCCAgttttagagctagaaatagcaagtt | 18 | FIG. 3E- |
| 118 bp- | aaaataaggctagtccgttatcaacttgaaaaagtggcaccgagtcggtgcACG | | 3H4B, 4C |
| pegRNA-2 | AGCCCTCAGGCGAACCTCTCGGCTTTCCCCGTCGT | | |
| | GGTGAGC | | |
| HPRT1- | GCCTGCAAACTGGTAGGCGCgttttagagctagaaatagcaagtt | 19 | FIG. 3E- |
| 252 bp- | aaaataaggctagtccgttatcaacttgaaaaagtggcaccgagtcgGTGCA | | 3H4B, 4C |
| pegRNA-1 | GGGCCGGCAGGCCGAGCTGCTCACCACGACGCCT | | |
| | ACCAGTTTGC | | |
| HPRT1- | AGCTGCTCACCACGACGCCAgttttagagctagaaatagcaagtt | 20 | FIG. 3E- |
| 252 bp- | aaaataaggctagtccgttatcaacttgaaaaagtggcaccgagtcggtgcACG | | 3H4B, 4C |
| pegRNA-2 | GCTACCTAGTGAGCCTGCAAACTGGTAGGCGTCGT | | |
| | GGTGAGC | | |
| FMR1- | GGTGGAGGGCCGCCTCTGAGgttttagagctagaaatagcaagtt | 21 | FIG. 3H |
| 185 bp- | aaaataaggctagtccgttatcaacttgaaaaagtggcaccgagtcgGTGCA | | |
| pegRNA-1 | GCTCCTCCATCTTCTCTTCAGCCCTGCTAGCAGAG | | |
| | GCGGC | | |
| FMR1- | TCTTCAGCCCTGCTAGCGCCgttttagagctagaaatagcaagtta | 22 | FIG. 3H |
| 185 bp- | aaataaggctagtccgttatcaacttgaaaaaGTGGCACCGAGTCGG | | |
| pegRNA-2 | TGCTTCGGTTTCACTTCCGGTGGAGGGCCGCCTCT | | |
| | GCTAGCAGG | | |
| FANCF- | CAGGACGTCACAGTGACCGAgttttagagctagaaatagcaagt | 23 | FIG. 3H |
| 240 bp- | taaaataaggctagtccgttatcaacttgaaaaagtggcaccgagtcgGTGCT | | |
| pegRNA-1 | TCGCGCACCTCATGGAATCCCTTCTGCAGCGTCAC | | |
| | TGTGACGT | | |
| FANCF- | GGAATCCCTTCTGCAGCACCgttttagagctagaaatagcaagtt | 24 | FIG. 3H |
| 240 bp- | aaaataaggctagtccgttatcaacttgaaaaagtggcaccgagtcggtgcTTC | | |
| pegRNA-2 | TCCAGCAGGCGCAGAGAGAGCAGGACGTCACAGT | | |
| | GACGCTGCAGAAGGGA | | |
| FANCF- | CTCTTGGAGTGTCTCCTCATgttttagagctagaaatagcaagtta | 25 | FIG. 3H |
| 357 bp- | aaataaggctagtccgttatcaacttgaaaaagtggcaccgagtcgGTGCTT | | |
| pegRNA-1 | CGCGCACCTCATGGAATCCCTTCTGCAGCAGGAGA | | |
| | CACTCCA | | |
| FANCF- | GGAATCCCTTCTGCAGCACCgttttagagctagaaatagcaagtt | 26 | FIG. 3H |
| 357 bp- | aaaataaggctagtccgttatcaacttgaaaaagtggcaccgagtcggtgcAAG | | |
| pegRNA-2 | GCGGGCCAGGCTCTCTTGGAGTGTCTCCTGCTGCA | | |
| | GAAGGGA | | |
| HEK3- | GGCCCAGACTGAGCACGTGAgttttagagctagaaatagcaagt | 27 | FIG. 3H |
| 389 bp- | taaaataaggctagtccgttatcaacttgaaaaagtggcaccgagtcgGTGCA | | |
| pegRNA-1 | TATGACCACCCACCTAATTAaaggagggcaagtCGTGCT | | |
| | CAGTCTG | | |
| HEK3- | ATTAaaggagggcaagtgctgttttagagctagaaatagcaagttaaaataagg | 28 | FIG. 3H |
| 389 bp- | ctagtccgttatcaacttgaaaaagtggcaccgagtcggtgcTCAATCCTT | | |
| pegRNA-2 | GGGGCCCAGACTGAGCACGacttgccctcctt | | |
| RUNX1- | GCATTTTCAGGAGGAAGCGAgttttagagctagaaatagcaagtt | 29 | FIG. 3H |
| 410 bp- | aaaataaggctagtccgttatcaacttgaaaaagtggcaccgagtcgGTGCA | | |
| pegRNA-1 | GTTAAGGATAACTCAGACACAGGCATTCCGGCTTC | | |
| | CTCCTGAAA | | |
| RUNX1- | AGACACAGGCATTCCGGGCAgttttagagctagaaatagcaagt | 30 | FIG. 3H |
| 410 bp- | taaaataaggctagtccgttatcaacttgaaaaagtggcaccgagtcggtgcTTC | | |
| pegRNA-2 | AGAAGAGGGTGCATTTTCAGGAGGAAGCCGGAAT | | |
| | GCCTG | | |
| EMX1- | GAGTCCGAGCAGAAGAAGAAgttttagagctagaaatagcaag | 31 | FIG. 3H |
| 434 bp- | ttaaaataaggctagtccgttatcaacttgaaaaagtggcaccgagtcgGTGCT | | |
| pegRNA-1 | TTATTATTCCCATAGGGAAGGGGGACATTCTTCTG | | |
| | CTCGG | | |
| EMX1- | CATAGGGAAGGGGGACACTGgttttagagctagaaatagcaagt | 32 | FIG. 3H |
| 434 bp- | taaaataaggctagtccgttatcaacttgaaaaagtggcaccgagtcggtgcAG | | |
| pegRNA-2 | GAAGGGCCTGAGTCCGAGCAGAAGAATGTCCCCC | | |
| | TTCC | | |
| HPRT1- | AAGCATGATCAGAACGGTTGgttttagagctagaaatagcaagtt | 33 | FIG. 3H |
| 469 bp- | aaaataaggctagtccgttatcaacttgaaaaagtggcaccgagtcgGTGCAC | | |
| pegRNA-1 | ACGCAGTCCTCTTTTCCCAGGGCTCCCCCGCCTAC | | |
| | CAGTTTGC | | |
| HPRT1- | TTCCCAGGGCTCCCCCGAGGgttttagagctagaaatagcaagtt | 34 | FIG. 3H |
| 469 bp- | aaaataaggctagtccgttatcaacttgaaaaagtggcaccgagtcggtgcACG | | |
| pegRNA-2 | GCTACCTAGTGAGCCTGCAAACTGGTAGGCGGGG | | |
| | GAGCCC | | |
| e-NMU- | aaggggcatgaagtttactggttttagagctagaaatagcaagttaaaataaggcta | 35 | FIG. 3H |
| 710 bp- | gtccgttatcaacttgaaaaagtggcaccgagtcggtgcaggtcagagtcctggct | | |
| pegRNA-1 | ctgtgactcagtgataaacttcatgcc | | |
| e-NMU- | gctctgtgactcagtgacctGGAATAGAAAACAAAAGTTTAA | 36 | FIG. 3H |
| 710 bp- | GTTATTCTAAGGCCAGTCCGGAATCATCCTAAAAA | | |
| pegRNA-2 | GGAGgcaccgagtcgGTGCacatggtacccatgaaggggcatgaagtttat | | |
| | cactgagtcaca | | |
| HPRT1- | AAGCATGATCAGAACGGTTGgttttagagctagaaatagcaagtt | 37 | FIGS. 3I, |
| 1064 bp- | aaaataaggctagtccgttatcaacttgaaaaagtggcaccgagtcgGTGCAC | | 3J |
| pegRNA-1 | GGCTACCTAGTGAGCCTGCAAACTGGTAGGCCGTT | | |
| | CTGATCAT | | |
| HPRT1- | GCCTGCAAACTGGTAGGCGCgttttagagctagaaatagcaagtt | 38 | FIGS. 3I, |
| 1064 bp- | aaaataaggctagtccgttatcaacttgaaaaagtggcaccgagtcggtgcTTT | | 3J |
| pegRNA-2 | GACTATTTTAGCAAGCATGATCAGAACGGCCTACC | | |
| | AGTTTGC | | |
| HPRT1- | AGGTTGGCCCGTAATACCTGgttttagagctagaaatagcaagtt | 39 | FIGS. 3I, |
| 10204 bp- | aaaataaggctagtccgttatcaacttgaaaaagtggcaccgagtcgGTGCAC | | 3J |
| pegRNA-1 | GGCTACCTAGTGAGCCTGCAAACTGGTAGGGTATT | | |
| | ACGGGCCA | | |
| HPRT1- | GCCTGCAAACTGGTAGGCGCgttttagagctagaaatagcaagtt | 40 | FIGS. 3I, |
| 10204 bp- | aaaataaggctagtccgttatcaacttgaaaaagtggcaccgagtcggtgcACT | | 3J |
| pegRNA-2 | TCATGTATTGTCAGGTTGGCCCGTAATACCCTACC | | |
| | AGTTTGC | | |
| HPRT1- | AACCTCTCGGCTTTCCCGCGgttttagagctagaaatagcaagtta | 41 | FIGS. |
| 118 bp- | aaataaggctagtccgttatcaacttgaaaaagtggcaccgagtcgGTGCAG | | 10A-10E |
| HA17 bp- | CTGCTCACCACGACGGGGAAAGCCGAGA | | |
| pegRNA-1 | | | |
| HPRT1- | AGCTGCTCACCACGACGCCAgttttagagctagaaatagcaagtt | 42 | FIGS. |
| 118 bp- | aaaataaggctagtccgttatcaacttgaaaaagtggcaccgagtcggtgcCTC | | 10A-10E |
| HA25 bp- | AGGCGAACCTCTCGGCTTTCCCCGTCGTGGTGAGC | | |
| pegRNA-2 | | | |
| HPRT1- | AACCTCTCGGCTTTCCCGCGgttttagagctagaaatagcaagtta | 43 | FIGS. |
| 118 bp- | aaataaggctagtccgttatcaacttgaaaaagtggcaccgagtcgGTGCTG | | 10A-10E |
| HA42 bp- | AACCGGCCAGGGCCGGCAGGCCGAGCTGCTCACC | | |
| pegRNA-1 | ACGACGGGGAAAGCCGAGA | | |
| HPRT1- | AGCTGCTCACCACGACGCCAgttttagagctagaaatagcaagtt | 44 | FIGS. |
| 118 bp- | aaaataaggctagtccgttatcaacttgaaaaagtggcaccgagtcggtgcTTC | | 10A-10E |
| HA46 bp- | AGGCGGCTGCGACGAGCCCTCAGGCGAACCTCTC | | |
| pegRNA-2 | GGCTTTCCCCGTCGTGGTGAGC | | |
| HPRT1- | GCCTGCAAACTGGTAGGCGCgttttagagctagaaatagcaagtt | 45 | FIGS. |
| HA17 bp- | aaaataaggctagtccgttatcaacttgaaaaagtggcaccgagtcgGTGCA | | 10A-10E |
| pegRNA-1 | GCTGCTCACCACGACGCCTACCAGTTTGC | | |
| HPRT1- | AGCTGCTCACCACGACGCCAgttttagagctagaaatagcaagtt | 46 | FIGS. |
| 252 bp- | aaaataaggctagtccgttatcaacttgaaaaagtggcaccgagtcggtgcGCT | | 10A-10E |
| HA29 bp- | ACCTAGTGAGCCTGCAAACTGGTAGGCGTCGTGGT | | |
| pegRNA-2 | GAGC | | |
| HPRT1- | GCCTGCAAACTGGTAGGCGCgttttagagctagaaatagcaagtt | 47 | FIGS. |
| 252 bp- | aaaataaggctagtccgttatcaacttgaaaaagtggcaccgagtcgGTGCTG | | 10A-10E |
| HA42 bp- | AACCGGCCAGGGCCGGCAGGCCGAGCTGCTCACC | | |
| pegRNA-1 | ACGACGCCTACCAGTTTGC | | |
| HPRT1- | AGCTGCTCACCACGACGCCAgttttagagctagaaatagcaagtt | 48 | FIGS. |
| 252 bp- | aaaataaggctagtccgttatcaacttgaaaaagtggcaccgagtcggtgcAAT | | 10A-10E |
| HA39 bp- | TCCCACGGCTACCTAGTGAGCCTGCAAACTGGTAG | | |
| pegRNA-2 | GCGTCGTGGTGAGC | | |
| HPRT1- | AACCTCTCGGCTTTCCCGCGgttttagagctagaaatagcaagtta | 49 | FIGS. |
| 252 bp- | aaataaggctagtccgttatcaacttgaaaaagtggcaccgagtcgGTGCacgt | | 10A-10E |
| eGFP HA30 | aaacggccacaagttcagcgtgtccgGGGAAAGCCGAGA | | |
| bp-pegRNA- | | | |
| 1 | | | |
| HPRT1- | AGCTGCTCACCACGACGCCAgttttagagctagaaatagcaagtt | 50 | FIGS. |
| 252 bp- | aaaataaggctagtccgttatcaacttgaaaaagtggcaccgagtcggtgcactcc | | 10A-10E |
| eGFP HA30 | agcaggaccatgtgatcgcgcttctCGTCGTGGTGAGC | | |
| bp-pegRNA- | | | |
| 2 | | | |
| HPRT1- | GCCTGCAAACTGGTAGGCGCgttttagagctagaaatagcaagtt | 51 | FIGS. |
| 252 bp- | aaaataaggctagtccgttatcaacttgaaaaagtggcaccgagtcgGTGCacg | | 10A-10E |
| eGFP HA30 | taaacggccacaagttcagcgtgtccgCCTACCAGTTTGC | | |
| bp-pegRNA- | | | |
| 1 | | | |
| HPRT1- | AGCTGCTCACCACGACGCCAgttttagagctagaaatagcaagtt | 52 | FIGS. |
| 252 bp- | aaaataaggctagtccgttatcaacttgaaaaagtggcaccgagtcggtgcactcc | | 10A-10E |
| eGFP HA30 | agcaggaccatgtgatcgcgcttctCGTCGTGGTGAGC | | |
| bp-pegRNA- | | | |
| 2 | | | |

TABLE 2
Sequences of primers used for genomic DNA amplification.

| | | SEQ ID | Appears |
|---|---|---|---|
| primer | pegRNA Sequence | NO: | in |
| eGFP_PCR_ | GCGTCAGATGTGTATAAGAGACAGatgGTGAGCAA | 53 | FIGS. |
| fwd | GGGCGAG | | 1E, 2A-2F |
| eGFP_PCR_ | TTCAGACGTGTGCTCTTCCGATCTAAGATGGTGCG | 54 | FIG. 1E |
| 300_rev | CTCCTG | | |
| eGFP_PCR_ | TTCAGACGTGTGCTCTTCCGATCTACTTGTACAGC | 55 | FIGS. |
| 700_rev | TCGTCCATGCC | | 1E, 2A-2F |
| eGFP_PCR_ | GCGTCAGATGTGTATAAGAGACAGNNNNNNNNNN | 56 | FIG. 1E |
| UMI_fwd | NNNNNatgGTGAGCAAGGGCGAG | | |
| HPRT1_118 | GCGTCAGATGTGTATAAGAGACAGNNNNNNNNNN | 57 | FIGS. |
| bp_fwd | NNNNNGCCTGCTTCTCCTCAGCTTC | | 3E-3H, |
| | | | 4B, 4C |
| HPRT1_118 | TTCAGACGTGTGCTCTTCCGATCTCATTCCCGAAT | 58 | FIGS. |
| bp_rev | CTGCCCTCGG | | 3E-3H, |
| | | | 4B, 4C |
| HPRT1_252 | GCGTCAGATGTGTATAAGAGACAGNNNNNNNNNN | 59 | FIGS. |
| bp_fwd | NNNNNAGCCTCGGCTTCTTCTGGGAG | | 3E-3H, |
| | | | 4B, 4C |
| HPRT1_252 | TTCAGACGTGTGCTCTTCCGATCTCATTCCCGAAT | 60 | FIGS. |
| bp_rev | CTGCCCTCGG | | 3E-3H, |
| | | | 4B, 4C |
| FMR1_185 bp_ | GCGTCAGATGTGTATAAGAGACAGCGCTCAGCTC | 61 | FIG. 3H |
| fwd | CGTTTCGGTTTC | | |
| FMR1_185 bp_ | TTCAGACGTGTGCTCTTCCGATCTATAAGCCATCG | 62 | FIG. 3H |
| rev | CCGTCACTTAG | | |
| FANCF-fwd | GCGTCAGATGTGTATAAGAGACAGNNNNNNNNNN | 63 | FIG. 3H |
| | NNNNNTCCAAGGTGAAAGCGGAAGTAG | | |
| FANCF-rev | CTGAAGGTGATAGCGGTGGCAGATCGGAAGAGCA | 64 | FIG. 3H |
| | CACGTCTGAA | | |
| HEK3- | GCGTCAGATGTGTATAAGAGACAGNNNNNNNNNN | 65 | FIG. 3H |
| 389 bp-fwd | NNNNNGCAAGTAAGCATGCATTTGTAGGCTTGAT | | |
| | G | | |
| HEK3- | TTCAGACGTGTGCTCTTCCGATCTgggttttccagctgttaag | 66 | FIG. 3H |
| 389 bp-rev | cacag | | |
| RUNX1- | GCGTCAGATGTGTATAAGAGACAGNNNNNNNNNN | 67 | FIG. 3H |
| 410 bp-fwd | NNNNNCGCTCCGAAGGTAAAAGAAATCATTGAG | | |
| RUNX1- | TTCAGACGTGTGCTCTTCCGATCTTCTCCTGTACTC | 68 | FIG. 3H |
| 410 bp-rev | TCTGCCTTATAGAGAC | | |
| EMX1- | GCGTCAGATGTGTATAAGAGACAGNNNNNNNNNN | 69 | FIG. 3H |
| 434 bp-fwd | NNNNNGTTCCAGAACCGGAGGACAAAGTAC | | |
| EMX1- | TTCAGACGTGTGCTCTTCCGATCTTGCTGTGGAGC | 70 | FIG. 3H |
| 434 bp-rev | TGGAGGTAGAGAC | | |
| HPRT1- | GCGTCAGATGTGTATAAGAGACAGNNNNNNNNNN | 71 | FIG. 3H |
| 469 bp-fwd | NNNNNAGCCTCGGCTTCTTCTGGGAG | | |
| HPRT1- | TTCAGACGTGTGCTCTTCCGATCTCATTCCCGAAT | 72 | FIG. 3H |
| 469 bp-rev | CTGCCCTCGG | | |
| e-NMU- | GCGTCAGATGTGTATAAGAGACAGNNNNNNNNNN | 73 | FIG. 3H |
| 710 bp-fwd | NNNNNTTGGGTTggtaactggatgttg | | |
| e-NMU- | TTCAGACGTGTGCTCTTCCGATCTgggttttcatgtcctctgctt | 74 | FIG. 3H |
| 710 bp-rev | C | | |
| HPRT1- | GCGTCAGATGTGTATAAGAGACAGNNNNNNNNNN | 75 | FIG. 3J |
| 1064 bp-fwd | NNNNNAGCCTCGGCTTCTTCTGGGAG | | |
| HPRT1- | TTCAGACGTGTGCTCTTCCGATCTCTCTTACAAGC | 76 | FIG. 3J |
| 1064 bp-rev | CAAGTACTGTGCTAAG | | |
| HPRT1- | GCGTCAGATGTGTATAAGAGACAGNNNNNNNNNN | 77 | FIG. 3J |
| 10204 bp- | NNNNNAGCCTCGGCTTCTTCTGGGAG | | |
| fwd | | | |
| HPRT1- | TTCAGACGTGTGCTCTTCCGATCTGAGCATCTCCTT | 78 | FIG. 3J |
| 10204 bp-rev | TTACAACCTAAGC | | |

TABLE 3
Sequences of primers and probes used for droplet digital PCR (ddPCR)
assay. All probes are modified with FAM at 5′-end.

| | | SEQ ID | |
|---|---|---|---|
| primer | pegRNA Sequence | NO: | Appears in |
| HPRT1_118 bp_ | CCTGCTTCTCCTCAGCTTCAG | 79 | FIG. 3E |
| ddPCR_fwd | | | |
| HPRT1_118 bp_ | TTCTCTTCCCACACGCAGTCCTC | 80 | FIG. 3E |
| ddPCR_rev | | | |
| HPRT1_118 bp_ | TCTCGGCTTTCCCCGTCGTGGTGAGC | 81 | FIG. 3E |
| ddPCR_probe | | | |
| HPRT1_252 bp_ | TTCCCACGGCTACCTAGTGAGC | 82 | FIG. 3E |
| ddPCR_fwd | | | |
| HPRT1_252 bp_ | TTCTCTTCCCACACGCAGTCCTC | 83 | FIG. 3E |
| ddPCR_rev | | | |
| HPRT1_252 bp_ | TGCTCACCACGACGCCTACCAGTTTGC | 84 | FIG. 3E |
| ddPCR_probe | | | |
| HPRT1 1064 bp_ | TTCCCACGGCTACCTAGTGAGC | 85 | FIG. 31 |
| ddPCR_fwd | | | |
| HPRT1_1064 bp_ | GAGTTACGGCGGTGATTCCTGC | 86 | FIG. 3I |
| ddPCR_rev | | | |
| HPRT1_1064 bp_ | CTGGTAGGCCGTTCTGATCATGCTTGCT | 87 | FIG. 3I |
| ddPCR_probe | | | |
| HPRT1_10204 | TTCCCACGGCTACCTAGTGAGC | 88 | FIG. 3I |
| bp_ddPCR_fwd | | | |
| HPRT1_10204 | TGCTGTCTTTCAGTCCCCAAAGC | 89 | FIG. 3I |
| bp_ddPCR_rev | | | |
| HPRT1_10204 | CTGGTAGGGTATTACGGGCCAACCTGAC | 90 | FIG. 3I |
| bp_ddPCR_probe | | | |

While illustrative embodiments have been illustrated and described, it will be appreciated that various changes can be made therein without departing from the spirit and scope of the disclosure.

1. A method of editing a double stranded DNA (dsDNA) molecule with a sense strand and antisense strand, comprising:
contacting the dsDNA molecule with a first editing complex specific for a first target sequence on the sense strand of the dsDNA molecule and a second editing complex specific for a second target sequence on the antisense strand of the dsDNA molecule;
wherein the first editing complex and the second editing complex each comprise a fusion editor protein and an extended guide RNA molecule associated therewith, wherein the fusion editors each comprise a functional nickase domain and a functional reverse transcriptase domain;
wherein the extended guide RNA molecule of the first editing complex comprises a first guide domain with a first sequence that hybridizes to the first target sequence and a first extended domain at the 3′ end; and
wherein the extended guide RNA molecule of the second editing complex comprises a second guide domain with a second sequence that hybridizes to the second target sequence and a second extended domain at the 3′ end; and
permitting the functional nickase domain of the first editing complex and the functional nickase domain of the second editing complex to create a first single-stranded break and a second single-stranded break in opposite strands of the dsDNA molecule at the first target sequence and second target sequence, respectively;
permitting the functional reverse transcriptase domain of the first editing complex to generate a first 3′ overhang from the first single-stranded break using the first extended domain as template, and permitting the functional reverse transcriptase domain of the second editing complex to generate a second 3′ overhang from the second single-stranded break using the second extended domain as template;
repairing the dsDNA molecule by excising the portion of the dsDNA originally disposed between the first single-stranded break and second single stranded break and incorporating the first 3′ overhang and second 3′ overhang into the repaired dsDNA molecule.

2. The method of claim 1, wherein the functional nickase domain of the first editing complex and the functional nickase domain of the second editing complex are independently CRISPR-associated (Cas) enzyme, Pyrococcus furiosus Argonaute, and the like, or a functional nickase domain derived therefrom.

3. The method of claim 2, wherein the Cas is Cas9, Cas12, Cas13, Cas3, Cas(I), and the like.

4. The method of claim 1, wherein the functional reverse transcriptase domain of the first editing complex and the functional reverse transcriptase domain of the second editing complex are independently M-MLV RT, HIV RT, group II intron RT (TGIRT), superscript IV, and the like, or a functional domain thereof.

5. The method of claim 1, wherein the first target sequence is disposed in a more 5′ location in the sense strand than the reverse complement of the second target sequence.

6. The method of claim 1, wherein the first target sequence is disposed in a more 3′ location in the sense strand than the reverse complement of the second target sequence.

7. The method of claim 1, wherein the first 3′ overhang and the second 3′ overhang are reverse complements of each other and hybridize in the repairing step.

8. The method of claim 1, wherein the first 3′ overhang comprises a first repair domain with a sequence that corresponds to a sequence immediately 5′ to the second 3′ overhang in the antisense strand, and wherein the second 3′ overhang comprises a second repair domain with a sequence that corresponds to sequence immediately 5′ to the first 3′ overhang in the sense strand.

9. The method of claim 8, wherein the first 3′ overhang further comprises an insertion sequence 5′ to the first repair domain, and wherein the second 3′ overhang comprises a reverse complement sequence of the insertion sequence 5′ to the second repair domain.

10. The method of claim 1, wherein the first 3′ overhang comprises a first repair domain with a sequence that corresponds to a sequence immediately 3′ to the second single stranded break, and wherein the second 3′ overhang comprises a second repair domain with a sequence that corresponds to a sequence immediately 3′ to the first single stranded break, whereby the repairing step results in an inversion of the sequence corresponding to the portion of the dsDNA originally disposed between the first single-stranded break and second single stranded break.

11. The method of claim 1, wherein the first 3′ overhang comprises a first repair domain with a sequence that corresponds to a first end domain of an insertion DNA fragment, wherein the second 3′ overhang comprises a second repair domain with a sequence that corresponds to a second end domain of the insertion DNA fragment, and wherein the first end domain and second end domain are at opposite ends of the insertion DNA fragment or are at distinct sites within a larger dsDNA molecule.

12. The method of claim 1, wherein the portion of the dsDNA molecule originally disposed between the first single-stranded break and second single stranded break that is excised is at least 5 nucleotides long.

13. The method of claim 12, wherein the portion of the dsDNA molecule originally disposed between the first single-stranded break and second single stranded break that is excised is between about 10 nucleotides and 1,000,000 nucleotides long.

14. The method of claim 1, wherein the first editing complex and/or the second editing complex comprise(s) an additional functional domain configured to enhance the efficiency of 3′-overhang generation.

15. The method of claim 1, wherein the fusion editor protein of the first editing complex and/or the second editing complex comprise(s) an additional functional domain configured to enhance the efficiency of DNA repair using generated 3′ overhangs.

16. The method of claim 1, wherein the first guide domain and second guide domain are independently between about 20 and about 200 nucleotides long.

17. The method of claim 16, wherein the first guide domain and second guide domain are independently between about 25 and 100 nucleotides long, between about 25 and 50 nucleotides long, or between about 25 and 40 nucleotides long.

18-26. (canceled)

27. A method of editing one or more double stranded DNA (dsDNA) molecules in a cell, comprising contacting the cell with one or more pairs of first and second editing complexes, or one or more nucleic acids encoding components of the one or more pairs of first and second complexes and permitting the components to be expressed and assembled in the cell;
wherein for each pair of the one or more pairs first and second editing complexes:
the first editing complex is specific for a first target sequence on the sense strand of the dsDNA molecule and the second editing complex specific for a second target sequence on the antisense strand of the dsDNA molecule;
the first editing complex and the second editing complex each comprise a fusion editor protein and an extended guide RNA molecule associated therewith, wherein the fusion editors each comprise a functional nickase domain and a functional reverse transcriptase domain;
the extended guide RNA molecule of the first editing complex comprises a first guide domain with a first sequence that hybridizes to the first target sequence and a first extended domain at the 3′ end; and
the extended guide RNA molecule of the second editing complex comprises a second guide domain with a second sequence that hybridizes to the second target sequence and a second extended domain at the 3′ end; and
for each pair of first and second editing complexes:
permitting the functional nickase domain of the first editing complex and the functional nickase domain of the second editing complex to create a first single-stranded break and a second single-stranded break in opposite strands of the dsDNA molecule at the first target sequence and second target sequence, respectively;
permitting the functional reverse transcriptase domain of the first editing complex to generate a first 3′ overhang from the first single-stranded break using the first extended domain as template, and permitting the functional reverse transcriptase domain of the second editing complex to generate a second 3′ overhang from the second single-stranded break using the second extended domain as template; and
repairing the dsDNA molecule by excising the portion of the dsDNA originally disposed between the first single-stranded break and second single stranded break and incorporating the first 3′ overhang and second 3′ overhang into the repaired dsDNA molecule.

28. The method of claim 27, comprising contacting the cell with a plurality of pairs of first and second editing complexes, or a plurality of nucleic acids encoding components of the plurality of pairs of first and second complexes and permitting the components to be expressed and assembled in the cell, wherein each pair of first and second editing complexes targets different first and second target sequences on the one or more dsDNA molecules in the cell.

29. A kit comprising the first editing complex and the second editing complex as recited in claim 1, wherein the first target sequence on the sense strand and second target sequence on the antisense strand are separated by an intervening sequence, and wherein the first editing complex and the second editing complex are configured to delete intervening sequence, to invert the intervening sequence, and/or inserting one or more new sequences at the first and/or second single stranded breaks induced by the first editing complex and the second editing complex in the target dsDNA molecule.


SEQUENCE LISTING (100 sequences)

SEQ ID NO: 1 - DNA, 139 bp, Artificial sequence
  Synthetic
cagggtcagcttgccgtagggttttagagctagaaatagcaagttaaaataaggctagtccgttatcaacttgaaaaagtggcaccgagtcggtgcacgtaaacggccacaagttcagcgtgtccgacggcaagctgac

SEQ ID NO: 2 - DNA, 139 bp, Artificial sequence
  Synthetic
caagttcagcgtgtccggcggttttagagctagaaatagcaagttaaaataaggctagtccgttatcaacttgaaaaagtggcaccgagtcggtgctgcagatgaacttcagggtcagcttgccgtcggacacgctgaa

SEQ ID NO: 3 - DNA, 139 bp, Artificial sequence
  Synthetic
cataggtcagggtggtcacggttttagagctagaaatagcaagttaaaataaggctagtccgttatcaacttgaaaaagtggcaccgagtcggtgcacgtaaacggccacaagttcagcgtgtccggaccaccctgacc

SEQ ID NO: 4 - DNA, 139 bp, Artificial sequence
  Synthetic
caagttcagcgtgtccggcggttttagagctagaaatagcaagttaaaataaggctagtccgttatcaacttgaaaaagtggcaccgagtcggtgcaagcactgcactccataggtcagggtggtccggacacgctgaa

SEQ ID NO: 5 - DNA, 139 bp, Artificial sequence
  Synthetic
catgtgatcgcgcttctcgtgttttagagctagaaatagcaagttaaaataaggctagtccgttatcaacttgaaaaagtggcaccgagtcggtgcacgtaaacggccacaagttcagcgtgtccgagaagcgcgatca

SEQ ID NO: 6 - DNA, 139 bp, Artificial sequence
  Synthetic
caagttcagcgtgtccggcggttttagagctagaaatagcaagttaaaataaggctagtccgttatcaacttgaaaaagtggcaccgagtcggtgcactccagcaggaccatgtgatcgcgcttctcggacacgctgaa

SEQ ID NO: 7 - DNA, 142 bp, Artificial sequence
  Synthetic
catgtgatcgcgcttctcgtgttttagagctagaaatagcaagttaaaataaggctagtccgttatcaacttgaaaaagtggcaccgagtcggtgcacgtaaacggccacaagttcagcgtgtccggctagaagcgcgatca

SEQ ID NO: 8 - DNA, 142 bp, Artificial sequence
  Synthetic
caagttcagcgtgtccggcggttttagagctagaaatagcaagttaaaataaggctagtccgttatcaacttgaaaaagtggcaccgagtcggtgcactccagcaggaccatgtgatcgcgcttctagccggacacgctgaa

SEQ ID NO: 9 - DNA, 145 bp, Artificial sequence
  Synthetic
catgtgatcgcgcttctcgtgttttagagctagaaatagcaagttaaaataaggctagtccgttatcaacttgaaaaagtggcaccgagtcggtgcacgtaaacggccacaagttcagcgtgtccgccatggagaagcgcgatca

SEQ ID NO: 10 - DNA, 145 bp, Artificial sequence
  Synthetic
caagttcagcgtgtccggcggttttagagctagaaatagcaagttaaaataaggctagtccgttatcaacttgaaaaagtggcaccgagtcggtgcactccagcaggaccatgtgatcgcgcttctccatggcggacacgctgaa

SEQ ID NO: 11 - DNA, 151 bp, Artificial sequence
  Synthetic
catgtgatcgcgcttctcgtgttttagagctagaaatagcaagttaaaataaggctagtccgttatcaacttgaaaaagtggcaccgagtcggtgcacgtaaacggccacaagttcagcgtgtccggacataggactaagaagcgcgatca

SEQ ID NO: 12 - DNA, 151 bp, Artificial sequence
  Synthetic
caagttcagcgtgtccggcggttttagagctagaaatagcaagttaaaataaggctagtccgttatcaacttgaaaaagtggcaccgagtcggtgcactccagcaggaccatgtgatcgcgcttcttagtcctatgtccggacacgctgaa

SEQ ID NO: 13 - DNA, 160 bp, Artificial sequence
  Synthetic
catgtgatcgcgcttctcgtgttttagagctagaaatagcaagttaaaataaggctagtccgttatcaacttgaaaaagtggcaccgagtcggtgcacgtaaacggccacaagttcagcgtgtccgtaatacgactcactatagggaagaagcgcgatca

SEQ ID NO: 14 - DNA, 160 bp, Artificial sequence
  Synthetic
caagttcagcgtgtccggcggttttagagctagaaatagcaagttaaaataaggctagtccgttatcaacttgaaaaagtggcaccgagtcggtgcactccagcaggaccatgtgatcgcgcttcttccctatagtgagtcgtattacggacacgctgaa

SEQ ID NO: 15 - DNA, 169 bp, Artificial sequence
  Synthetic
catgtgatcgcgcttctcgtgttttagagctagaaatagcaagttaaaataaggctagtccgttatcaacttgaaaaagtggcaccgagtcggtgcacgtaaacggccacaagttcagcgtgtccggcggaggtgactacaaagacgatgacgacaagaagcgcgatca

SEQ ID NO: 16 - DNA, 169 bp, Artificial sequence
  Synthetic
caagttcagcgtgtccggcggttttagagctagaaatagcaagttaaaataaggctagtccgttatcaacttgaaaaagtggcaccgagtcggtgcactccagcaggaccatgtgatcgcgcttcttgtcgtcatcgtctttgtagtcacctccgccggacacgctgaa

SEQ ID NO: 17 - DNA, 141 bp, Artificial sequence
  Synthetic
aacctctcggctttcccgcggttttagagctagaaatagcaagttaaaataaggctagtccgttatcaacttgaaaaagtggcaccgagtcggtgcagggccggcaggccgagctgctcaccacgacggggaaagccgaga

SEQ ID NO: 18 - DNA, 141 bp, Artificial sequence
  Synthetic
agctgctcaccacgacgccagttttagagctagaaatagcaagttaaaataaggctagtccgttatcaacttgaaaaagtggcaccgagtcggtgcacgagccctcaggcgaacctctcggctttccccgtcgtggtgagc

SEQ ID NO: 19 - DNA, 141 bp, Artificial sequence
  Synthetic
gcctgcaaactggtaggcgcgttttagagctagaaatagcaagttaaaataaggctagtccgttatcaacttgaaaaagtggcaccgagtcggtgcagggccggcaggccgagctgctcaccacgacgcctaccagtttgc

SEQ ID NO: 20 - DNA, 141 bp, Artificial sequence
  Synthetic
agctgctcaccacgacgccagttttagagctagaaatagcaagttaaaataaggctagtccgttatcaacttgaaaaagtggcaccgagtcggtgcacggctacctagtgagcctgcaaactggtaggcgtcgtggtgagc

SEQ ID NO: 21 - DNA, 137 bp, Artificial sequence
  Synthetic
ggtggagggccgcctctgaggttttagagctagaaatagcaagttaaaataaggctagtccgttatcaacttgaaaaagtggcaccgagtcggtgcagctcctccatcttctcttcagccctgctagcagaggcggc

SEQ ID NO: 22 - DNA, 137 bp, Artificial sequence
  Synthetic
tcttcagccctgctagcgccgttttagagctagaaatagcaagttaaaataaggctagtccgttatcaacttgaaaaagtggcaccgagtcggtgcttcggtttcacttccggtggagggccgcctctgctagcagg

SEQ ID NO: 23 - DNA, 140 bp, Artificial sequence
  Synthetic
caggacgtcacagtgaccgagttttagagctagaaatagcaagttaaaataaggctagtccgttatcaacttgaaaaagtggcaccgagtcggtgcttcgcgcacctcatggaatcccttctgcagcgtcactgtgacgt

SEQ ID NO: 24 - DNA, 149 bp, Artificial sequence
  Synthetic
ggaatcccttctgcagcaccgttttagagctagaaatagcaagttaaaataaggctagtccgttatcaacttgaaaaagtggcaccgagtcggtgcttctccagcaggcgcagagagagcaggacgtcacagtgacgctgcagaaggga

SEQ ID NO: 25 - DNA, 140 bp, Artificial sequence
  Synthetic
ctcttggagtgtctcctcatgttttagagctagaaatagcaagttaaaataaggctagtccgttatcaacttgaaaaagtggcaccgagtcggtgcttcgcgcacctcatggaatcccttctgcagcaggagacactcca

SEQ ID NO: 26 - DNA, 141 bp, Artificial sequence
  Synthetic
ggaatcccttctgcagcaccgttttagagctagaaatagcaagttaaaataaggctagtccgttatcaacttgaaaaagtggcaccgagtcggtgcaaggcgggccaggctctcttggagtgtctcctgctgcagaaggga

SEQ ID NO: 27 - DNA, 143 bp, Artificial sequence
  Synthetic
ggcccagactgagcacgtgagttttagagctagaaatagcaagttaaaataaggctagtccgttatcaacttgaaaaagtggcaccgagtcggtgcatatgaccacccacctaattaaaggagggcaagtcgtgctcagtctg

SEQ ID NO: 28 - DNA, 137 bp, Artificial sequence
  Synthetic
attaaaggagggcaagtgctgttttagagctagaaatagcaagttaaaataaggctagtccgttatcaacttgaaaaagtggcaccgagtcggtgctcaatccttggggcccagactgagcacgacttgccctcctt

SEQ ID NO: 29 - DNA, 141 bp, Artificial sequence
  Synthetic
gcattttcaggaggaagcgagttttagagctagaaatagcaagttaaaataaggctagtccgttatcaacttgaaaaagtggcaccgagtcggtgcagttaaggataactcagacacaggcattccggcttcctcctgaaa

SEQ ID NO: 30 - DNA, 138 bp, Artificial sequence
  Synthetic
agacacaggcattccgggcagttttagagctagaaatagcaagttaaaataaggctagtccgttatcaacttgaaaaagtggcaccgagtcggtgcttcagaagagggtgcattttcaggaggaagccggaatgcctg

SEQ ID NO: 31 - DNA, 137 bp, Artificial sequence
  Synthetic
gagtccgagcagaagaagaagttttagagctagaaatagcaagttaaaataaggctagtccgttatcaacttgaaaaagtggcaccgagtcggtgctttattattcccatagggaagggggacattcttctgctcgg

SEQ ID NO: 32 - DNA, 136 bp, Artificial sequence
  Synthetic
catagggaagggggacactggttttagagctagaaatagcaagttaaaataaggctagtccgttatcaacttgaaaaagtggcaccgagtcggtgcaggaagggcctgagtccgagcagaagaatgtcccccttcc

SEQ ID NO: 33 - DNA, 141 bp, Artificial sequence
  Synthetic
aagcatgatcagaacggttggttttagagctagaaatagcaagttaaaataaggctagtccgttatcaacttgaaaaagtggcaccgagtcggtgcacacgcagtcctcttttcccagggctcccccgcctaccagtttgc

SEQ ID NO: 34 - DNA, 139 bp, Artificial sequence
  Synthetic
ttcccagggctcccccgagggttttagagctagaaatagcaagttaaaataaggctagtccgttatcaacttgaaaaagtggcaccgagtcggtgcacggctacctagtgagcctgcaaactggtaggcgggggagccc

SEQ ID NO: 35 - DNA, 140 bp, Artificial sequence
  Synthetic
aaggggcatgaagtttactggttttagagctagaaatagcaagttaaaataaggctagtccgttatcaacttgaaaaagtggcaccgagtcggtgcaggtcagagtcctggctctgtgactcagtgataaacttcatgcc

SEQ ID NO: 36 - DNA, 140 bp, Artificial sequence
  Synthetic
gctctgtgactcagtgacctggaatagaaaacaaaagtttaagttattctaaggccagtccggaatcatcctaaaaaggaggcaccgagtcggtgcacatggtacccatgaaggggcatgaagtttatcactgagtcaca

SEQ ID NO: 37 - DNA, 141 bp, Artificial sequence
  Synthetic
aagcatgatcagaacggttggttttagagctagaaatagcaagttaaaataaggctagtccgttatcaacttgaaaaagtggcaccgagtcggtgcacggctacctagtgagcctgcaaactggtaggccgttctgatcat

SEQ ID NO: 38 - DNA, 141 bp, Artificial sequence
  Synthetic
gcctgcaaactggtaggcgcgttttagagctagaaatagcaagttaaaataaggctagtccgttatcaacttgaaaaagtggcaccgagtcggtgctttgactattttagcaagcatgatcagaacggcctaccagtttgc

SEQ ID NO: 39 - DNA, 141 bp, Artificial sequence
  Synthetic
aggttggcccgtaatacctggttttagagctagaaatagcaagttaaaataaggctagtccgttatcaacttgaaaaagtggcaccgagtcggtgcacggctacctagtgagcctgcaaactggtagggtattacgggcca

SEQ ID NO: 40 - DNA, 141 bp, Artificial sequence
  Synthetic
gcctgcaaactggtaggcgcgttttagagctagaaatagcaagttaaaataaggctagtccgttatcaacttgaaaaagtggcaccgagtcggtgcacttcatgtattgtcaggttggcccgtaataccctaccagtttgc

SEQ ID NO: 41 - DNA, 126 bp, Artificial sequence
  Synthetic
aacctctcggctttcccgcggttttagagctagaaatagcaagttaaaataaggctagtccgttatcaacttgaaaaagtggcaccgagtcggtgcagctgctcaccacgacggggaaagccgaga

SEQ ID NO: 42 - DNA, 134 bp, Artificial sequence
  Synthetic
agctgctcaccacgacgccagttttagagctagaaatagcaagttaaaataaggctagtccgttatcaacttgaaaaagtggcaccgagtcggtgcctcaggcgaacctctcggctttccccgtcgtggtgagc

SEQ ID NO: 43 - DNA, 151 bp, Artificial sequence
  Synthetic
aacctctcggctttcccgcggttttagagctagaaatagcaagttaaaataaggctagtccgttatcaacttgaaaaagtggcaccgagtcggtgctgaaccggccagggccggcaggccgagctgctcaccacgacggggaaagccgaga

SEQ ID NO: 44 - DNA, 155 bp, Artificial sequence
  Synthetic
agctgctcaccacgacgccagttttagagctagaaatagcaagttaaaataaggctagtccgttatcaacttgaaaaagtggcaccgagtcggtgcttcaggcggctgcgacgagccctcaggcgaacctctcggctttccccgtcgtggtgagc

SEQ ID NO: 45 - DNA, 126 bp, Artificial sequence
  Synthetic
gcctgcaaactggtaggcgcgttttagagctagaaatagcaagttaaaataaggctagtccgttatcaacttgaaaaagtggcaccgagtcggtgcagctgctcaccacgacgcctaccagtttgc

SEQ ID NO: 46 - DNA, 138 bp, Artificial sequence
  Synthetic
agctgctcaccacgacgccagttttagagctagaaatagcaagttaaaataaggctagtccgttatcaacttgaaaaagtggcaccgagtcggtgcgctacctagtgagcctgcaaactggtaggcgtcgtggtgagc

SEQ ID NO: 47 - DNA, 151 bp, Artificial sequence
  Synthetic
gcctgcaaactggtaggcgcgttttagagctagaaatagcaagttaaaataaggctagtccgttatcaacttgaaaaagtggcaccgagtcggtgctgaaccggccagggccggcaggccgagctgctcaccacgacgcctaccagtttgc

SEQ ID NO: 48 - DNA, 148 bp, Artificial sequence
  Synthetic
agctgctcaccacgacgccagttttagagctagaaatagcaagttaaaataaggctagtccgttatcaacttgaaaaagtggcaccgagtcggtgcaattcccacggctacctagtgagcctgcaaactggtaggcgtcgtggtgagc

SEQ ID NO: 49 - DNA, 139 bp, Artificial sequence
  Synthetic
aacctctcggctttcccgcggttttagagctagaaatagcaagttaaaataaggctagtccgttatcaacttgaaaaagtggcaccgagtcggtgcacgtaaacggccacaagttcagcgtgtccggggaaagccgaga

SEQ ID NO: 50 - DNA, 139 bp, Artificial sequence
  Synthetic
agctgctcaccacgacgccagttttagagctagaaatagcaagttaaaataaggctagtccgttatcaacttgaaaaagtggcaccgagtcggtgcactccagcaggaccatgtgatcgcgcttctcgtcgtggtgagc

SEQ ID NO: 51 - DNA, 139 bp, Artificial sequence
  Synthetic
gcctgcaaactggtaggcgcgttttagagctagaaatagcaagttaaaataaggctagtccgttatcaacttgaaaaagtggcaccgagtcggtgcacgtaaacggccacaagttcagcgtgtccgcctaccagtttgc

SEQ ID NO: 52 - DNA, 139 bp, Artificial sequence; identical to SEQ ID NO: 50
  Synthetic

SEQ ID NO: 53 - DNA, 42 bp, Artificial sequence
  Synthetic
gcgtcagatgtgtataagagacagatggtgagcaagggcgag

SEQ ID NO: 54 - DNA, 41 bp, Artificial sequence
  Synthetic
ttcagacgtgtgctcttccgatctaagatggtgcgctcctg

SEQ ID NO: 55 - DNA, 46 bp, Artificial sequence
  Synthetic
ttcagacgtgtgctcttccgatctacttgtacagctcgtccatgcc

SEQ ID NO: 56 - DNA, 57 bp, Artificial sequence
  Synthetic
  misc_feature (25)..(39): n is a, c, g, or t
gcgtcagatgtgtataagagacagnnnnnnnnnnnnnnnatggtgagcaagggcgag

SEQ ID NO: 57 - DNA, 59 bp, Artificial sequence
  Synthetic
  misc_feature (25)..(39): n is a, c, g, or t
gcgtcagatgtgtataagagacagnnnnnnnnnnnnnnngcctgcttctcctcagcttc

SEQ ID NO: 58 - DNA, 45 bp, Artificial sequence
  Synthetic
ttcagacgtgtgctcttccgatctcattcccgaatctgccctcgg

SEQ ID NO: 59 - DNA, 60 bp, Artificial sequence
  Synthetic
  misc_feature (25)..(39): n is a, c, g, or t
gcgtcagatgtgtataagagacagnnnnnnnnnnnnnnnagcctcggcttcttctgggag

SEQ ID NO: 60 - DNA, 45 bp, Artificial sequence; identical to SEQ ID NO: 58
  Synthetic

SEQ ID NO: 61 - DNA, 46 bp, Artificial sequence
  Synthetic
gcgtcagatgtgtataagagacagcgctcagctccgtttcggtttc

SEQ ID NO: 62 - DNA, 46 bp, Artificial sequence
  Synthetic
ttcagacgtgtgctcttccgatctataagccatcgccgtcacttag

SEQ ID NO: 63 - DNA, 61 bp, Artificial sequence
  Synthetic
  misc_feature (25)..(39): n is a, c, g, or t
gcgtcagatgtgtataagagacagnnnnnnnnnnnnnnntccaaggtgaaagcggaagtag

SEQ ID NO: 64 - DNA, 44 bp, Artificial sequence
  Synthetic
ctgaaggtgatagcggtggcagatcggaagagcacacgtctgaa

SEQ ID NO: 65 - DNA, 69 bp, Artificial sequence
  Synthetic
  misc_feature (25)..(39): n is a, c, g, or t
gcgtcagatgtgtataagagacagnnnnnnnnnnnnnnngcaagtaagcatgcatttgtaggcttgatg

SEQ ID NO: 66 - DNA, 48 bp, Artificial sequence
  Synthetic
ttcagacgtgtgctcttccgatctgggttttccagctgttaagcacag

SEQ ID NO: 67 - DNA, 67 bp, Artificial sequence
  Synthetic
  misc_feature (25)..(39): n is a, c, g, or t
gcgtcagatgtgtataagagacagnnnnnnnnnnnnnnncgctccgaaggtaaaagaaatcattgag

SEQ ID NO: 68 - DNA, 52 bp, Artificial sequence
  Synthetic
ttcagacgtgtgctcttccgatcttctcctgtactctctgccttatagagac

SEQ ID NO: 69 - DNA, 64 bp, Artificial sequence
  Synthetic
  misc_feature (25)..(39): n is a, c, g, or t
gcgtcagatgtgtataagagacagnnnnnnnnnnnnnnngttccagaaccggaggacaaagtac

SEQ ID NO: 70 - DNA, 48 bp, Artificial sequence
  Synthetic
ttcagacgtgtgctcttccgatcttgctgtggagctggaggtagagac

SEQ ID NO: 71 - DNA, 60 bp, Artificial sequence; identical to SEQ ID NO: 59
  Synthetic
  misc_feature (25)..(39): n is a, c, g, or t

SEQ ID NO: 72 - DNA, 45 bp, Artificial sequence; identical to SEQ ID NO: 58
  Synthetic

SEQ ID NO: 73 - DNA, 61 bp, Artificial sequence
  Synthetic
  misc_feature (25)..(39): n is a, c, g, or t
gcgtcagatgtgtataagagacagnnnnnnnnnnnnnnnttgggttggtaactggatgttg

SEQ ID NO: 74 - DNA, 46 bp, Artificial sequence
  Synthetic
ttcagacgtgtgctcttccgatctgggttttcatgtcctctgcttc

SEQ ID NO: 75 - DNA, 60 bp, Artificial sequence; identical to SEQ ID NO: 59
  Synthetic
  misc_feature (25)..(39): n is a, c, g, or t

SEQ ID NO: 76 - DNA, 51 bp, Artificial sequence
  Synthetic
ttcagacgtgtgctcttccgatctctcttacaagccaagtactgtgctaag

SEQ ID NO: 77 - DNA, 60 bp, Artificial sequence; identical to SEQ ID NO: 59
  Synthetic
  misc_feature (25)..(39): n is a, c, g, or t

SEQ ID NO: 78 - DNA, 49 bp, Artificial sequence
  Synthetic
ttcagacgtgtgctcttccgatctgagcatctccttttacaacctaagc

SEQ ID NO: 79 - DNA, 21 bp, Artificial sequence
  Synthetic
cctgcttctcctcagcttcag

SEQ ID NO: 80 - DNA, 23 bp, Artificial sequence
  Synthetic
ttctcttcccacacgcagtcctc

SEQ ID NO: 81 - DNA, 26 bp, Artificial sequence
  Synthetic
tctcggctttccccgtcgtggtgagc

SEQ ID NO: 82 - DNA, 22 bp, Artificial sequence
  Synthetic
ttcccacggctacctagtgagc

SEQ ID NO: 83 - DNA, 23 bp, Artificial sequence
  Synthetic
ttctcttcccacacgcagtcctc

SEQ ID NO: 84 - DNA, 27 bp, Artificial sequence
  Synthetic
tgctcaccacgacgcctaccagtttgc

SEQ ID NO: 85 - DNA, 22 bp, Artificial sequence
  Synthetic
ttcccacggctacctagtgagc

SEQ ID NO: 86 - DNA, 22 bp, Artificial sequence
  Synthetic
gagttacggcggtgattcctgc

SEQ ID NO: 87 - DNA, 28 bp, Artificial sequence
  Synthetic
ctggtaggccgttctgatcatgcttgct

SEQ ID NO: 88 - DNA, 22 bp, Artificial sequence
  Synthetic
ttcccacggctacctagtgagc

SEQ ID NO: 89 - DNA, 23 bp, Artificial sequence
  Synthetic
tgctgtctttcagtccccaaagc

SEQ ID NO: 90 - DNA, 28 bp, Artificial sequence
  Synthetic
ctggtagggtattacgggccaacctgac

SEQ ID NO: 91 - DNA, 69 bp, Homo sapiens
ggcggctgcgacgagccctcaggcgaacctctcggctttcccgcgcggcgccgcctcttgctgcgcctc

SEQ ID NO: 92 - DNA, 65 bp, Homo sapiens
gttatggcgacccgcagccctggcgtcgtggtgagcagctcggcctgccggccctggccggttca

SEQ ID NO: 93 - DNA, 42 bp, Homo sapiens
ggcggctgcgacgagccctcaggcgaacctctcggctttccc

SEQ ID NO: 94 - DNA, 42 bp, Homo sapiens
cgtcgtggtgagcagctcggcctgccggccctggccggttca

SEQ ID NO: 95 - DNA, 139 bp, Artificial sequence
  Synthetic
ggcggctgcgacgagccctcaggcgaacctctcggctttccccgtcgtggtgagcagctcggcctgccggccctcaggcgaacctctcggctttccccgtcgtggtgagcagctcggcctgccggccctggccggttca

SEQ ID NO: 96 - DNA, 67 bp, Homo sapiens
gaaaattcccacggctacctagtgagcctgcaaactggtagggcgcggcgccgcctcttgctgcgcc

SEQ ID NO: 97 - DNA, 68 bp, Homo sapiens
tccgttatggcgacccgcagccctggcgtcgtggtgagcagctcggcctgccggccctggccggttca

SEQ ID NO: 98 - DNA, 42 bp, Homo sapiens
gaaaattcccacggctacctagtgagcctgcaaactggtagg

SEQ ID NO: 99 - DNA, 42 bp, Homo sapiens; identical to SEQ ID NO: 94

SEQ ID NO: 100 - DNA, 140 bp, Artificial sequence
  Synthetic
gaaaattcccacggctacctagtgagcctgcaaactggtaggcgtcgtggtgagcagctcggcctgccggctacctagtgagcctgcaaactggtaggcgtcgtggtgagcagctcggcctgccggccctggccggttca